(19) United States
(12) Patent Application Publication
(10) Pub. No.: US 2023/0357353 A1
(43) Pub. Date: Nov. 9, 2023

(54) MAdCAM-1 POLYPEPTIDES AND USES THEREOF

(71) Applicant: Ramot at Tel-Aviv University Ltd., Tel-Aviv (IL)

(72) Inventors: Niels DAMMES, Tel Aviv (IL); Dan PEER, Tel Aviv (IL)

(73) Assignee: Ramot at Tel-Aviv University Ltd., Tel-Aviv (IL)

(21) Appl. No.: 17/988,893

(22) Filed: Nov. 17, 2022

Related U.S. Application Data
(63) Continuation of application No. PCT/IL2021/050576, filed on May 19, 2021.
(60) Provisional application No. 63/132,510, filed on Dec. 31, 2020.

(51) Int. Cl. C07K 14/705; A61P 29/00; G01N 33/50

(52) U.S. Cl. CPC C07K 14/705; A61P 29/00; G01N 33/5008; C07K 2319/30

(57) ABSTRACT

MAdCAM-1 polypeptides are provided. Accordingly there is provided a polypeptide comprising an amino acid sequence of MAdCAM-1 capable of binding alpha4beta7 integrin, wherein said polypeptide is in association with a therapeutic or a detectable moiety. Also provided are methods of treating and diagnosing inflammation mediated by MAdCAM-1.

### RELATED APPLICATIONS

This application is a Continuation of PCT Patent Application No. PCT/IL2021/050576 having International filing date of May 19, 2021, which claims the benefit of priority under 35 USC § 119(e) of U.S. Provisional Patent Application Nos. 63/026,793 filed on May 19, 2020 and 63/132,510 filed on Dec. 31, 2020. The contents of the above applications are all incorporated by reference as if fully set forth herein in their entirety.

The work leading to this invention has received ERC Consolidator Grant funding: Consolidator Grant (CoG), LS7, under grant agreement No. ERC-2014-CoG.

### SEQUENCE LISTING STATEMENT

The XML file, entitled 94381SecondReplacementSequenceListing.xml, created on Jul. 10, 2023, comprising 82,682 bytes, submitted concurrently with the filing of this application is incorporated herein by reference.

### FIELD AND BACKGROUND OF THE INVENTION

The present invention, in some embodiments thereof, relates to MAdCAM-1 polypeptides and uses thereof.

Mucosal Vascular Addressin Cell Adhesion Molecule 1 (MAdCAM-1) is a cell-surface immunoglobulin (Ig) superfamily member composed of two extracellular Ig-like domains, followed by a mucin-like domain, a transmembrane domain, and a short cytoplasmic domain. The murine MAdCAM-1 comprises an additional Ig-like domain. MAdCAM-1 specifically binds the lymphocyte homing receptor α4β7 integrin.

The α4β7 integrin has several ligands, including MAdCAM-1, fibronectin and vascular cell adhesion molecule-1 (VCAM-1). Like other integrins, the functionality of α₄β₇ depends on its conformational state¹². That is, integrins change conformation upon specific stimuli (e.g. chemokines) which induces a specific “inside-out signaling” that changes the integrin's inactive conformation (a low-affinity bent conformation) into its active form, characterized by a high-affinity (HA) extended conformation which may be distinct for different ligands¹³. Thus, whether integrin α₄β₇ has HA for MAdCAM-1 or e.g. VCAM-1 depends on the stimulus received; and while a large percentage of circulating lymphocytes express α₄β₇, only a fraction of these cells utilize a conformation enabling binding to MAdCAM-1.

MAdCAM-1 has been shown to be expressed at sites of lymphocyte extravasation. In particular, MAdCAM-1 expression was reported in vascular endothelial cells of mucosal tissues, including gut-associated tissues or lymphoid organs (e.g. Peyer's patches, Mesenteric lymph nodes and venules of the lamina propria of the small and large intestine) and the lactating mammary gland. MAdCAM-1 expression is upregulated on endothelia, especially high endothelial venules (HEV), in a variety of chronic inflammatory diseases, and may mediate increased leukocyte trafficking into inflamed tissues. Consequently, MAdCAM-1 binding to α4β7 was shown to mediate the progression of e.g. inflammatory bowel disease (IBD), Type I diabetes, chronic inflammatory liver disease and a chronic progressive form of EAE. IBD (such as ulcerative colitis and Crohn's disease), for example, is a chronic disease involving inflammation of the gastrointestinal tract. IBD is a growing problem with rising incidence since the 19^{th} century, affecting an estimated two million people in the United States alone. Symptoms include abdominal pain, cramping, diarrhea and rectal bleeding. Current IBD treatments includes anti-inflammatory drugs (such as, corticosteroids and sulfasalazine), immunosuppressive drugs (such as, 6-mercaptopurine, cyclosporine and azathioprine), anti-TNFα and surgery (such as, colectomy).

Antibodies against α4β7 integrin or MAdCAM-1 and inhibitory polypeptides that interfere with α4β7 integrin binding to MAdCAM-1 in conformational sensitive or insensitive manner have been suggested in the art (see e.g. U.S. Pat. Nos. 7,147,851, 6,037,324, 8,277,808, US patent application Publication No. US20070202097; Yang et al. Scand J Immunol. 1995; 42: 235-47 and Qi et al. J Biol Chem. 2012 May 4; 287(19):15749-59).

Additional background art includes
Peer, D. et al. Science 319, 627-30 (2008);
Dearling, J. L. J. et al. Inflamm. Bowel Dis. 16, 1458-1466 (2010); and
Dearling, J. L. J. et al. Inflamm. Bowel Dis. 22, 529-538 (2016).

### SUMMARY OF THE INVENTION

According to an aspect of some embodiments of the present invention there is provided a polypeptide comprising an amino acid sequence of MAdCAM-1 capable of binding α4β7 integrin, wherein the polypeptide is in association with a therapeutic moiety, wherein the therapeutic moiety is not a constant region of an antibody.

According to an aspect of some embodiments of the present invention there is provided a targeted particle comprising a therapeutic and/or a detectable moiety, the particle is attached to a polypeptide comprising an amino acid sequence of MAdCAM-1.

According to some embodiments of the invention, the amino acid sequence of MAdCAM-1 does not comprise a functional Mucin-like domain.

According to some embodiments of the invention, the amino acid sequence of MAdCAM-1 is more than 5 amino acids in length.

According to some embodiments of the invention, the therapeutic moiety is attached to the polypeptide via a linker.

According to some embodiments of the invention, the therapeutic moiety is attached to-, or encapsulated in a particle.

According to some embodiments of the invention, the particle is a lipid particle.

According to some embodiments of the invention, the therapeutic moiety is a polynucleotide, a small molecule or a polypeptide.

According to some embodiments of the invention, the therapeutic moiety is an RNA silencing agent.

According to some embodiments of the invention, the therapeutic moiety downregulates expression and/or activity of a pro-inflammatory cytokine.

According to an aspect of some embodiments of the present invention there is provided a method of treating a disease associated with inflammation mediated by MAdCAM-1 in a subject in need thereof, the method comprising administering to the subject a therapeutically effective amount of the polypeptide or the particle, thereby treating the disease associated with inflammation mediated by MAdCAM-1.

According to an aspect of some embodiments of the present invention there is provided the polypeptide or the particle, for use in treating a disease associated with inflammation mediated by MAdCAM-1.

According to some embodiments of the invention, the disease is inflammatory bowel disease (IBD).

According to an aspect of some embodiments of the present invention there is provided a polypeptide comprising an amino acid sequence of MAdCAM-1 capable of binding α4β7 integrin, wherein the polypeptide is in association with a detectable moiety, wherein the amino acid sequence of MAdCAM-1 is more than 5 amino acids long and does not comprise a functional Mucin-like domain.

According to some embodiments of the invention, the detectable moiety is attached to the polypeptide via a linker.

According to some embodiments of the invention, the MAdCAM-1 is human MAdCAM-1.

According to some embodiments of the invention, the amino acid sequence of MAdCAM-1 comprises an amino acid sequence of a first Ig-like domain (D1) located N to C in a full length MAdCAM-1.

According to some embodiments of the invention, the amino acid sequence of MAdCAM-1 comprises an amino acid sequence of a second Ig-like domain (D2) located N to C in a full length MAdCAM-1.

According to some embodiments of the invention, the amino acid sequence of MAdCAM-1 comprises an amino acid sequence of a first Ig-like domain (D1) and a second Ig-like domain (D2) located N to C in a full length MAdCAM-1.

According to some embodiments of the invention, the amino acid sequence of MAdCAM-1 comprises (SEQ ID NO: 21).

According to some embodiments of the invention, the amino acid sequence of MAdCAM-1 comprises SEQ ID NO: 22.

According to an aspect of some embodiments of the present invention there is provided a method of diagnosing inflammation mediated by MAdCAM-1 in a subject in need, the method comprising:
(a) administering to the subject the polypeptide or the particle; and
(b) determining an amount of the polypeptide or the particle in a suspected inflamed tissue of the subject following the administering,

wherein an amount above a predetermined threshold is indicative of presence of inflammation mediated by MAdCAM-1 in the tissue of the subject.

According to an aspect of some embodiments of the present invention there is provided a method of monitoring efficacy of treatment against inflammation mediated by MAdCAM-1 in a subject in need, the method comprising:
(a) administering to the subject the polypeptide or the particle following the treatment; and
(b) determining an amount of the polypeptide or the particle in a suspected inflamed tissue of the subject following the administering,

wherein a decrease in the amount following the treatment beyond a predetermined threshold is indicative of reduction in inflammation and efficaciousness of the treatment.

According to some embodiments of the invention, the method comprising treating the subject with the treatment prior to the (a).

According to some embodiments of the invention, the tissue is a gut tissue.

According to some embodiments of the invention, the inflammation is associated with inflammatory bowel disease (IBD).

According to some embodiments of the invention, the polypeptide comprises an amino acid sequence of a constant region of an antibody.

According to some embodiments of the invention, the constant region of an antibody is an Fc domain or a fragment thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E demonstrate generation of MAdCAM-1-Fc and lipid nanoparticles (LNPs) that carry siRNA payloads having the MAdCAM-1-Fc as a targeting moiety. FIG. 1A is a schematic representation of generation of LNPs using microfluidics. The ionizable lipid facilitates siRNA encapsulation through its positive charge at low pH. FIG. 1B is a schematic representation of the different domains of the MAdCAM-1-Fc fusion protein. FIG. 1C is a schematic representation of the fusion strategy. The different domains of the native MAdCAM-1 are indicated. Briefly, for generating the fusion protein, MAdCAM-1 integrin binding domains D1 and D2 (D1D2) are fused to the hinge of rat IgG₂ₐ with a flexible linker. FIG. 1D is a schematic representation of conjugation of the MAdCAM-1-Fc to the LNPs. Briefly, a monoclonal antibody against rat IgG₂ₐ (RG7 linker) is chemically conjugated to the maleimide group in the lipid DSPE-PEG-Maleimide in the LNPs. RG7 readily binds the MAdCAM-1-Fc by antibody affinity. FIG. 1E is a schematic representation of LNP targeting to HA α₄β₇ integrin, following induction of a conformational change by e.g. CCL25.

FIGS. 2A-B demonstrate characterization of the generated MAdCAM-1-Fc fusion proteins. FIG. 2A shows SDS-PAGE analysis of D1D2-Fc (WT) and mD1D2-Fc (MUT) proteins. Dimerization through disulfide bonds is evident when comparing to the lanes without reducing agent. FIG. 2B demonstrates in-vitro binding of the purified D1D2-Fc (D1D2) and D1D2-Fc (mD1D2) proteins to TK-1 cells in the presence or absence of pre-treatment with Mn²⁺, as determined by flow cytometry. Quantification of geometric mean of fluorescence demonstrates a significant difference between D1D2 and mD1D2 in Mn²⁺-activated cells (p=0.0001, n=4, Student's t-test).

FIGS. 2C-F demonstrate characterization of the generated lipid nanoparticles (LNPs). FIG. 2C shows representative TEM images of unconjugated LNPs and LNPs conjugated to RG7. FIG. 2D shows siRNA entrapment efficiency assayed by Ribogreen. FIG. 2E demonstrates in-vitro binding of Cy5-labeled D1D2-targeted LNPs to TK1 cells as compared to Cy5-labeled mD1D2-targeted LNPs. FIG. 2F shows representative confocal images showing binding and internalization of Cy5-labeled D1D2-targeted LNPs into TK-1 cells.

FIGS. 3A-D demonstrate molecular imaging of inflammatory leukocytes in experimental colitis using PET/CT and D1D2-NOTA-^{Γ}Cu. FIG. 3A shows graphs demonstrating that conjugation of NOTA did not affect functionality of the D1D2-Fc protein, as determined by binding to TK-1 cells. mD1D2 control-mD1D2-Fc, Free D1D2-D1D2-Fc, NOTA-D1D2-NOTA conjugated D1D2-Fc. FIG. 3B shows representative images of a series of molecular PET/CT imaging studies using ⁶⁴Cu radiolabeled NOTA conjugated D1D2-Fc (NOTA-D1D2) injected into healthy or colitic mice, as compared to injection of ⁶⁴Cu radiolabeled NOTA conjugated mD1D2-Fc (NOTA-mD1D2). Both coronal and transaxial images are displayed. Enhanced gut uptake by the D1D2-Fc in mice with active colitis is demonstrated. FIG. 3C demonstrates that treatment with NOTA-conjugated D1D2-Fc or mD1D2-Fc did not significantly affect colitis severity as determined by colon density and analyzed by one-way ANOVA. There was a significant difference (p<0.001, n=10) in colon density between piroxicam-treated animals and controls, confirming active colitis. FIG. 3D demonstrates that uptake of radiolabeled D1D2-Fc increases with degree of colitic disease in a piroxicam-induced mouse model of colitis. Protein uptake is plotted against colonic density (degree of colitis) for each of the 3 tissue types of major interest (large intestine in blue, small intestine in red, stomach in green). For the animals with colitis, uptake of the target-binding protein increased with colitis severity (solid lines to right of the graph), while for the control animals, protein uptake decreased (dashed lines to left). The slopes (se) for the large intestine, small intestine, and stomach were positive for colitis mice (0.88 (0.57), 1.61 (0.71), and 0.36 (0.59)), and negative for control mice (−7.39 (4.94), −6.60 (6.12), and −8.32 (5.07)). The (colitis-control) differences (se) of these slopes were similar across the three tissues: 8.27 (4.98), 8.20 (6.16), and 8.68 (5.11) for the large intestine, small intestine, and stomach, respectively. The similarity of (colitis-control) differences of slopes was confirmed by fitting a 2-way interaction mixed model (containing fixed and random effects) using the data for all tissue types. The common estimate (se) based on this model was 8.38 (3.41). The p-value corresponding to the null hypothesis of equal colitis and control slopes was p=0.014, indicating >95% confidence that there is a difference between the slopes of the two populations.

FIGS. 4A-D demonstrate in-vivo gene silencing of CD45 in healthy mice and mice with colitis using D1D2-targeted LNPs encapsulating CD45 siRNA (siCD45, SEQ ID NO: 20, 41), as compared to D1D2-LNPs loaded with negative control siRNA (siNC, SEQ ID NO: 18-19), mD1D2-LNPs loaded with siCD45 (SEQ ID NO: 20, 41) and mock control. FIG. 4A shows gene silencing in the CD4⁺ T-cell population from the mLN in the piroxicam-accelerated colitis (PAC) model. Observed gene silencing was ˜15% D1D2-targeted LNPs encapsulating siCD45 as compared to control mD1D2-targeted LNPs encapsulating siCD45. FIG. 4B demonstrates no significant gene silencing in healthy mice in any cell population. FIG. 4C demonstrates significant CD45 gene silencing in CD4⁺ and CD19⁺ cells of PAC mice. Data are the mean with error bars displaying the standard deviation, n=5 mice per group, * p<0.05, n.s.=not significant (statistics performed with Student's t-test comparing D1D2 to mD1D2 for each cell subtype). FIG. 4D demonstrates that silencing levels of colonic CD4⁺ T-cells were not significant.

FIG. 5A-D demonstrate safety profile of three LNP formulations: D1D2-LNPs loaded with siNC (SEQ ID NO: 18-19); D1D2-LNPs loaded with siCD45 (SEQ ID NO: 20, 41) and mD1D2-LNPs loaded with siCD45 (SEQ ID NO: 20, 41) as compared to mock control. Safety was determined by measuring both liver toxicity and immune activation in healthy mice following injection of the LNPs. FIG. 5A shows expression levels of the pro-inflammatory cytokines TNF-α and IL-6 in spleens of treated mice, as determined by ELISA. FIG. 5B shows serum levels of the liver enzymes AST, ALT and ALP. FIG. 5C shows whole blood count. FIG. 5D shows representative histology of the liver. The liver was sectioned and stained with H&E. No significant elevation in cytokines expression, liver enzymes and blood count could be detected (one-way ANOVA, with Dunnett's test comparing each treatment to the untreated mice, n=5). There was only a significant decrease in ALP levels compared to the untreated group. Liver histology did not reveal tissue damage or excessive bleeding in any of the treated groups.

FIGS. 6A-H demonstrate in-vivo therapeutic gene silencing of IFNγ using D1D2-targeted LNPs in the PAC mouse model. Treatment groups included healthy control, mock control, D1D2-targeted LNPs loaded with siIFNγ (SEQ ID NO: 9-10), mD1D2-targeted LNPs loaded with siIFNγ (SEQ ID NO: 9-10), D1D2-LNPs loaded with a control siNC (SEQ ID NO: 18-19) and anti-TNF-α control. FIG. 6A is a schematic representation of the experimental design. FIG. 6B shows percentage of weight change in all treated groups. Error bars represent the standard error of the mean. In two groups, the error bars are smaller than the symbols and are therefore not displayed. FIGS. 6C-F demonstrate expression levels of the indicated pro-inflammatory cytokines in the colon (FIGS. 6C-D) and blood (FIGS. 6E-F). FIG. 6G demonstrates colon length. FIG. 6H demonstrates colon histological score index. Data are represented as scatter dot plot with error bars representing the 95% CI, statistical tests were calculated using one-way ANOVA with Dunnet's post hoc test, n=12 mice/group, * p<0.05, ** p<0.01, *** p<0.0001. Statistical significance in the plots indicates a difference in the siIFNγ D1D2-targeted LNP group compared to the negative control groups (siIFNγ-mD1D2, siNC-D1D2 and untreated mice with colitis). Additionally, FIG. 6C indicates a significant difference specifically between the siIFNγ-D1D2 and the mAb-TNF-α groups.

FIG. 7 demonstrates ex vivo binding of D1D2-targeted LNPs or DATK32-targeted LNPs to leukocytes from the spleen and mesenteric lymph nodes (mLN) of healthy mice. Binding was analyzed by flow cytometry and compared to the negative control (isotype control for DATK32, mD1D2 for D1D2), which is displayed in grey. Binding to Mn²⁺-treated cells was compared to cells without Mn²⁺ treatment to verify conformational specificity.

FIG. 8 demonstrates ex vivo binding of D1D2-targeted LNPs or DATK32-targeted LNPs to leukocytes from the spleen and mesenteric lymph nodes (mLN) of mice with colitis. Binding was analyzed by flow cytometry and compared to the negative control (isotype control for DATK32, mD1D2 for D1D2), which is displayed in grey. Binding to Mn²⁺-treated cells was compared to cells without Mn²⁺ treatment to verify conformational specificity.

FIG. 9 shows dot blot analysis confirming the presence of RG7 on the LNP surface. Samples were loaded on a nitrocellulose membrane in triplicates. LNPs encapsulating siCD45 (SEQ ID NO: 20, 41) were compared to LPNs encapsulating siNC (SEQ ID NO: 18-19). Unconjugated LNPs represent the negative control (no RG7). Two different amounts of LNPs were loaded in the wells (0.1 μL and 0.01 μL).

FIG. 10 shows silencing efficiencies of 3 different in silico optimized DsiRNA sequences against mouse IFNγ. Each DsiRNA sequence was transfected into HEK293 cells that stably express murine IFNγ. 48 hours post transfection, cells were lysed and extracted RNA was used for reverse transcription followed by qPCR. DsiRNA sequence #3 (SEQ ID NO: 9-10) was selected as the most optimal sequence.

FIG. 11 shows nucleotide sequence of the open reading frame of the MAdCAM-D1D2-Fc construct. The MAdCAM domains D1 and D2 are underlined. The base that was changed for the mutated control is indicated in bold.

FIG. 12 shows protein sequence of the MAdCAM-D1D2-Fc construct. The MAdCAM domains D1 and D2 are underlined. The amino acid that was changed for the mutated control is indicated in bold.

FIG. 13 demonstrates the flow cytometry gating strategy for cells obtained from the mesenteric lymph nodes. Input gate for each graph is indicated above the graph in square brackets. Gated area of each plot is surrounded by a rectangle and the name of the created gate is indicated within the plot area.

FIG. 14 demonstrates the flow cytometry gating strategy for cells obtained from the spleen. Input gate for each graph is indicated above the graph in square brackets. Gated area of each plot is surrounded by a rectangle and the name of the created gate is indicated within the plot area.

FIG. 15 demonstrates in vitro incubation of D1D2-conjugated LNPs (through the RG7 linker) in mouse blood plasma at 37° C. followed by testing the LNP binding capability to TK-1 cells.

FIGS. 16A-C shows comparison of LNP functionality between different conjugation methods, as determined by binding to TK-1 cells in vitro. FIG. 16A demonstrates direct conjugation of the D1D2 protein by reducing the cysteine residues in the D1D2 protein followed by covalent attachment to the maleimide groups on the DSPE-PEG lipid on the LNP. FIG. 16B demonstrates conjugation using the ASSET linker (lipidated scFv against rat IgG₂ₐ-Fc) which in turn binds the D1D2 protein by affinity to the Fc domain. FIG. 16C demonstrates chemical conjugation of the RG7 monoclonal antibody (mouse anti rat IgG₂ₐ) which in turn binds the D1D2 protein by affinity to the Fc domain.

FIGS. 17A-B demonstrate binding of fluorescently labeled TK-1 cells to a monolayer of HEK293 cells stably expressing recombinant MAdCAM-D1D2 on the membrane. FIG. 17A shows that without activation, TK-1 cells do not adhere to the cell monolayer while FIG. 17B shows that activated cells do adhere.

FIGS. 18A-B show quantification of ex vivo binding of D1D2-targeted LNPs or DATK32-targeted LNPs to primary leukocytes from healthy mice (shown in FIGS. 17A-B). FIG. 18A demonstrates that besides inconsistent results in the CD4⁺ population, the DATK32 binds leukocytes regardless of their integrin conformation. FIG. 18B demonstrates that the D1D2 only binds leukocytes when the integrins are activated. In each experiment, tissues from 3 different mice were used. Data shows mean fluorescence intensity with error bars displaying the 95% CI, n=3, * p<0.05, ** p<0.01, *** p<0.001, n.s.=not significant.

FIGS. 19A-B show quantification of ex vivo binding of D1D2-targeted LNPs or DATK32-targeted LNPs to primary leukocytes from mice with colitis (shown in FIGS. 18A-B). FIG. 19A demonstrates that besides inconsistent results in the CD4⁺ population, the DATK32 binds leukocytes regardless of their integrin conformation. FIG. 19B demonstrates that the D1D2 only binds leukocytes when the integrins are activated. In each experiment, tissues from 3 different mice were used. Data shows mean fluorescence intensity with error bars displaying the 95% CI, n=3, * p<0.05, ** p<0.01, *** p<0.001, n.s.=not significant.

FIG. 20 shows binding of Cy5-labeled LNPs to splenocytes, as determined by flow cytometry.

FIG. 21 demonstrates that chemokine activation using CCL25 specifically increases D1D2-targeted LNPs binding to primary leukocytes when compared to CXCL10-treated control cells.

FIG. 22 demonstrates that the different LNP formulations of the CD45 silencing experiment do not alter the colon visibly in the PAC-induced colitis model, as determined by histology.

FIG. 23 shows representative photomicrographs of colon cross sections for each group of the therapeutic efficacy experiment using siRNA against IFNγ (see detailed description of the groups in the description of FIGS. 6A-H hereinabove).

### DESCRIPTION OF SPECIFIC EMBODIMENTS OF THE INVENTION

The present invention, in some embodiments thereof, relates to MAdCAM-1 polypeptides and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Mucosal Vascular Addressin Cell Adhesion Molecule 1 (MAdCAM-1) is a cell-surface immunoglobulin (Ig) superfamily member which specifically binds the lymphocyte homing receptor α4β7 integrin. The α4β7 integrin has several ligands, including MAdCAM-1, fibronectin and vascular cell adhesion molecule-1 (VCAM-1). Like other integrins, the functionality of α₄β₇ depends on its conformational state. Thus, whether integrin α₄β₇ has high affinity (HA) for MAdCAM-1 or e.g. VCAM-1 depends on the specific stimulus received; and while a large percentage of circulating lymphocytes express αα₄β₇, only a fraction of these cells utilize a conformation enabling binding to MAdCAM-1.

Whilst reducing specific embodiments of the present invention to practice, the present inventors have conceived a novel strategy to targeted delivery of therapeutic or detectable moieties to cells expressing α4β7 integrin in a conformation-sensitive manner, using a polypeptide comprising MAdCAM-1 amino acid sequence as a targeting moiety.

As is illustrated hereinunder and in the examples section, which follows, the present inventors generated a recombinant protein comprising the two Ig-like domains (D1 and D2) of MAdCAM-1 fused to an FC domain of an antibody attached to a lipid nanoparticle (LNP) encapsulating an IFNγ siRNA as a therapeutic moiety or attached to a microPET/CT detectable moiety (⁶⁴Cu) (Examples 1-3 of the Examples section which follows). Using these compositions the present inventors were able to knockdown IFNγ in a selective subset of leukocytes expressing the HA α₄β₇ and induce a therapeutic effect in an inflammatory bowel (IBD) disease mouse model (Example 3 of the Examples section which follows); and to analyze the biodistribution of the composition using microPET/CT (Example 1 of the Examples section which follows).

Consequently, specific embodiments of the present invention suggest compositions comprising MAdCAM-1 polypeptides in association with therapeutic or detectable moieties and their use in treating, diagnosing and monitoring treatment efficacy in conditions associated with inflammation mediated by MAdCAM-1.

Thus, according to an aspect of the present invention, there is provided a polypeptide comprising an amino acid sequence of MAdCAM-1 capable of binding α4β7 integrin, wherein said polypeptide is in association with a therapeutic moiety, wherein said therapeutic moiety is not a constant region of an antibody.

According to an aspect of the present invention, there is provided a polypeptide comprising an amino acid sequence of MAdCAM-1 capable of binding α4β7 integrin, wherein said polypeptide is attached to a therapeutic moiety, wherein said therapeutic moiety is not a constant region of an antibody.

According to an additional or an alternative aspect of the present invention, there is provided a polypeptide comprising an amino acid sequence of MAdCAM-1 capable of binding α4β7 integrin, wherein said polypeptide is in association with a detectable moiety, wherein said amino acid sequence of MAdCAM-1 is more than 5 amino acids long and does not comprise a functional Mucin-like domain.

According to an aspect of the present invention, there is provided a polypeptide comprising an amino acid sequence of MAdCAM-1 capable of binding α4β7 integrin, wherein said polypeptide is attached to a detectable moiety, wherein said amino acid sequence of MAdCAM-1 is more than 5 amino acids long and does not comprise a functional Mucin-like domain.

As used herein, the term “polypeptide” or “peptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

When referring to “an amino acid sequence” the meaning is to the chemical embodiment of the term and not the literal embodiment of the term.

As used herein the term “MAdCAM-1” (also known as addressin and mucosal vascular addressin cell adhesion molecule 1) refers to the polypeptide of the MADCAM1 gene (Gene ID 8174). According to specific embodiments, the MAdCAM-1 is the mouse MAdCAM-1, such as provided in the following Accession Nos. BAA23364, NP_001345714, NP_038619 (SEQ ID NO: 23-25). According to specific embodiments, the MAdCAM-1 is the human MAdCAM-1, such as provided in the following Accession Nos. NP_570116, NP_570118 (SEQ ID NO: 26-27).

As used herein, the phrase “an amino acid sequence of MAdCAM-1” refers to full length MAdCAM-1 or a fragment thereof or a homolog thereof which maintains at least the α4β7 integrin binding capability of the full length MAdCAM-1.

The homolog (naturally occurring or synthetically/recombinantly produced) can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide provided in SEQ ID NO: 23-27 or a functional fragment thereof which exhibit the desired activity (i.e., at least binding a α4β7 integrin); or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

Sequence identity or homology can be determined using any protein or nucleic acid sequence alignment algorithm such as Blast, ClustalW, and MUSCLE.

The homolog may also refer to an ortholog, a deletion, insertion, or substitution variant, including a conservative and non-conservative amino acid substitution, as further described hereinbelow.

According to specific embodiments, the amino acid sequence of MAdCAM-1 may comprise conservative and/or non-conservative amino acid substitutions.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOHF]—CO— for aspartic acid. Those non-conservative substitutions which fall under the scope of the present invention are those which still constitute an amino acid sequence capable of binding α4β7 integrin.

According to specific embodiments, the amino acid sequence of MAdCAM-1 does not comprise a mutation at a D62 residue corresponding to MAdCAM-1 SEQ ID NO: 25.

According to specific embodiments, the amino acid sequence of MAdCAM-1 does not comprise a D62A mutation corresponding to MAdCAM-1 SEQ ID NO: 25.

As used herein, the phrase “corresponding to SEQ ID NO:” intends to include the corresponding amino acid residue in the recited SEQ ID NO relative to any other MAdCAM-1 amino acid sequence.

According to specific embodiments, the amino acid sequence of MAdCAM-1 binds α4β7 integrin with a higher selectivity, as compared to the full length MAdCAM-1.

As used herein, the term “selectivity” refers to the ability to bind α4β7 integrin and not other proteins (e.g. L-selectin, VLA-4), which may be manifested as higher affinity (e.g., K_{d}) to α4β7 integrin as compared to other proteins. Increased affinity can be, for examples, of at least 5, 10, 100, 1000 or 10000 fold.

According to specific embodiment, the amino acid sequence of MAdCAM-1 is a fragment of MAdCAM-1 with reduced binding to L-selectin and/or VLA-4, as compared to the full length MAdCAM-1.

According to specific embodiment, the amino acid sequence of MAdCAM-1 is a fragment of MAdCAM-1 not capable of binding L-selectin, as compared to the full length MAdCAM-1.

According to specific embodiment, the amino acid sequence of MAdCAM-1 is a fragment of MAdCAM-1 not capable of binding VLA-4, as compared to the full length MAdCAM-1.

Assays for testing binding are well known in the art and include, but not limited to flow cytometry, BiaCore, bio-layer interferometry Blitz® assay, HPLC, surface plasmon resonance.

According to specific embodiments, the amino acid sequence of MAdCAM-1 binds α4β7 with a Kd≥10⁻⁵, 10⁻⁴ or 10⁻³.

According to specific embodiments, the amino acid sequence of MAdCAM-1 binds α4β7 with a Kd of 1-100 nM.

As used herein, the term “α4β7 integrin (also known as LPAM-1)” refers to a cell membrane heterodimer protein composed of an α4 chain encoded by the ITGA4 gene (Gene ID 3676) and a β7 chain encoded by the ITGB7 gene (Gene ID 3695). According to specific embodiments, the α4β7 integrin refers to the mouse the α4β7 integrin. According to specific embodiments, the α4β7 integrin refers to the human α4β7 integrin.

Binding of MAdCAM-1 to α4β7 is conformation dependent. α4β7, like other integrins change conformation upon specific stimuli (e.g. chemokines) which induces a specific “inside-out signaling” that changes the integrin's inactive conformation (a low-affinity bent conformation) into its active form, characterized by a high-affinity (HA) extended conformation which may be distinct for different ligands. Hence, according to specific embodiments, testing binding to α4β7 is effected by assessing binding to cells (e.g. T cells e.g. TK-1 cells) following treatment that induces formation of a high affinity conformation e.g. Mn²⁺, CCL25 or PMA.

In this respect, according to specific embodiments, binding is not to a linear domain.

According to specific embodiments, the amino acid sequence of MAdCAM-1 is at least 4 amino acids in length, at least 5 amino acids in length, at least 6 amino acids in length, at least 7 amino acids in length, at least 8 amino acids in length, at least 9 amino acids in length, at least 10 amino acids in length, at least 15 amino acids in length, at least 20 amino acids in length, at least 30 amino acids in length at least 50 amino acids in length at least 80 amino acids in length, at least 100 amino acids in length, at least 150 amino acids in length.

According to a specific embodiment, the amino acid sequence of MAdCAM-1 is more than 5 amino acids in length.

According to specific embodiments, the amino acid sequence of MAdCAM-1 is less than 406 amino acids in length, less than 350 amino acids in length, less than 300 amino acids in length, less than 250 amino acids in length, less than 210 amino acids in length or less than 200 amino acids in length, each possibility represents a separate embodiment of the present invention.

According to a specific embodiment, the amino acid sequence of MAdCAM-1 is 6-199 amino acids in length.

According to a specific embodiment, the amino acid sequence of MAdCAM-1 is 6-195 amino acids in length.

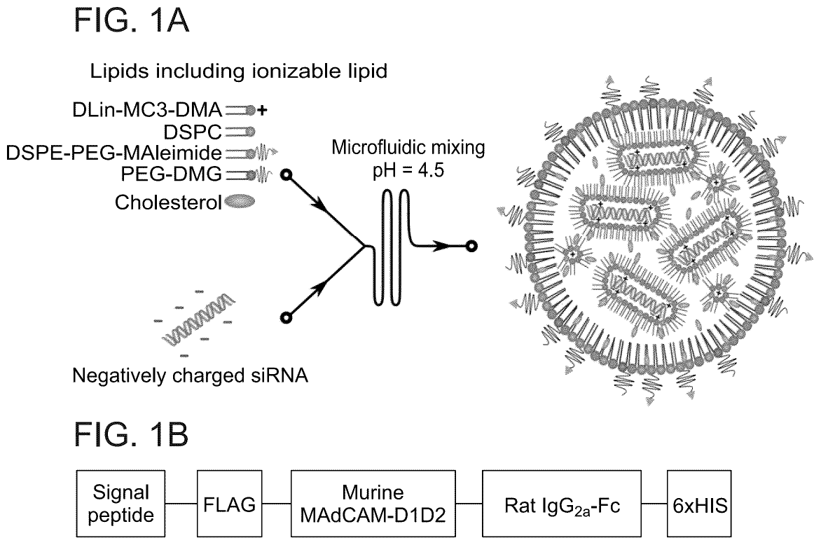

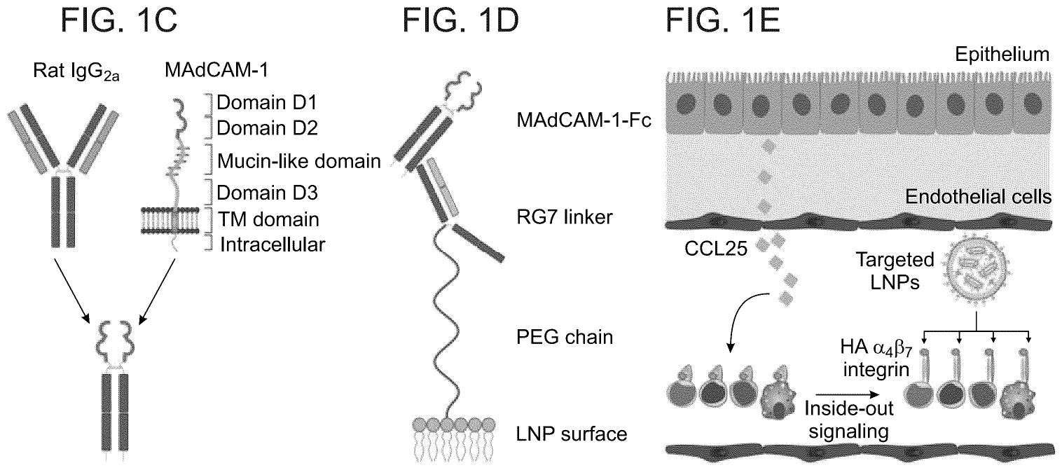

MAdCAM-1 comprises an extracellular domain, a transmembrane domain and an intracellular domain. The extracellular domain of MAdCAM-1 comprises two Ig-like domains (D1 and D2) and Mucin-like domain. In some species, e.g. mouse, MAdCAM-1 also comprises an additional Ig-like domain (D3). A schematic representation is shown in FIG. 1C.

According to specific embodiments, the amino acid sequence of MAdCAM-1 comprises an amino acid sequence of an extracellular domain of MAdCAM-1.

As used herein, the phrase “an amino acid sequence of an extracellular domain of MAdCAM-1” refers to the full length extracellular domain of MAdCAM-1 or a fragment thereof or a homolog thereof.

According to specific embodiments, the extracellular domain of MAdCAM-1 corresponds to amino acids coordinates 22-365 of SEQ ID NO: 25.

According to specific embodiments, the extracellular domain of MAdCAM-1 corresponds to amino acids coordinates 23-319 of SEQ ID NO: 26.

According to specific embodiments, the extracellular domain of MAdCAM-1 has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 28-29.

According to specific embodiments, the amino acid sequence of MAdCAM-1 does not comprise an amino acid sequence of a transmembrane and/or an intracellular domain of MAdCAM-1.

As used herein, the phrase “an amino acid sequence of a transmembrane domain of MAdCAM-1” refers to the full length transmembrane domain of MAdCAM-1 or a fragment thereof or a homolog thereof.

According to specific embodiments, the amino acid sequence of a transmembrane domain of MAdCAM-1 refers to the full length transmembrane domain.

According to specific embodiments, the transmembrane domain of MAdCAM-1 corresponds to amino acids coordinates 366-385 of SEQ ID NO: 25.

According to specific embodiments, the transmembrane domain of MAdCAM-1 corresponds to amino acids coordinates 320 339- of SEQ ID NO: 26.

According to specific embodiments, the transmembrane domain of MAdCAM-1 has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 30-31.

As used herein, the phrase “an amino acid sequence of an intracellular domain of MAdCAM-1” refers to the full length intracellular domain of MAdCAM-1 or a fragment thereof or a homolog thereof.

According to specific embodiments, the amino acid sequence of an intracellular domain of MAdCAM-1 refers to the full length intracellular domain.

According to specific embodiments, the intracellular domain of MAdCAM-1 corresponds to amino acids coordinates 386-405 of SEQ ID NO: 25.

According to specific embodiments, the intracellular domain of MAdCAM-1 corresponds to amino acids coordinates 340-382 of SEQ ID NO: 26.

According to specific embodiments, intracellular domain of MAdCAM-1 has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 32-33.

According to specific embodiments, the amino acid sequence of MAdCAM-1 comprises an amino acid sequence of a first Ig-like domain (D1) located N to C in a full length MAdCAM-1.

As used herein, the phrase “amino acid sequence of a first Ig-like domain (D1)” refers to an amino acid sequence of the full length Ig-like domain of MAdCAM-1 which comprises a motif as set forth in RSCB PDB ENTRY ID: 1 gsm, and is the first Ig-like domain located N to C in a full length MAdCAM-1, or a fragment thereof or a homolog thereof.

According to specific embodiments, the amino acid sequence of the first Ig-like domain (D1) refers to the full length first Ig-like domain.

According to specific embodiments, the amino acid sequence of the first Ig-like domain (D1) is at least 4 amino acids in length, at least 5 amino acids in length, at least 6 amino acids in length, at least 7 amino acids in length, at least 8 amino acids in length, at least 9 amino acids in length, at least 10 amino acids in length, at least 20 amino acids in length, at least 30 amino acids in length, at least 50 amino acids in length, at least 70 amino acids in length, at least 80 amino acids in length or at least 90 amino acids in length, each possibility represents a separate embodiment of the present invention.

According to specific embodiments, the first Ig-like domain (D1) corresponds to amino acids coordinates 22-115 of SEQ ID NO: 25.

According to specific embodiments, the first Ig-like domain (D1) corresponds to amino acids coordinates 23-112 of SEQ ID NO: 26.

According to specific embodiments, the first Ig-like domain (D1) has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 34-35.

According to a specific embodiment, the amino acid sequence of MAdCAM-1 comprises SEQ ID NO: 34.

According to a specific embodiment, the amino acid sequence of MAdCAM-1 comprises SEQ ID NO: 35.

According to a specific embodiment, the amino acid sequence of MAdCAM-1 comprises comprises (SEQ ID NO: 21).

According to specific embodiments, the amino acid sequence of MAdCAM-1 comprises an amino acid sequence of a second Ig-like domain (D2) located N to C in a full length MAdCAM-1.

As used herein, the phrase “amino acid sequence of a second Ig-like domain (D2)” refers to an amino acid sequence of the full length Ig-like domain of MAdCAM-1 which comprises a motif as set forth in RSCB PDB ENTRY ID: 1 gsm, and is the second Ig-like domain located N to C in a full length MAdCAM-1, or a fragment thereof or a homolog thereof.

According to specific embodiments, the amino acid sequence of the second Ig-like domain (D2) refers to the full length second Ig-like domain.

According to specific embodiments, the second Ig-like domain (D2) corresponds to amino acids coordinates 116-220 of SEQ ID NO: 25.

According to specific embodiments, the second Ig-like domain (D2) corresponds to amino acids coordinates 113-220 of SEQ ID NO: 26.

According to specific embodiments, the second Ig-like domain (D2) has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 36-37.

According to a specific embodiment, the amino acid sequence of MAdCAM-1 comprises SEQ ID NO: 36.

According to a specific embodiment, the amino acid sequence of MAdCAM-1 comprises SEQ ID NO: 37.

According to specific embodiments, the amino acid sequence of MAdCAM-1 comprises an amino acid sequence of a first Ig-like domain (D1) and a second Ig-like domain (D2) located N to C in a full length MAdCAM-1.

According to specific embodiments, the amino acid sequence of MAdCAM-1 consists of an amino acid sequence of a first Ig-like domain (D1) and a second Ig-like domain (D2) located N to C in a full length MAdCAM-1.

According to specific embodiments, the amino acid sequence of MAdCAM-1 has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 22.

According to a specific embodiment, the amino acid sequence of MAdCAM-1 comprises SEQ ID NO: 22.

According to a specific embodiment, the amino acid sequence of MAdCAM-1 consists of SEQ ID NO: 22.

According to specific embodiments, the amino acid sequence of MAdCAM-1 does not comprise an amino acid sequence of a third Ig-like domain (D3) of MAdCAM-1.

As used herein, the phrase “amino acid sequence of a third Ig-like domain (D3)” refers to an amino acid sequence of the full length Ig-like domain of MAdCAM-1 which comprises a motif as set forth in RSCB PDB ENTRY ID: 1 gsm, and is the third Ig-like domain located N to C in a full length MAdCAM-1, or a fragment thereof or a homolog thereof.

According to specific embodiments, the amino acid sequence of the third Ig-like domain (D3) refers to the full length third Ig-like domain.

According to specific embodiments, the third Ig-like domain (D3) corresponds to amino acids coordinates 258-365 of SEQ ID NO: 25.

According to specific embodiments, the third Ig-like domain (D3) has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38.

According to specific embodiments, the amino acid sequence of MAdCAM-1 does not comprise an amino acid sequence of a mucin-like domain of MAdCAM-1.

As used herein, the phrase “amino acid sequence of mucin-like domain” refers to an amino acid sequence of the full length mucin-like domain of MAdCAM-1 which comprises a motif as set forth in RSCB PDB ENTRY ID: 1 gsm, or a fragment thereof or a homolog thereof.

According to specific embodiments, the amino acid sequence of the mucin-like domain refers to the full length mucin-like domain.

According to specific embodiments, the amino acid sequence of the mucin-like domain refers to a functional mucin-like domain.

As use herein, the phrase “functional mucin-like domain” refers to a portion of the mucin-like domain which maintains at least the L-selectin binding activities of the full length mucin-like domain. Methods of determining binding are well known in the art and are further described hereinabove.

According to specific embodiments, the amino acid sequence of the mucin-like domain corresponds to amino acids coordinates 221-257 of SEQ ID NO: 25.

According to specific embodiments, the amino acid sequence of the mucin-like domain corresponds to amino acids coordinates 226-319 of SEQ ID NO: 26.

According to specific embodiments, the mucin-like domain has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 39-40.

Besides containing the amino acid sequence of MAdCAM-1, the polypeptides of some embodiments of the present invention may further comprise additional elements to increase stability or penetration into cells, to aid in purification or in attachment to the therapeutic or the detectable moiety or a particle comprising same and/or to improve expression, pharmacokinetics, or bioactivity.

Non-limiting examples of such additional elements include polyethylene glycol (PEG), tags such as histidine tag, myc tag, flag tag, signal peptide and the like.

According to specific embodiments, the peptide further comprises an amino acid sequence of a constant region of an antibody.

According to specific embodiments the antibody constant region is chosen from, e.g., IgG1, IgG2, IgG3, IgG4, IgM, IgA1, IgA2, IgD, and IgE.

According to specific embodiments, the antibody constant region is of an IgG antibody.

According to a specific embodiment, the antibody constant region is of an IgG1, IgG2 or IgG4.

According to a specific embodiment, the antibody constant region is IgG2 e.g. IgG2a.

According to specific embodiments, the antibody constant region is of human origin.

As used herein, the phrase “amino acid sequence of a constant region of an antibody” refers to an amino acid sequence of the full length constant region of an antibody, or a fragment thereof. Hence, the constant region of an antibody may be a light chain constant region, a heavy chain constant region or a portion thereof e.g. an Fc domain or a portion thereof.

According to specific embodiments, the constant region of an antibody is an Fc domain of an antibody or a fragment thereof.

According to specific embodiments, the polypeptide comprises a portion of an Fc domain of an antibody excluding the CH1 domain.

According to a specific embodiments, the polypeptide comprises a portion of the Fc domain as set forth in SEQ ID NO: 13.

According to specific embodiments, the amino acid sequence of the antibody constant region is located C terminally to the amino acid sequence of MAdCAM-1.

The polypeptides of some embodiments of the invention or the proteinaceous moieties further described herein below may be synthesized by any techniques that are known to those skilled in the art of peptide synthesis, such as, but not limited to, solid phase and recombinant techniques.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

Large scale peptide synthesis is described by Andersson Biopolymers 2000; 55(3):227-50.

Alternatively or additionally, any of the polypeptides and proteinaceous moieties described herein can be encoded from a polynucleotide.

Thus, according to an aspect of the present invention there is provided an isolated polynucleotide encoding the polypeptide.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

To express exogenous peptide or agent in mammalian cells, a polynucleotide sequence encoding the peptide and/or the agent is preferably ligated into a nucleic acid construct suitable for mammalian cell expression.

Thus, according to an aspect of the present invention there is provided a nucleic acid construct comprising a polynucleotide encoding the polypeptide and a cis-acting regulatory element for directing expression of the polynucleotide in a cell.

Methods of recombinantly expressing a polypeptide are well known in the art.

As mentioned, the polypeptide is in association with a therapeutic moiety or a detectable moiety.

According to an embodiment, the polypeptide is attached to a therapeutic moiety or a detectable moiety.

As used herein, the phrase “in association with”, refers to direct or indirect (e.g. through a linker) binding of the polypeptide to the therapeutic or detectable moiety or to a particle comprising the therapeutic or detectable moiety.

According to specific embodiments, the therapeutic moiety or the detectable moiety is a heterologous therapeutic or detectable moiety.

The therapeutic moiety may be any molecule, including polynucleotides, small molecule chemical compounds and polypeptides.

Non-limiting examples of therapeutic moieties which can be used with specific embodiments of the invention include an anti-inflammatory cytokine (e.g IL4, IL-10, IL-13, IFNα, TGFβ), an anti-inflammatory drug, an immunosuppressive agent, steroids, an immunomodulatory agent, an enzyme.

According to specific embodiments, the therapeutic moiety is not a constant region of an antibody.

According to specific embodiments, the therapeutic moiety downregulates expression and/or activity of a gene of interest.

Downregulating expression and/or activity can be can be effected at the protein level (e.g., small molecules, inhibitory peptides, enzymes that cleave the polypeptide, aptamers and the like) or at the genomic (i.e. genome editing agent e.g. homologous recombination and site specific endonucleases) and/or the transcript level using a variety of molecules which interfere with transcription and/or translation (e.g., RNA silencing agents) of a target expression product described herein.

Non-limiting examples of downregulating agents are described in details hereinbelow.

Downregulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the therapeutic moiety is an RNA silencing agent.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

Non-limiting examples of RNA silencing agents that can be used according to specific embodiments of the present invention include dsRNA, siRNA, shRNA, antisense, miRNA and miRNA mimics. Methods and algorithms of designing RNA silencing agents and predicting their efficiency are well known in the art.

According to specific embodiments, the RNA silencing agent is a siRNA.

Nucleic acid agents can also operate at the DNA level as summarized infra.

Downregulation can also be achieved by inactivating the gene via introducing targeted mutations involving loss-of function alterations (e.g. point mutations, deletions and insertions) in the gene structure.

As used herein, the phrase “loss-of-function alterations” refers to any mutation in the DNA sequence of a gene which results in down-regulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene.

Methods of introducing nucleic acid alterations to a gene of interest are well known in the art using well known genome editing agents [see for example Menke D. Genesis (2013) 51: -618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination (e.g. “Hit and run”, “double-replacement”), site specific recombinases (e.g. the Cre recombinase and the Flp recombinase), PB transposases (e.g. Sleeping Beauty, piggyBac, Tol2 or Frog Prince), genome editing by engineered nucleases [e.g. meganucleases, Zinc finger nucleases (ZFNs), transcription-activator like effector nucleases (TALENs) and CRISPR/Cas system (including the gRNA and/or the endonuclease e.g. Cas9)] and genome editing using recombinant adeno-associated virus (rAAV) platform. Agents for introducing nucleic acid alterations to a gene of interest can be designed by publically available sources or obtained commercially from e.g. Transposagen, Addgene and Sangamo Biosciences. Thus, according to specific embodiments, the therapeutic moiety is a genome editing system or a component in a genome editing system, e.g. a homologous polynucleotide comprising a mutation, a gRNA, a restriction enzyme, a nuclease or a polynucleotide encoding same e.g. meganuclease, TALEN, ZFN, Cas9.

Downregulation can also be affected at the polypeptide level using e.g. small molecules, peptides, antibodies and/or polynucleotides.

An example of such a downregulating agent would be any molecule which interferes with the target protein activity (e.g., catalytic or interaction) by binding the target protein and/or cleaving the target protein. Such molecules can be a small molecule, antagonists, antibody or inhibitory peptide.

Another inhibitory agent which can be used along with some embodiments of the invention is a molecule which prevents target activation or substrate binding.

Another downregulating agent which can be used along with some embodiments of the invention is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

It will be appreciated that a non-functional analogue of at least a catalytic or binding portion of the target can be also used as an inhibitory agent.

According to specific embodiments, the therapeutic moiety downregulates expression and/or activity of a pro-inflammatory cytokine. Non-limiting examples of pro-inflammatory cytokines include IL-1, TNFα, IFNγ, IL-12, IL-18, granulocyte-macrophage colony stimulating factor.

According to specific embodiments, the therapeutic moiety downregulates expression and/or activity of IFNγ.

According to a specific embodiment, the therapeutic moiety is an IFNγ siRNA, such as, but not limited to SEQ ID NO: 9-10.

According to other specific embodiments, the therapeutic moiety is an anti-inflammatory cytokine or a polynucleotide encoding same. Non-limiting examples of anti-inflammatory cytokines include IL4, IL-10, IL-13, IFNα, TGFβ.

Examples of detectable moieties that can be used in the present invention include but are not limited to radioactive isotopes, phosphorescent chemicals, chemiluminescent chemicals, fluorescent chemicals, enzymes, fluorescent polypeptides and a radioactive isotope (such as ^{[125]}iodine). The detectable moiety can be a member of a binding pair, which is identifiable via its interaction with an additional member of the binding pair, and a label which is directly visualized. In one example, the label is a fluorescent protein or an enzyme producing a colorimetric reaction.

According to specific embodiments, the detectable moiety is not a tag such as histidine tag, myc tag, flag tag and the like which aid in purification of the polypeptide.

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the fluorescent detectable moiety include, for example, fluorescence activated flow cytometry (FACS), immunofluorescence confocal microscopy, fluorescence in-situ hybridization (FISH) and fluorescence resonance energy transfer (FRET).

Further examples of detectable moieties, include those detectable by Positron Emission Tomagraphy (PET) and Magnetic Resonance Imaging (MRI), all of which are well known to those of skill in the art.

According to specific embodiments, the therapeutic or detectable moiety is attached to or encapsulated in a cell-penetrating moiety.

As used herein the phrase “cell penetrating moiety” refers to a moiety which enhances translocation of the therapeutic or detectable moiety across a cell membrane. Non-limiting examples of cell penetrating moieties include cell penetrating peptides and lipid particles.

As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention may include, but are not limited to, penetratin, transportan, pIs1, TAT(48-60), pVEC, MTS, and MAP.

Additional description on lipid particles is provided hereinbelow.

According to specific embodiments, the therapeutic or detectable moiety is directly or indirectly bound to the polypeptide.

Accordingly, according to specific embodiments, the therapeutic or detectable moiety can be attached to the polypeptide covalently or non-covalently.

The therapeutic or detectable moiety can be attached to the polypeptide directly or via a linker.

According to specific embodiments, the therapeutic or detectable moiety is attached to the polypeptide via a linker. Any linker known in the art can be used with specific embodiments of the invention, including but not limited to a polypeptide, a synthetic linker, a chemical moiety, a polymer, a particle.

According to some embodiments, when the therapeutic or detectable moiety is proteinaceous it can be attached to the polypeptide by translationally fusing the polynucleotide encoding the polypeptide with the nucleic acid sequence encoding the therapeutic or detectable moiety.

Additionally or alternatively, the therapeutic or detectable moieties can be chemically conjugated (coupled) to the polypeptide, using any conjugation method known to one skilled in the art. For example, conjugation using a 3-(2-pyridyldithio)propionic acid N-hydroxysuccinimide ester (also called N-succinimidyl 3-(2pyridyldithio) propionate) (“SDPD”) (Sigma, Cat. No. P-3415; see e.g., Cumber et al. 1985, Methods of Enzymology 112: 207-224), a glutaraldehyde conjugation procedure (see e.g., G.T. Hermanson 1996, “Antibody Modification and Conjugation, in Bioconjugate Techniques, Academic Press, San Diego) or a carbodiimide conjugation procedure [see e.g., J. March, Advanced Organic Chemistry: Reaction's, Mechanism, and Structure, pp. 349-50 & 372-74 (3d ed.), 1985; B. Neises et al. 1978, Angew Chem., Int. Ed. Engl. 17:522; A. Hassner et al. 1978, Tetrahedron Lett. 4475; E. P. Boden et al. 1986, J. Org. Chem. 50:2394 and L. J. Mathias 1979, Synthesis 561].

A therapeutic or detectable moiety can be attached, for example, to the polypeptide using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

According to other embodiments, the therapeutic or detectable moiety is attached to-, or encapsulated in- a particle.

Accordingly, according to specific embodiments, the polypeptide is attached to the particle which comprises (also referred to as loaded with) the therapeutic or detectable moiety via direct or indirect binding.

As used herein, “particle” refers to a nano to micro structures which are not biological cells.

The particle may be a synthetic carrier, gel or other object or material having an external surface which is capable of being loadable with (e.g., encapsulated in or attached to) a therapeutic or detectable moiety. The particle may be either polymeric or non-polymeric preparations.

Exemplary particles that may be used according to specific embodiments of the invention include, but are not limited to, polymeric particles, microcapsules, liposomes, microspheres, microemulsions, nanoparticles, nanocapsules, nano-spheres, nano-liposomes, nano-emulsions, lipid nanoparticles and nanotubes.

In one embodiment, the particle is a biological particle—e.g. an erythrocyte or a cell ghost.

In another embodiment, the particle is a non-biological particle—i.e. not a cell.

Suitable particles in accordance with some embodiments of the invention are preferably non-toxic.

According to a particular embodiment, the particle is a nanoparticle.

As used herein, the term “nanoparticle” refers to a particle or particles having an intermediate size between individual atoms and macroscopic bulk solids. Generally, nanoparticle has a characteristic size (e.g., diameter for generally spherical nanoparticles, or length for generally elongated nanoparticles) in the sub-micrometer range, e.g., from about 1 nm to about 500 nm, or from about 1 nm to about 200 nm, or of the order of 10 nm, e.g., from about 1 nm to about 100 nm. According to specific embodiments, the particle is a nanoparticle having a size of 40-120 nm.

The nanoparticles may be of any shape, including, without limitation, elongated particle shapes, such as nanowires, or irregular shapes, in addition to more regular shapes, such as generally spherical, hexagonal and cubic nanoparticles. According to one embodiment, the nanoparticles are generally spherical.

The particles may have a charged surface (i.e., positively charged or negatively charged) or a neutral surface.

Agents which are used to fabricate the particles may be selected according to the desired charge required on the outer surface of the particles.

According to specific embodiments, the particle if a lipid particle.

Thus, for example if a negatively charged surface is desired, the particles may be fabricated from negatively charged lipids, such as described herein below.

When a positively charged surface is desired, the particles may be fabricated from positively charged lipids, such as described herein below.

As mentioned, non-charged particles are also contemplated by the present invention. Such particles may be fabricated from neutral lipids such as phosphatidylethanolamine or dioleilphosphatidylethanolamine (DOPE).

In a specific embodiment, the particle is a liposome. As used herein and as recognized in the art, liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D., Liposomes Technology Inc., Elsevier, 1993, 63-105. (chapter 3); Winterhalter M, Lasic D, Chem Phys Lipids, 1993 September; 64(1-3):35-43].

The diameter of the liposomes used preferably ranges from 20-200 nm and more preferably from 20-100 nm. For sizing liposomes, extrusion, homogenization or exposure to ultrasound irradiation may be used, Homogenizers which may be conveniently used include microfluidizers (produced by Microfluidics of Boston, MA, USA) or microfluidic micro mixer (Precision NanoSystems, Vancouver, BC, Canada). In a typical homogenization procedure, liposomes are recirculated through a standard emulsion homogenizer until selected liposomes sizes are observed. The particle size distribution can be monitored by conventional laser beam particle size discrimination. Extrusion of liposomes through a small-pore polycarbonate membrane or an asymmetric ceramic membrane is an effective method for reducing liposome sizes to a relatively well defined size distribution. Typically, the suspension is cycled through the membrane one or more times until the desired liposome size distribution is achieved. The liposomes may be extruded through successively smaller pore membranes to achieve a gradual reduction in liposome size.

The liposomes may be unilamellar or may be multilamellar. Unilamellar liposomes may be preferred in some instances as they represent a larger surface area per lipid mass. The liposomes may be fabricated from a single phospholipid or mixtures of phospholipids. The liposomes may also comprise other lipid materials such as cholesterol. For fabricating liposomes with a negative electrical surface potential, acidic phospho- or sphingo- or other synthetic-lipids may be used. Preferably, the lipids have a high partition coefficient into lipid bilayers and a low desorption rate from the lipid assembly. Exemplary phospholipids that may be used for fabricating liposomes with a negative electrical surface potential include, but are not limited to phosphatidylserine, phosphatidic acid, phosphatidylcholine and phosphatidyl glycerol.

Other negatively charged lipids which are not liposome forming lipids that may be used are sphingolipids such as cerebroside sulfate, and various gangliosides.

The most commonly used and commercially available lipids derivatized into lipopolymers are those based on phosphatidyl ethanolamine (PE), usually distearylphosphatidylethanolamine (DSPE).

The lipid phase of the liposome may comprise a physiologically acceptable liposome forming lipid or a combination of physiologically acceptable liposome forming lipids for medical or veterinarian applications. Liposomes are spherical bilayer structures composed of different kind of amphiphatic lipids. These lipids have glycerol backbone with two hydrophobic acyl chains and a hydrophilic head groups such as phosphate in combination or derivatives of the same may contain amine, imine, acid or alcohols.

Typically, the acyl chain is between 12 to about 24 carbon atoms in length, and has varying degrees of saturation being fully, partially or non-hydrogenated lipids. Further, the lipid matrix may be of natural source, semi-synthetic or fully synthetic lipid, and neutral, negatively or positively charged.

According to specific embodiments, the particle is a lipid nanoparticle.

Non-limiting examples of lipid nanoparticles that can be used with specific embodiments of the present invention and methods of producing same are further described hereinbelow and in the Examples section which follows, and also in e.g. Ramishetti et al. Adv Mater. 2020 Jan. 30:e1906128, International Patent Application publication Nos. WO2016/189532, WO2018/015881 and WO2018087753, WO2017194454 and US Patent Application Publication no. US20130245107, the contents of which are fully incorporated herein by reference.

The lipid nanoparticle may be prepared by any of the methods known in the art, such as disclosed in e.g. Jayaraman et al. Angew chem. July 2012, Semple et al. Nat Biotech. 2010, Kauffman et al. Nano Lett, Oct 2015; and in the Examples section which follows.

The core of the particle may be hydrophilic or hydrophobic. The core of the lipid nanoparticle may comprise some lipids, such that it is not fully hydrophilic.

According to specific embodiments, the core of the particle is hydrophobic.

According to specific embodiments, the core of the particle is hydrophilic.

According to specific embodiments, the core of the particle contains an amorphous lipid core.

It will be appreciated that combinations of different lipids may be used to fabricate the particles disclosed herein, including a mixture of more than one cationic lipid, a mixture of more than one anionic lipid, a mixture of more than one neutral lipid, a mixture of more than one ionizable lipid, a mixture of at least one cationic lipid and at least one anionic lipid, a mixture of at least one cationic lipid and at least one neutral lipid, a mixture of at least one anionic lipid and at least one neutral lipid, a mixture of at least one ionizable lipid and at least one neutral lipid, and additional combinations of the above.

According to some exemplary embodiments, the plurality of lipids of the lipid particles may be of natural or synthetic source and may be selected from, but not limited to: cationic lipids, phosphatidylethanolamines, ionizable lipids, membrane stabilizing lipids, phospholipids, and the like, or combinations thereof. Each possibility represents a separate embodiment of the present invention.

In some embodiments, the membrane stabilizing lipids may be selected from, but not limited to: cholesterol, phospholipids (such as, for example, phosphatidylcholine, phosphatidylethanolamine, phosphatidylinositol, phosphatidylserine, phosphatidylglycerol, diphosphatidylglycerols), cephalins, sphingolipids (sphingomyelins and glycosphingolipids), glycoglycerolipids, and combinations thereof. Each possibility represents a separate embodiment of the present invention.

In some embodiments, the phosphatidylethanolamines may be selected from, but not limited to: 1,2-dilauroyl-L-phosphatidyl-ethanolamine (DLPE), 1,2-Dioleoyl-sn-glycero-3-phosphoethanolamine (DOPE), 1,2-Diphytanoyl-sn-glycero-3-phosphoethanolamine (DPhPE) 1,3 -Dipalmitoyl-sn-glycero-2-phosphoethanolamine (1,3 -DPPE), 1-Palmitoyl-3 -oleoyl-sn-glycero-2-phosphoethanolamine (1,3-POPE), Biotin-Phosphatidylethanolamine, 1,2-Dimyristoyl-sn-glycero-3-phosphoethanolamine (DMPE), Dipalmitoylphosphatidylethanolamine (DPPE), 1,2-Distearoyl-sn-glycero-3-phosphoethanolamine (DSPE) or combinations thereof. In some embodiments, the Phosphatidylethanolamines may be conjugated to a PEG-Amine derivative. Each possibility represents a separate embodiment of the present invention.

In addition, according to specific embodiments, polymer-lipid based formulations may be used.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactie-polyglycolic acid′ polyvinyl alcohol, polyvinylpyrrolidone, polymethoxazoline, polyethyloxazoline, polyllydroxyetlyloxazolille, solyhydroxypryloxazoline, polyaspartarllide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

The polymers may be employed as homopolymers or as block or random copolymers.

The particles may also include other components. Examples of such other components includes, without being limited thereto, fatty alcohols, fatty acids, and/or cholesterol esters or any other pharmaceutically acceptable excipients which may affect the surface charge, the membrane fluidity and assist in the incorporation of the biologically active lipid into the lipid assembly. Examples of sterols include cholesterol, cholesterol hemisuccinate, cholesterol sulfate, or any other derivatives of cholesterol. Lipid assemblies according to specific embodiments of the invention include either those which form a micelle (typically when the assembly is absent from a lipid matrix) or those which form a liposome (typically, when a lipid matrix is present).

According to one embodiment, the lipid phase comprises phospholipids.

The phospholipids may be a glycerophospholipid. Examples of glycerophospholipid include, without being limited thereto, phosphatidylglycerol (PG) including dimyristoyl phosphatidylglycerol (DMPG); phosphatidylcholine (PC), including egg yolk phosphatidylcholine and dimyristoyl phosphatidylcholine (DMPC), phosphatidic acid (PA), phosphatidylinositol (PI), phosphatidylserine (PS) and sphingomyelin (SM) and derivatives of the same.

Another group of lipid matrix employed according to the invention includes cationic lipids (monocationic or polycationic lipids). Cationic lipids typically comprise a lipophilic moiety, such as a sterol or the same glycerol backbone to which two acyl or two alkyl, or one acyl and one alkyl chain contribute the hydrophobic region of the amphipathic molecule, to form a lipid having an overall net positive charge. Non-limiting examples of cationic lipids that may be used with specific embodiments of the invention include, 1,2-dimyristoyl-3-trimethylammonium propane (DMTAP) 1,2-dioleyloxy-3-(trimethylanino) propane (DOTAP), N-[-1-(2,3,-ditetradecyloxy)propyl]-N,N-dimethyl-N-hydroxyethylammonium bromide (DMRIE), N-[1-(2,3,-dioleyloxy)propyl]-N,N-dimethyl-N-hydroxy ethyl-ammonium bromide (DORIE), N-[1-(2,3-dioleyloxy)propyl];-N,N,N-trimethylammonium chloride (DOTMA); 3 ;N-(N′,N′- dimethylaminoethane) carbamoly]; cholesterol (DC-Chol), and I dimethyldioctadecylammonium (DDAB), N-[2-[[2,5-bis[3aminopropyl)amino]-1-oxopentyl]amino ]ethyl]N,N dimethul-2,3 bis (1-oXo-9-octadecenyl) oX;-1 propanaminium (DOSPA), ceramide carbamoyl spermine (CCS), D-Lin-MC3-DMA (Cas No. 1224606-06-7) and the cationic lipids described in Ramishetti et al. Adv Mater. 2020 Jan. 30:e1906128 and International Application Publication No. WO201808775, the contents of which are fully incorporated herein by reference.

According to a specific embodiments, the cationic lipid is D-Lin-MC3-DMA.

The cationic lipids may be used alone, in combination with cholesterol, with neutral phospholipids or other known lipid assembly components. In addition, the cationic lipids may form part of a derivatized phospholipids such as the neutral lipid dioleoylphosphatidyl ethanolamine (DOPE) derivatized with polylysine to form a cationic lipopolymer.

According to specific embodiments, the polymer used for fabricating the particles is biocompatible and biodegradable, such as poly(DL-lactide-co-glycolide) polymer (PLGA). However, additional polymers which may be used for fabricating the particles include, but are not limited to, PLA (polylactic acid), and their copolymers, polyanhydrides, polyalkyl-cyanoacrylates (such as polyisobutylcyanoacrylate), polyethyleneglycols, polyethyleneoxides and their derivatives, chitosan, albumin, gelatin and the like.

The particles of the present invention may be modified to enhance their circulatory half-life (e.g. by PEGylation) to reduce their clearance, to prolong their scavenging time-frame and to allow antibody binding. The PEG which is incorporated into the particles may be characterized by of any of various combinations of chemical composition and/or molecular weight, depending on the application and purpose.

According to some embodiments, the particle include one or more PEG derivatives. According to specific embodiments, the PEG or PEG derivative may be conjugated to as, a lipid. Non-limiting examples of PEG derivative include PEG-DMG 3-N-(-methoxy poly(ethylene glycol)2000)carbamoyl-1,2-dimyrisyl glycerol, PEG-cDMA 3-N-(-methoxy poly(ethylene glycol)2000)carbamoyl-1,2-dimyristyloxy-propylamine; PEG-cDSA, 3-N-(-methoxy poly(ethylene glycol)2000)carbamoyl-1,2-distearyloxy-propylamine, DSPE-PEG, PEG-maleimide, DSPE-PEG-maleimide, or combinations thereof.

According to some embodiments, the lipid phase may comprise about 30-60% (mol) cationic lipids. For example, the cationic lipid(s) may comprise about 40-50% (mol) of the lipid phase.

According to some embodiments, the lipid phase may comprise about 20-70% (mol) membrane stabilizing lipids. For example, the membrane stabilizing lipids may comprise about 40-60% of the lipid phase. In some embodiments, more than one type of membrane stabilizing lipid may be used in the lipid phase. For example, the membrane stabilizing lipid may include cholesterol (being about 30-50% (mol) of the lipid phase), and a phospholipid (such as, for example, DSPC), that may be about 5-15% (mol) of the lipid phase.

According to some embodiments, the lipid phase may comprise about 0.01-3% (mol) of PEG-maleimide (optionally conjugated to a lipid). For example, the PEG-maleimide may comprise about 0.05-0.6% of the lipid mixture.

According to some embodiments, an additional PEG-derivative (conjugated to a lipid) may comprise about 0.5-10% of the lipid phase composition.

According to exemplary embodiments, the particles may be comprised of a cationic lipid (such D-Lin-MC3-DMA), cholesterol, 1,2-Distearoyl-sn-glycero-3-phosphocholine (DSPC), PEG derivative (such as DMG-PEG) and PEG-maleimide conjugated to a lipid (such as DSPE-PEG-maleimide); at various mol/mol ratios. For example, the lipid phase may be comprised of: cationic lipid (DLinMC3-DMA)/Chol/DSPC/DMG-PEG/DSPE-PEG-maleimide (mol/mol 50:10:38:1.5:0.5).

Attaching the therapeutic or detectable moiety to the particle can be effected concomitant with, or following particle assembly, by methods well known in the art such as disclosed in the Examples section which follows, and also in e.g. Ramishetti et al. Adv Mater. 2020 Jan. 30:e1906128, International Patent Application publication Nos. WO2018/015881 and WO2018087753, WO2017194454 and US Patent Application Publication no. US20130245107, the contents of which are fully incorporated herein by reference.

Any suitable particle (e.g. lipid particle): therapeutic or detectable moiety ratio that is efficacious is contemplated by some embodiments of the invention. According to specific embodiments, the particle (e.g. lipid particle): therapeutic or detectable moiety ratios (w/w) include about 1:1 to about 50:1, about 2:1 to about 30:1, about 5:1 to about 100:1, about 10:1 to about 40:1, about 15:1 to about 25:1. According to a specific embodiment the particle (e.g. lipid particle) : therapeutic or detectable moiety ratios (w/w) is 16:1.

The desired amount of the therapeutic or detectable moiety attached to- or encapsulated in the particle varies depending on the type of the therapeutic or detectable moiety. However, it is preferable that the therapeutic or detectable moiety can be attached to- or encapsulated in the particle at a high loading efficiency.

Methods of covalently binding a polypeptide to a particle are known in the art and disclosed in the Examples section which follows and for example in U.S. Pat. Nos. 5,171,578, 5,204,096 and 5,258,499, the contents of which are fully incorporated herein by reference. Methods of non-covalently binding a polypeptide to a particle are also known in the art and are also disclosed in the Examples section which follows and e.g. International Patent Application Publication NO. WO2018/015881, the contents of which are fully incorporated herein by reference.

According to a specific embodiment, the polypeptide is attached to the particle directly or via a linker.

According to specific embodiments, the polypeptide binds the particle via a linker. The linker may be attached to the particle and/or the polypeptide covalently or non-covalently.

According to specific embodiments, the linker is covalently attached to the particle.

According to specific embodiments, the linker is chemically conjugated (coupled) to the particle. Such methods are known in the art and are further described hereinabove and in the Examples section which follows. According to a specific embodiment, the linker is chemically conjugated to the particle using maleimide-thiol chemistry.

According to specific embodiments, the linker is non-covalently attached to the polypeptide.

Linkers that can be used with specific embodiments of the invention are known in the art and are further described hereinabove and below.

According to specific embodiments, the linker is a protein capable of forming an immune-complex with the polypeptide. Thus, according to specific embodiments, the polypeptide binds the particle via a protein capable of immune-complexing with an antibody constant region or a tag comprised in the polypeptide. Non-limiting examples of such proteins include a secondary anti-FC antibody or an anti-tag antibody (e.g. ScFv), Protein A, Protein G, Protein L, Protein Z, Protein LG, Protein LA and Protein AG, proteins described in Lombardi et al., Discovery Today, Volume 20, Number 10, Pages 1271-1283, October 201; Braisted et al., Proc. Natl. Acad. Sci. USA, Vol. 93, pp. 5688-5692, June 1996; and Y. J. Jeong et al., Peptides 31 (2010) 202-206, the contents of which are incorporated herein by reference.

According to a specific embodiments, the polypeptide binds the particle via a secondary antibody that binds the antibody constant region comprised in the polypeptide.

According to specific embodiments, the antibody is a ScFv.

According to specific embodiment, the antibody is a humanized antibody.

Thus, according to an aspect of the present invention there is provided a method for the preparation of a targeted particle, the method comprising attaching a therapeutic or a detectable moiety to a particle or encapsulating therapeutic or a detectable moiety in a particle and attaching the loaded particle to the polypeptide comprising an amino acid sequence of MAdCAM-1 disclosed herein to generate a targeted particle.

According to an aspect of the present invention, there is provided a targeted particle comprising a therapeutic and/or a detectable moiety, said particle is attached to a polypeptide comprising an amino acid sequence of MAdCAM-1 disclosed herein.

According to specific embodiments, the therapeutic or a detectable moiety is attached to said particle.

According to specific embodiments, the therapeutic or a detectable moiety is encapsulated in said particle.

As the polypeptides comprising an amino acid sequence of MAdCAM-1 and targeted particles disclosed herein are in association with a therapeutic or a detectable moieties, specific embodiments of the present invention contemplate their use in methods of treating, diagnosing and monitoring treatment efficacy in conditions associated with inflammation mediated by MAdCAM-1.

Thus, according to an aspect of the present invention, there is provided a method of treating a disease associated with inflammation mediated by MAdCAM-1 in a subject in need thereof, the method comprising administering to the subject a therapeutically effective amount of the polypeptide, thereby treating the disease associated with inflammation mediated by MAdCAM-1.

According to an additional or an alternative aspect of the present invention, there is provided the polypeptide for use in treating a disease associated with inflammation mediated by MAdCAM-1.

According to an additional or an alternative aspect of the present invention, there is provided method of treating a disease associated with inflammation mediated by MAdCAM-1 in a subject in need thereof, the method comprising administering to the subject a therapeutically effective amount of the targeted particle, thereby treating the disease associated with inflammation mediated by MAdCAM-1.

According to an additional or an alternative aspect of the present invention, there is provided the targeted particle for use in treating a disease associated with inflammation mediated by MAdCAM-1.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, e.g., human beings at any age and of any gender. According to specific embodiments, the term “subject” refers to a subject who suffers from the pathology (disease, disorder or medical condition).

According to specific embodiments, the subject is a human.

As used herein the phrase “inflammation mediated by MAdCAM-1” refers to pathological condition which involves inflammation in which MAdCAM-1 activity contributes to onset or progression.

As used herein, the phrase “disease associated with inflammation mediated by MAdCAM-1” refers to a disease in which inflammation mediated by MAdCAM-1 contributes to onset or progression.

Non-limiting examples of diseases associated with inflammation mediated by MAdCAM-1 include inflammatory bowel disease (IBD), such as ulcerative colitis, Crohn's disease, ileitis, Celiac disease, nontropical Sprue, enteropathy associated with seronegative arthropathies, microscopic or collagenous colitis, eosinophilic gastroenteritis, or pouchitis resulting after proctocolectomy, and ileoanal anastomosis, pancreatitis and insulin-dependent diabetes mellitus, mastitis (mammary gland), cholecystitis, cholangitis or pericholangitis (bile duct and surrounding tissue of the liver), chronic bronchitis, chronic sinusitis, asthma, and graft versus host disease (e.g., in the gastrointestinal tract), chronic inflammatory diseases of the lung which result in interstitial fibrosis, such as hypersensitivity pneumonitis, collagen diseases, sarcoidosis, autoimmune-mediated liver disease and other idiopathic conditions.

According to specific embodiments, the disease is IBD.

According to an additional or an alternative aspect of the present invention, there is provided a method of diagnosing inflammation mediated by MAdCAM-1 in a subject in need, the method comprising:
(a) administering to the subject the polypeptide or the targeted particle; and
(b) determining an amount of said polypeptide or said targeted particle in a suspected inflamed tissue of the subject following said administering,
   wherein an amount above a predetermined threshold is indicative of presence of inflammation mediated by MAdCAM-1 in said tissue of said subject.

According to an additional or an alternative aspect of the present invention, there is provided a method of monitoring efficacy of treatment against inflammation mediated by MAdCAM-1 in a subject in need, the method comprising:
(a) administering to the subject the polypeptide or the targeted particle following the treatment; and
(b) determining an amount of the polypeptide or the targeted particle in a suspected inflamed tissue of the subject following said administering,
   wherein a decrease in the amount following said treatment beyond a predetermined threshold is indicative of reduction in inflammation and efficaciousness of the treatment.

According to specific embodiments, the tissue is a gut tissue.

Determining the amount of the polypeptide or the targeted particle may be effected by any method known in the art and depends on the detectable moiety used. Non-limiting examples of such methods include PET/CT and in-vivo fluoresce imaging.

According to specific embodiments, the inflammation is associated with a disease, such as the ones disclosed hereinabove e.g. IBD.

Thus, according to specific embodiments, the method comprising treating the subject with said treatment prior to administration of the polypeptide or the targeted particle.

Non-limiting Examples of treatments include anti-inflammatory drugs (such as, corticosteroids and sulfasalazine), immunosuppressive drugs (such as, 6-mercaptopurine, cyclosporine and azathioprine) and anti-TNFα.

According to specific embodiments, treatment is with the polypeptide comprising the MAdCAM-1 amino acid sequence attached to a therapeutic moiety or the targeted particle described herein.

According to specific embodiments, the predetermined threshold is derived from a control subject, such as a healthy subject or a subject with a known disease.

Thus, the predetermined level can be experimentally determined by comparing the amount in a healthy subject with the amount in a subject having an inflammatory disease mediated by MAdCAM-1 (e.g. IBD) with known stage.

According to specific embodiments, the decrease beyond a predetermined threshold is statistically significant.

The polypeptide or targeted particle of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide in association with a therapeutic or detectable moiety accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., acute liver disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The polypeptide attached to a therapeutic moiety or targeted particle of some embodiments of the invention can be administered to the subject as a single treatment or in combination with other established or experimental therapeutic regimen to treat an inflammatory disease mediated by MAdCAM-1 (e.g. IBD) (e.g., before, simultaneously or following) including, but not limited to anti-inflammatory drugs (such as, corticosteroids and sulfasalazine), immunosuppressive drugs (such as, 6-mercaptopurine, cyclosporine and azathioprine), anti-TNFα, surgery (such as colectomy) and other treatment regimens known in the art.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Antibodies—Rat anti-mouse α₄β₇ integrin (Clone DATK32, Biolegend), Rat anti-mouse β₇ integrin (Clone FIB504, BioXCell), Mouse anti-rat IgG₂ₐ (Clone RG7/1.30, BioXCell), Rat IgG₂ₐ isotype control (Clone 2A3, BioXCell), PE-conjugated donkey anti-mouse IgG (Polyclonal, Jackson Immuno Research), AlexaFluor 647-conjugated goat anti-rat IgG (Polyclonal, Biolegend), AlexaFluor 647-conjugated mouse anti-human IgG (Polyclonal, Biolegend), AlexaFluor 647-conjugated rat anti-mouse CD45 (Clone 30-F11, Biolegend), AlexaFluor 488-conjugated rat anti-mouse CD45 (Clone 30-F11, Biolegend), BV650-conjugated rat anti-mouse/human CD11b (Clone M1/70, Biolegend), PE/Cy5-conjugated rat anti-mouse CD19 (Clone 6D5, Biolegend), BV421-conjugated hamster anti-mouse CD3ε(Clone 145-2C11, Biolegend), APC/Cy7-conjugated rat anti-mouse CD4 (Clone GK1.5, Biolegend), AlexaFluor 488-conjugated rat anti-mouse CD8a (Clone 53-6.7, Biolegend), PE-conjugated rat anti-mouse CD8a (Clone 53-6.7, Biolegend), PE/Dazzle 594 rat anti-mouse CD25 (Clone PC61, Biolegend), BV510 armenian hamster anti-mouse CD69 (Clone H1.2F3, Biolegend), PE-conjugated rat anti-mouse FOXP3 (Clone MF-14, Biolegend), HRP-conjugated donkey anti-rat IgG (Polyclonal, Jackson Immuno Research).

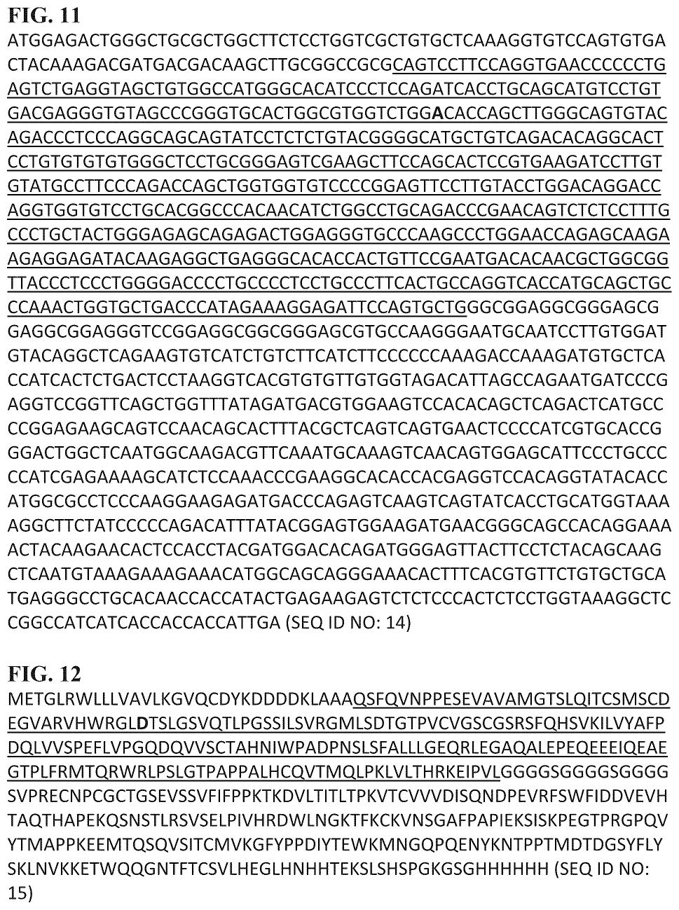

Cloning—Sequences of the primers used are listed in Table 1 hereinbelow. Murine MAdCAM-1 D1 and D2 domains (hereinafter “D1D2”) were synthesized as a gBlock gene fragment (Integrated DNA Technologies, USA) and cDNA of Rat IgG₂ₐ was obtained from an in-house hybridoma clone using RNeasy minkit (Qiagen, Netherlands) and qScript cDNA synthesis kit (Quantabio, USA). The Fc of Rat IgG₂ₐ was amplified from the obtained cDNA using primer pair F1 and R1, the primers added the required homology sequences for the Gibson assembly to the 5′ ends of the amplicon. Following, the murine MAdCAM-D1D2 and IgG Fc were assembled in the pCMV3-FLAG plasmid using Gibson Assembly (New England Biolabs, USA). In a later stage, the entire construct was reassembled in the pcDNA3.4 expression plasmid to improve protein yield using primer pair F2 and R2. The point mutation for the negative control, hereinafter “mD1D2”, was generated using site-directed mutagenesis with primers F3 and R3 used to amplify the entire plasmid while generating the point mutation. The resulting PCR product was DpnI digested, purified on a 0.8% agarose gel, phosphorylated using T4 Polynucleotide kinase (New England Biolabs, USA), circularized using T4 DNA Ligase (New England Biolabs, USA) and transformed into chemically competent bacteria. To improve flexibility in the context of the LNPs, the C_{H}1 domain of Rat IgG₂ₐ was later removed by PCR amplification of the entire plasmid using primers F4 and R4. See FIG. 11 for the sequence of the entire construct.

TABLE 1
Primers used for cloning of the
MAdCAM-D1D2-Fc construct.

| | | | SEQ |
|---|---|---|---|
| Primer | Function | Sequence (5′ → 3′) | ID NO |
| F1 | Amplifies Rat | GAAAACCTGTACTTTCAGGGCGC | 1 |
| | IgG₂ₐ-Fc | TGAAACAACAGCCCCATCTGTC | |
| R1 | Amplifies Rat | CCTCTAGAGTCGACTGGTACCGA | 2 |
| | IgG₂ₐ-Fc | TATCAGATCTATCGATGTCATTT | |
| | | ACCAGGAGAGTGGGAGAG | |
| F2 | Cloning into | CTGGTCGCTGTGCTCAAAGGTGT | 3 |
| | pcDNA3.4 | CCAGTGTGACTACAAAGACGATG | |
| | | ACGAC | |
| R2 | Cloning into | GATCGAACCCTTGTCGAGGTCGG | 4 |
| | pcDNA3.4 | GGGATCCTCATTTACCAGGAGAG | |
| | | TGGG | |
| F3 | Site-directed | GTGCACTGGCGTGGTCTGGCCAC | 5 |
| | mutagenesis | CAGCTTGGGCAGTGTAC | |
| R3 | Site-directed | GTACACTGCCCAAGCTGGTGGCC | 6 |
| | mutagenesis | AGACCACGCCAGTGCAC | |
| F4 | Removes C_{H}1 | GAAAACCTGTACTTTCAGGGCGG | 7 |
| | domain | TTCTGGTGTGCCAAGGGAATGCA | |
| | | ATCCTTGTGGATGT | |
| R4 | Removes C_{H}1 | ACCAGAACCACCTAGTACTGGAA | 8 |
| | domain | TCTCCTTTCTATGGGTCAGCACC | |
| | | AG | |

Cell Culture—Cell lines used: Expi293 (ThermoFisher Scientific), TK-1 (ATCC), HEK293 (ATCC). All cell lines were tested every 2 months for mycoplasma and discarded when positive. Expi293 cells were grown in Expi293 Expression Medium (ThermoFisher Scientific, USA) in disposable Erlenmeyer flasks at 37° C. and 8% CO₂ on a shaker rotating at 125 rpm. Cells were grown at densities between 0.3×10⁶-5×10⁶ cells per ml. At least 3 passages after thawing, cells were transfected with the expression plasmid encoding the D1D2-Fc using the Expifectamine293 transfection kit (ThermoFisher Scientific, USA). 18 hours post transfection, enhancer 1 and enhancer 2 from the Expifectamine293 transfection kit were added to boost recombinant protein expression levels. Five days post transfection the culture medium was harvested for purification of the secreted protein.

TK-1 cells were grown in RPMI1640 medium supplemented with 10% fetal bovine serum, L-glutamine and Pen-Strep-Nystatin (Biological Industries, Israel). Cells were grown at densities between 0.3×10⁶-2×10⁶ cells per ml in either T25 or T75 cell culture flasks (Greiner bio-one, Austria).

Protein purification—Conditioned medium was separated from the Expi293 cells by centrifugation at 300 g for five minutes. The supernatant was centrifuged again at 5000 g for 20 minutes to remove cellular debris. Using the Äkta FPLC protein purification system (GE Healthcare, UK), the protein was purified with a 1 ml Histrap column (GE Healthcare, UK). The sample was adjusted to the composition of the binding buffer (20 mM NaPO₄ at pH 7.4 and 500 mM NaCl) and passed through a 0.2 μm syringe filter (Sartorius, Germany) prior to loading into the FPLC. Flow rate during binding and elution was 0.5 ml/min and during washes 1 ml/min. The columns were washed with 20 ml of 0.5 M NaOH and 20 ml deionized water before it was equilibrated with 10 ml of binding buffer and loaded with the sample. Protein elution was effected using binding buffer supplemented with 0.5 M imidazole. The elution was performed stepwise with incremental increases of ˜30 mM imidazole per fraction until a final concentration of 0.5 M was reached. Following, fractions were loaded on SDS-PAGE gel and stained with Coomassie to determine which fractions contained the D1D2-Fc at sufficient purity. Pooled fractions were buffer exchanged to PBS using PD-10 desalting columns (GE Healthcare, UK). The purified protein was concentrated to >1 mg/ml using Amicon ultra centrifugal filters (EMD Millipore, USA), snap frozen in liquid nitrogen and stored at −80° C.

SDS-PAGE gel analysis—As the presence of the hinge domain should dimerize the D1D2, protein sample was diluted in sample buffer with or without the presence of 500 mM DTT to compare between monomers and dimers. Samples were loaded on a 10% polyacrylamide gel and either stained with Coomassie (Bio-rad, USA) or transferred to a nitrocellulose membrane using iBlot 2 Dry Blotting System (ThermoFisher Scientific, USA). Membrane was blocked with 5% skim milk in PBS for 2 hours at RT and incubated with anti-rat IgG-HRP (Jackson Immunoresearch, USA). Following, membrane was washed with PBS+0.01% Tween20 and developed using SuperSignal West Pico chemiluminescent substrate (ThermoFisher Scientific, USA). Chemiluminescence was measured with the Amersham Imager 600 (GE Healthcare, USA).

In-vitro binding—TK-1 cells were activated according to Y. Yang et al.³⁰. Briefly, cells were washed with PBS and resuspended in resuspension buffer (HBSS with 10 mM HEPES buffer, 2 mM CaCl₂ and 2 mM MgCl₂). Non-activated cells were kept on ice while activated cells were resuspended in pre-incubation buffer (HBSS with 10 mM HEPES buffer and 2 mM EDTA), incubated at RT for 30 minutes with gentle rotation, washed with PBS and finally resuspended in activation buffer (HBSS with 10 mM HEPES buffer, 2 mM CaCl₂ and 2 mM MnCl₂). MAdCAM-D1D2-Fc and the controls were added to both activated and non-activated cells and incubated for 30 minutes at 4° C. Cells were subsequently washed and stained with either anti-human IgG or anti-mouse IgG conjugated to AlexaFluor647 (Biolegend, USA). Binding of D1D2-Fc to TK-1 cells was assessed by analyzing the fluorescence of the cells by flow cytometry. Binding of D1D2-targeted Cy5-LNPs was detected by flow cytometry directly using the Cy5 fluorescence (without addition of another antibody). In the binding experiments with Cy5-LNPs, the HBSS in the activation buffer was replaced with RPMI+10% FBS due to a high background using LNPs.

Preparation of LNPs—LNPs were prepared as previously described ³¹ by using the Nanoassemblr microfluidic mixer (Precision Nanosystems, Canada). The current gold standard for LNP production, DLin-MC3-DMA, was used as an ionizable lipid with a pKₐ of 6.44 that obtains a positive charge under acidic conditions. LNPs were prepared at pH 4.5 to ensure that DLin-MC3-DMA is ionized and hence siRNA encapsulation is maximized. Lipid mixture (DLin-MC3-DMA, DSPC, Cholesterol, PEG-DMG and DSPE-PEG-Maleimide at 50:10:38:1.5:0.5 molar ratio) in ethanol was mixed with siRNA in acetate buffer, pH 4.5, at a combined flowrate of 2 ml/min. Lipid and siRNA were mixed at a 1:3 volume ratio (1:16 w/w siRNA to lipid). For Cy5-labeled LNPs, 20% Cy5-labeled siRNA (SEQ ID NO: 9-10) was mixed with 80% unlabeled siRNA. The resulting LNPs were dialyzed against PBS for 24 hours to remove the ethanol and restore the pH to neutral. The hydrodynamic diameter and Zeta potential of the LNPs were measured by dynamic light scattering using disposable cuvettes in the Malvern Zetasizer (Malvern Instruments, UK).

Determining the siRNA encapsulation efficiency—LNPs were either lysed with Triton X-100 or not and the total amount of siRNA in the sample was measured with the Quant-iT Ribogreen RNA assay kit (ThermoFisher Scientific, USA). After subtracting the blank measurement, the encapsulation efficiency (in percentage) was calculated by (1-Non-lysed LNPs/Lysed LNPs)*100.

Conjugation of RG7 to LNPs and gel filtration—RG7/1.3 antibody (BioXCell, USA) was reduced in PBS supplemented with 1 mM DTT and 5 mM EDTA by incubating for 1 hour at room temperature. The DTT was subsequently removed by buffer exchange to 5 mM EDTA in PBS using 7K Zeba spin desalting columns (ThermoFisher Scientific, USA). Immediately following buffer exchange, the reduced antibody was added to the LNPs at a ratio of 0.67 mg antibody per ml of LNPs. The mixture was incubated for 2 hours at room temperature with gentle shaking followed by overnight incubation at 4° C. The following day, LNPs were separated from the free antibody using sepharose CL4B beads on a gel filtration column with PBS as the mobile phase. Fractions containing pure LNPs were pooled and concentrated to the initial volume using 100K Amicon centrifugal filters (EMD Millipore, USA). The loss of LNPs during conjugation and gel filtration was estimated by lysing LNPs prior to and following conjugation with Triton X-100 followed by measuring the amount of released siRNA using Quant-iT Ribogreen RNA assay kit (ThermoFisher Scientific, USA). Following to attach the generated MAdCAM-D1D2-Fc or MAdCAM-mD1D2-Fc fusion proteins or a DATK32 antibody (BioXcell, cat no: BE0034), the RG7-LNPs were incubated with the fusion protein or the antibody for 30 minutes at room temperature.

Dot blot analysis—Dot blot analysis was performed using Minifold I system 96-wells device (GE Healthcare, UK). A nitrocellulose membrane was added on top of two filter papers (Whatman plc, UK) and loaded into the 96 dot blot device. Wells were filled with PBS and vacuum was applied to wet the membrane. Samples (conjugated LNPs, unconjugated LNPs and several different amounts of free antibody) were added to the wells in a 100 μl volume followed by a vacuum to pass the sample through the membrane. Wells were washed with PBS and the device was again applied to a vacuum. Following, the membrane was blocked with 5% skim milk in PBS for 2 hours followed by incubation with anti-mouse IgG antibody linked to HRP (diluted in PBS with 1% skim milk) for 1 hour at room temperature. Next, the membrane was washed 3 times with PBST (5 minutes per wash) and the samples were detected by adding the SuperSignal West Pico chemiluminescent substrate (ThermoFisher Scientific, USA). Chemiluminescence was measured with the Amersham Imager 600 (GE Healthcare, USA).

Confocal microscopy—Cells were stained with Hoechst (nucleus) and anti-CD45-AlexaFluor488 (cell membrane). Following, cells were resuspended in PBS and images were taken with a Zeiss confocal microscope. The images were created by merging 11 frames from a Z-stack with 0.3 μm per frame.

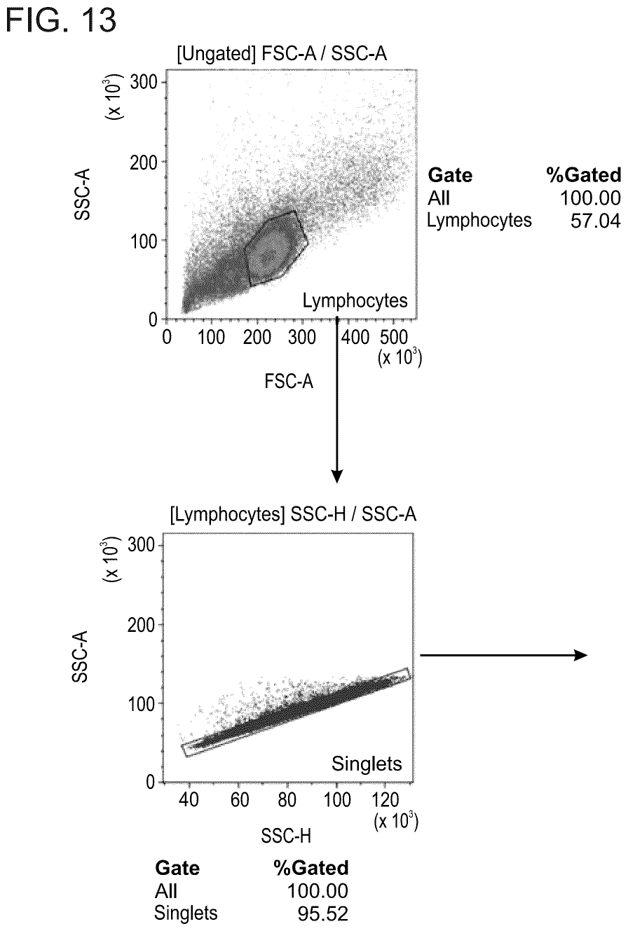

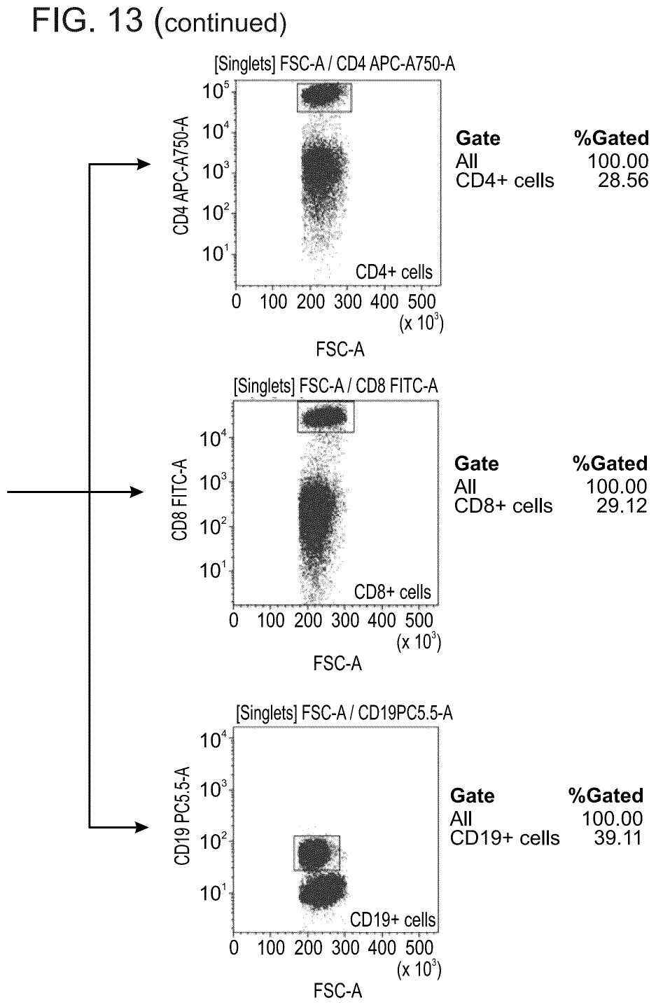

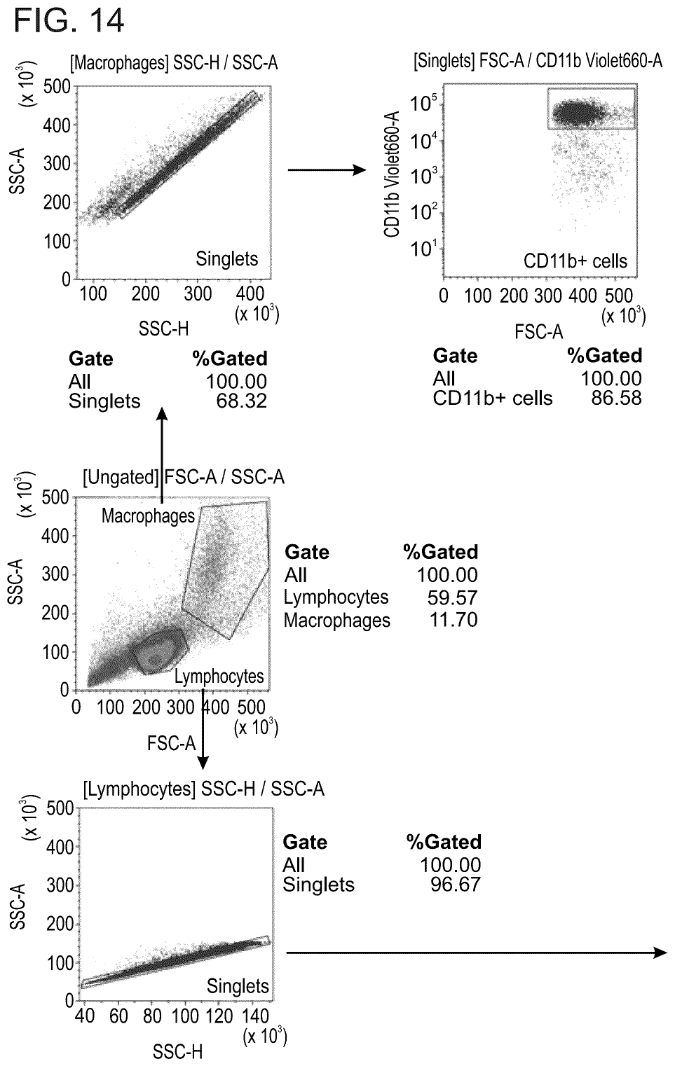

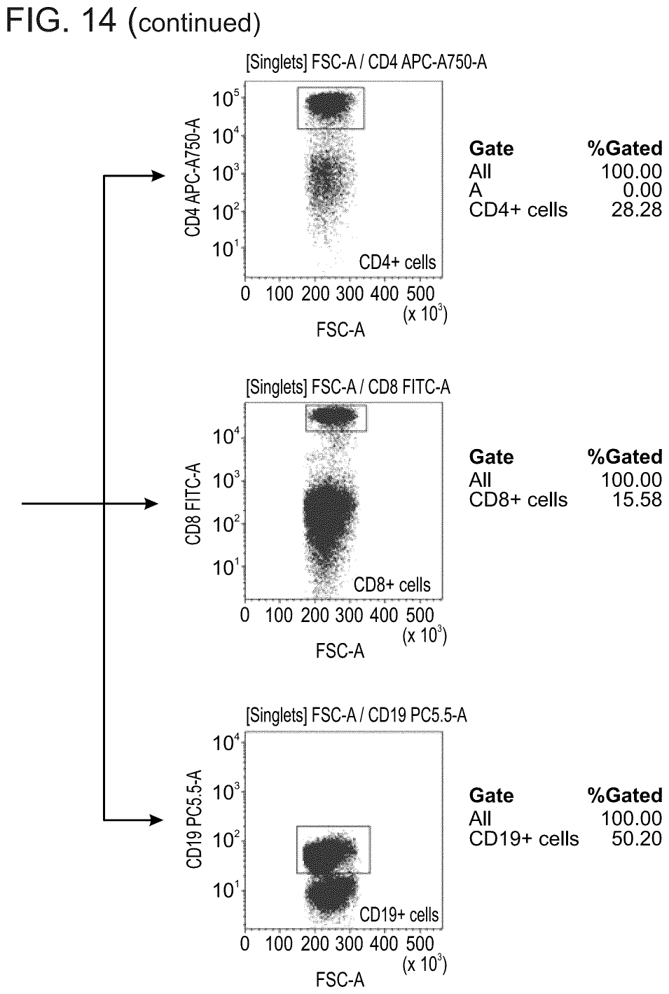

Ex-vivo binding—Leukocytes were extracted from the spleen and mesenteric lymph nodes (mLN) of both healthy C57B1/6 mice and IL-10 KO mice that spontaneously developed colitis. For the mLN, tissue was homogenized and cells were strained through a 70 μm cell strainer. Cells were washed with PBS, centrifuged and the pellet was washed again with PBS and resuspended as a single cell suspension. For the spleen, tissue was homogenized, cells were strained through a 70 μm cell strainer. Following a single wash with PBS, red blood cells were lysed with ddH₂O for a few seconds followed by addition of 10× HBSS to restore the solution to physiological conditions. Cells were strained a second time to ensure a single-cell suspension. Next, cells were stained with the appropriate cell surface markers (CD4, CD8, CD19, CD11b (Biolegend, USA, catalog numbers 100414, 100723, 115509 and 101259, respectively) and allowed to bind to either D1D2-LNPs, DATK32-LNPs or the respective control mD1D2-LNPs or isotype-LNPs. Following a 20-minutes incubation at 4° C., cells were washed and resuspended in FACS buffer. LNP binding for each leukocyte subpopulation was determined by the level of Cy5 as measured by flow cytometry. See FIG. 13 for the gating strategy used for cells from the mesenteric lymph nodes. See FIG. 14 for the gating strategy used for cells from the spleen.

Animal experiments—The Tel Aviv Institutional Animal Care and Use Committee approved the animal protocols for all in-vivo studies in accordance with current regulations and standards of the Israel Ministry of Health.

In-Vivo Gene Silencing:

Wild type C57BL/6 and IL-10 KO C57BL/6 mice were kept in a specific-pathogen-free animal facility at Tel Aviv University. For the PAC colitis model, piroxicam was administered to IL-10 KO mice in the chow at a concentration of 200 ppm for a total period of 11 days. Freshly prepared LNPs encapsulated with either siCD45 (SEQ ID NO: 20, 41) or siNC (SEQ ID NO: 18-19) were conjugated to RG7 and purified using CL4B resin, as described hereinabove. Note that, in a 5′ to 3′ direction, bases No. 1, 2, 5, 6, 10, 11, 12, 13 and 18 of SEQ ID NO: 20, bases No. 13 and 17 of SEQ ID No. 41, bases No. 1, 2, 4, 6, 12, 14, 16, 18, 24 and 25 of SEQ ID NO: 18 and bases No. 3, 9, 11, 13, 23, 25, 26 and 27 of SEQ ID NO: 19 are modified 2′-O-Methyl RNA bases which provide resistance against nucleases; nucleotides No. 20 and 21 of SEQ ID NO: 20 and nucleotides No. 20 and 21 of SEQ ID NO: 41 are deoxy-ribonucleotides (and not ribonucleotides) and are linked together with a phosphorothioate bond; and the last nucleotide of SEQ ID NO: 18 is also a deoxy-ribonucleotide (and not ribonucleotide).

The LNPs were injected 7 days following administration of piroxicam, and CD45 expression in various organs was assessed 4 days post-injection. For healthy mice, CD45 expression was assessed 4 days post-injection. After sacrificing the animals, organs were homogenized and single cell suspensions were obtained using 70 μm cell strainers (Corning, USA). Cells were stained with antibodies against CD3 (Brilliant Violet 421), CD4 (APC-Cy7), CD8a (AlexaFluor 488), CD19 (PE-Cy5.5), CD11b (Brilliant Violet 650), F4/80 (Brilliant Violet 605) and CD45 (AlexaFluor 647) and analyzed by flow cytometry. All antibodies were purchased from BioLegend, USA.

In-Vivo Safety Study:

10-weeks-old C57B1/6 female mice (Harlan laboratories) were injected with LNPs at a dose of 1.5 mg/kg and sacrificed 24 hours later. Blood was collected and analyzed by A.M.L. Israel for complete blood count (Sysmex and Advida-120) and biochemistry (Cobas-6000). Liver samples were used for histology (Histospeck, Israel). Splenic TNF-α and IL-6 levels were measured by DuoSet ELISA kits (R&D Systems, USA).

Therapeutic Efficacy Studies with siIFNγ:

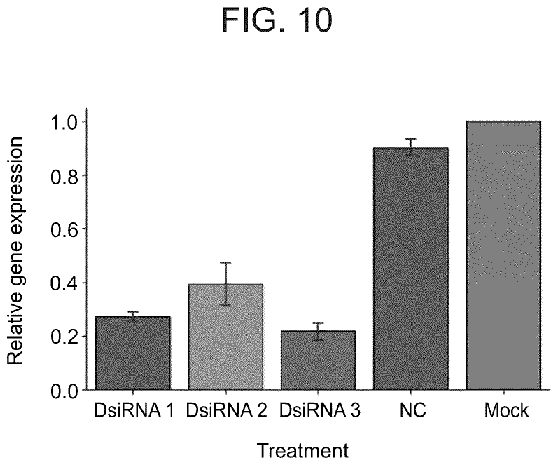

To test in-silico optimized siRNA sequences against murine IFNγ, HEK293 cells stably expressing murine IFNγ were generated by transfecting the cells with a pcDNA3 plasmid harboring the murine IFNγ gene. Stably expressing cells were selected with G418. Next, cells were transfected with each of the siRNA sequences and with a control sequence (siNC). 48 hours post-transfection, cells were lysed, RNA was extracted and cDNA was generated. Silencing efficiency was determined using qPCR with SYBR green (see FIG. 10). The sequence that most efficiently silenced IFNγ (SEQ ID NO: 9-10) was used for the subsequent efficacy experiments. Note that, in a 5′ to 3′ direction, bases No. 1, 2, 4, 6, 12, 14, 16, 18 and 24 of SEQ ID NO: 9 and bases No. 3, 9, 11, 13, 23, 26 and 27 of SEQ ID NO: 10 are modified 2′-O-Methyl RNA bases which provide resistance against nucleases; and nucleotide No. 25 of SEQ ID NO: 9 is a deoxy-ribonucleotide (and not ribonucleotide).

For the efficacy studies, colitis was induced in 9-weeks-old female C57BL/6 IL-10 KO mice by mixing piroxicam (200 ppm) in the food. Freshly prepared LNPs encapsulated with either siIFNγ (SEQ ID NO: 9-10) or siNC (SEQ ID NO: 18-19) were conjugated to RG7 and purified using CL4B resin, as described hereinabove. LNPs were injected intravenously at day 4, 6, 8 and 10, post piroxicam administration. The volume of administered LNPs was calculated with the Ribogreen assay (Thermo Fisher Scientific, USA) for each LNP preparation to ensure a consistent dose of 1.5 mg siRNA per kg body weight. 30 minutes prior to injection, LNPs were mixed with the D1D2-Fc or mD1D2-Fc (final protein concentration of 60 μg/ml) and the total volume was completed to 200 μl. As a positive control, a monoclonal antibody against TNF-α was used (Clone MP6-XT22) that blocks the pro-inflammatory cytokine TNF-α.

Animals were randomized before piroxicam treatment and the study was performed in a double-blinded fashion. The experiment was performed by a CRO to ensure that the investigator was blinded during group allocation.

Body weight was recorded daily and mice were sacrificed at day 11 of the experiment. The colon was harvested to measure the length, to analyze colonic cytokine levels and to perform colon histology. Blood samples were collected to measure IL-6 and IL-1β expression levels.

Molecular imaging studies—1,4,7-Triazacyclononane-1,4-bis-acetic acid-7-maleimidoethylacetamide (NOTA-mal: Macro cyclics; Dallas, TX) was conjugated to MAdCAM-D1D2-Fc or MAdCAM-mD1D2-Fc fusion protein using a previously described approach with minor modifications³². Briefly, to 500 μg of fusion protein in 150 μl of phosphate buffer (pH 7.0) was added to freshly prepared 2-iminothiolane and then NOTA-mal in phosphate buffer (10% dimethyl sulphoxide) and 2IT such that final concentration ratios were: fusion protein, 1:NOTA-mal, 20:2IT, 10. The reaction was mixed by gentle pipetting, briefly centrifuged, and then placed in a 37° C. water bath for 30 minutes. Unbound chelator was removed using centrifugal filter units (3 kDa MW cut-off; Centricon, Millipore, Billerica MA), and the immunoconjugate was concentrated into phosphate buffer (0.1 M, pH 7.0) and stored in aliquots at −80° C.

For the radiolabeling, 6 volume equivalents of sodium acetate buffer were added to 178 GBq (4.78 mCi) ⁶⁴Cu in 5 μL HCl (0.04 N). The MAdCAM-D1D2-Fc or MAdCAM-mD1D2-Fc fusion protein (177 μg in 61 μL phosphate buffer) was then added to 15.8 μl of ⁶⁴Cu solution (31.7 MBq (858 μCi)). Following a 30 minutes incubation at room temperature, the degree of radiolabeling was assessed by thin-layer chromatography (TLC; Whatman No. 1 paper eluted with phosphate buffer; 0.1 M, pH 8, 100 mM EDTA) and radiochemical purity was found to be >95%. The radioimmunoconjugate was diluted with saline and sterile filtered (0.2 μm) before injection.

For the imaging studies, ⁶⁴Cu-labeled MAdCAM-D1D2-Fc or MAdCAM-mD1D2-Fc fusion protein (22 μg antibody, 3.12 MBq (84.3 μCi)) was injected into the tail vein. The mice were then anesthetized using isoflurane (1-4% in oxygen) and placed in a Bruker Albira multimodality (PET/CT) small-animal imaging system (Bruker Corporation; Woodbridge CT) for imaging. PET/CT data were collected for 30 minutes at 1, 3 and 24 hours post-injection (p.i.). At 24 hours p.i., the mice were euthanized by CO₂ inhalation, and an ex-vivo biodistribution analysis was carried out. Tissues were collected and weighed, and the radioactivity was assayed. The large colon was excised, measured and weighed to confirm the presence of colitis in the piroxicam treated group. PET and CT images were registered manually using AMIDE software³³. Data from volumes of interest (VOIs) were used to calculate the biodistribution in selected tissues for the small animal PET imaging studies.

Statistical analysis—Data in the bar charts is expressed as mean ±95% confidence interval (CI). Boxplots center line represents the median, box represents the interquartile range and whiskers represent minimum and maximum values. Statistical analysis was performed in Python and GraphPad Prism. In general, when comparing 2 groups (for instance mD1D2 to D1D2), Student's t-test was used, and a one-way ANOVA was used when comparing 3 or more groups. A more complex statistical model (2-way interaction mixed model) was used in the molecular imaging section to confirm the correlation between colitis severity and D1D2 uptake. More specifically, for the CD45 silencing, a two-sided student's t-test was performed between the mD1D2-LNPs and D1D2-LNPs groups. For the toxicity study, a one-way ANOVA test was performed to exclude significant differences between any of the groups. For the efficacy study, a one-way ANOVA with Dunnett's post hoc test was used to demonstrate a significant difference between the D1D2-IFNγ-LNPs group and the negative control groups (D1D2-siNC-LNPs, mD1D2-IFNγ-LNPs and mock-treated). The same post hoc test revealed a significant difference between the groups D1D2-IFNγ-LNPs and mAb TNF-α only in the colonic TNF-α expression levels (p<0.05). Group size for the in-vivo silencing and toxicity study was 5 mice per group. In the efficacy study, group size was 12 mice per group. In all figures * for p<0.05, ** for p<0.01 and *** for p<0.0001.

### Example 1

### Generation of a Novel MAdCAM-1-Fc Fusion Protein

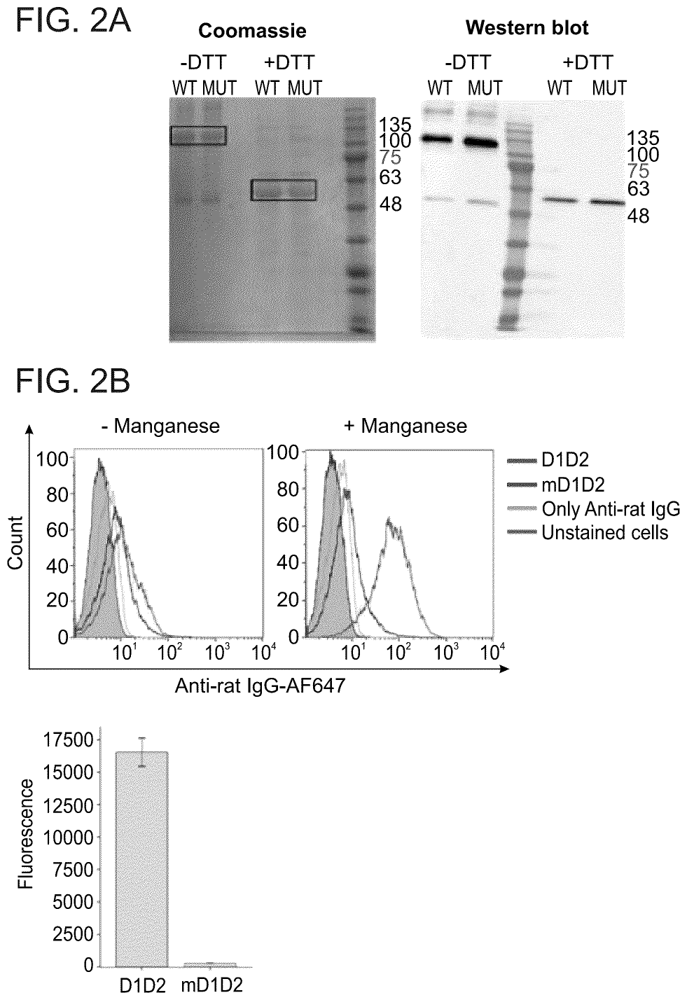

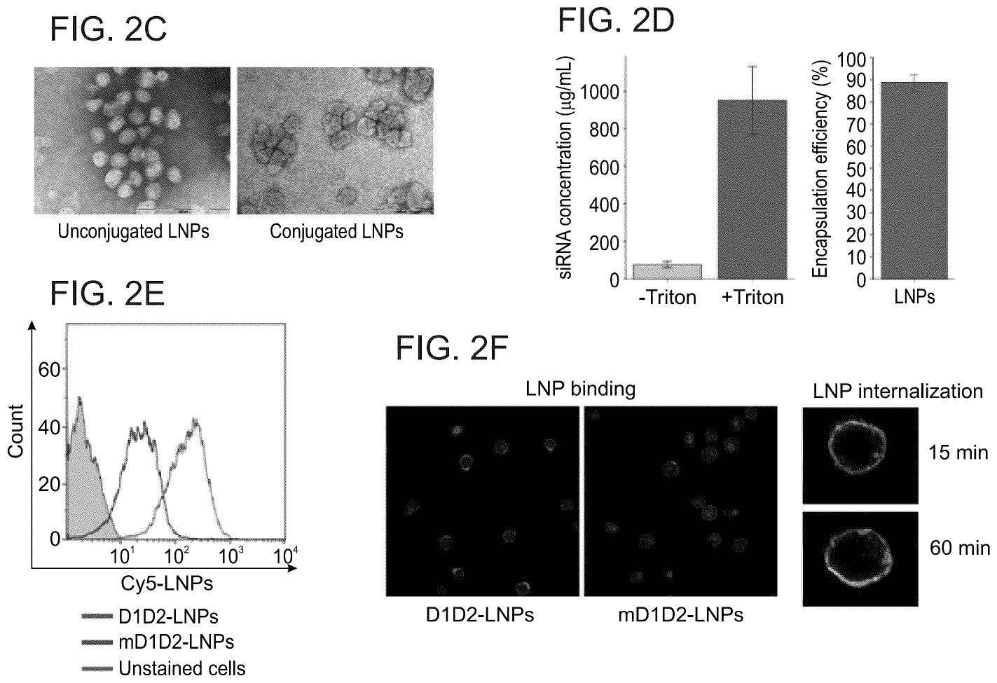

The present inventors have designed a recombinant fusion protein containing two domains of the intestinal endothelium ligand MAdCAM-1, namely the integrin binding domains D1 and D2, fused to an Fc domain, hereinafter D1D2-Fc. Specifically, domains D1 (SEQ ID NO: 11) and D2 (SEQ ID NO: 12) of murine MAdCAM-1 were fused to the N-terminus of the Fc domain of rat IgG₂ₐ (including the hinge, excluding C_{H}1, SEQ ID NO: 13). In addition, a signal peptide for secretion and a FLAG-tag were added to the N-terminus of the construct and a 6× HIS-tag was added at the C-terminus for purification purposes (see FIGS. 1A-C and 11-12, SEQ ID NOs: 14-15). Purity and size (˜50 kDa) were confirmed by SDS-PAGE (FIG. 2A).

A mutated version of the D1D2-Fc was also generated, hereinafter mD1D2-Fc, and served as a negative control. Specifically, the mutated fusion comprised a D62A mutation corresponding to SEQ ID NO: 25 (see FIGS. 11-12, SEQ ID NOs: 16-17); this mutation in the D1 domain, has been reported to severely affect the ability of MAdCAM-1 to bind α₄β₇ integrin^{14,15}.

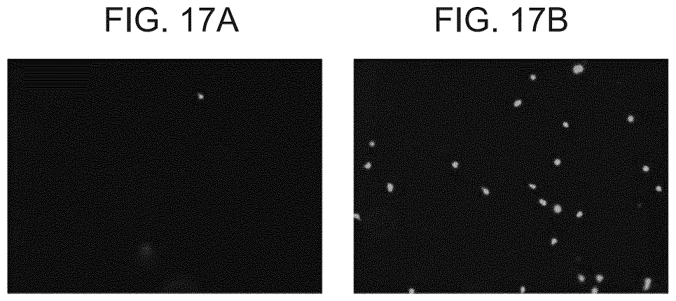

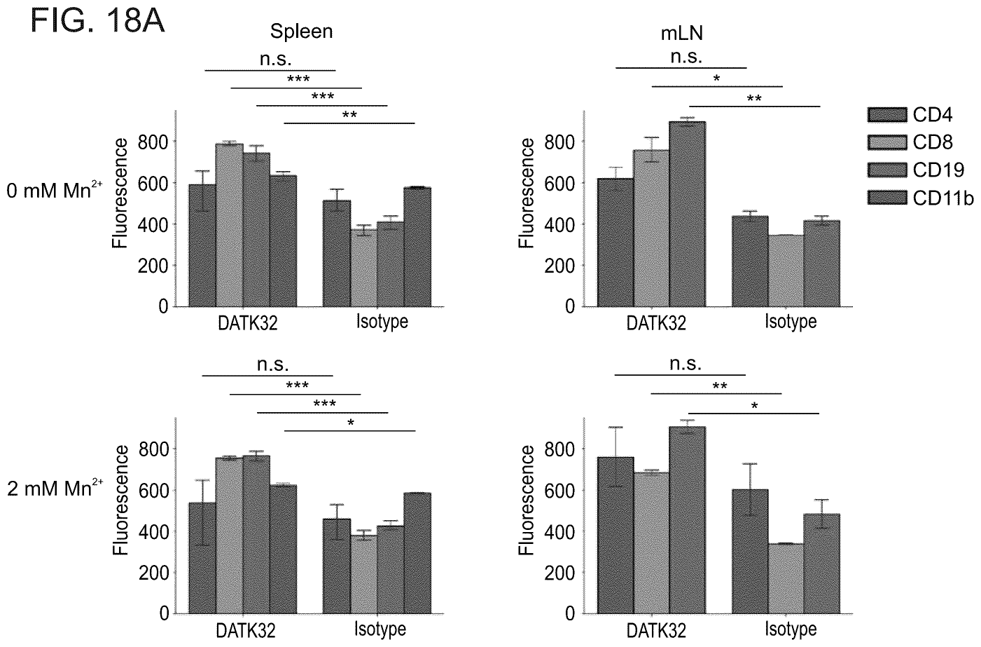

Following purification, the functionality of the generated D1D2-Fc and mD1D2-Fc was tested in-vitro by assessing their binding to TK-1 cells using flow cytometry. TK-1 cells have been shown to express high levels of α₄β₇ integrin^{10,16} and are therefore an excellent in-vitro model to test the functionality of the recombinant MAdCAM-1 constructs. Cells were either treated with Mn²⁺ to create the high affinity (HA)-α₄β₇ or with Ca²⁺ as a low affinity (LA)-α₄β₇ control. To verify this specificity, exclusive adhesion was confirmed in the presence of Mn²⁺ to a monolayer of HEK293 cells that were stably transfected with membrane-bound MAdCAM-1 (FIGS. 17A-B). As expected, in the absence of Mn²⁺, no significant cell binding was visible with both D1D2-Fc and mD1D2-Fc. However, following Mn²⁺ treatment, while the mD1D2-Fc did not bind the cells, the D1D2-Fc significantly bound the activated cells expressing the HA-α₄β₇ (FIG. 2B). This demonstrated that the generated D1D2-Fc is functional and can distinguish between the HA and LA α₄β₇ conformation; and that the mD1D2-Fc is a suitable negative control.

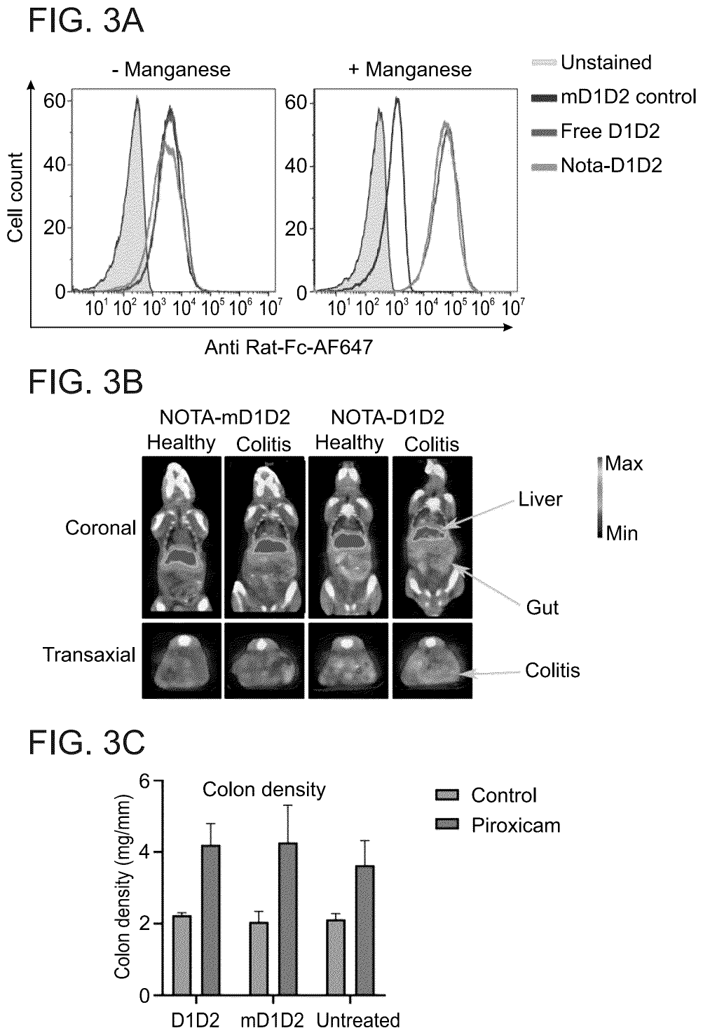

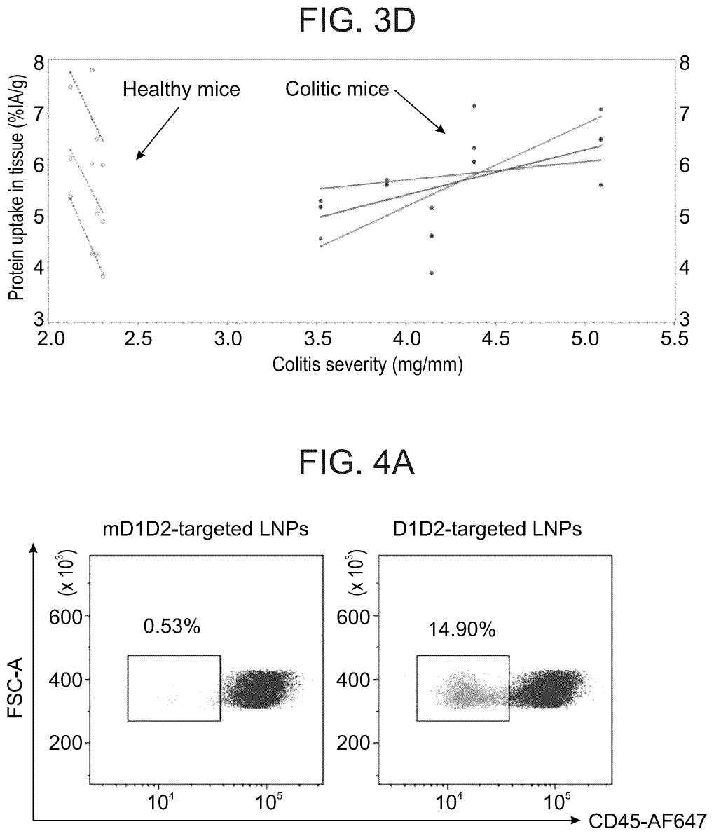

After validating that the D1D2-Fc protein binds exclusively to the HA conformation of integrin α₄β₇, the present inventors evaluated its ability to facilitate specific imaging of inflammatory leukocytes. To this end, each of the generated D1D2-Fc and mD1D2-Fc proteins was conjugated directly to a chelator, NOTA, to enable labeling with the radioisotope ⁶⁴Cu. Notably, the conjugation of the D1D2-Fc to NOTA did not affect protein functionality, as was tested by binding to TK-1 cells in vitro with flow cytometry (FIG. 3A). Following, the protein-NOTA-⁶⁴Cu conjugate was injected to healthy mice and to mice with colitis and 24 hours post-injection the mice were imaged by PET/CT. Injection of the radiolabeled D1D2 protein did not affect the colitis severity (FIG. 3C). As shown in FIG. 3B, injection of the D1D2-NOTA-⁶⁴Cu resulted in an increased uptake in the gut of colitic mice while the signal in the liver decreased. The transaxial image in particular clearly displays the preferential gut uptake of the D1D2-NOTA-⁶⁴Cu in mice with colitis. The difference in gut uptake of the mD1D2-NOTA-⁶⁴Cu conjugate between healthy and colitic mice was not as dramatic. This uptake was significantly correlated to colitis severity with n=5 mice/group (p=0.014). A detailed statistical analysis on the correlation of colitis severity and specific D1D2-Fc uptake is shown in FIG. 3D.

### Example 2

### Generation of Lipid Nanoparticles Comprising a MAdCAM-1-Fc Fusion Protein as a Targeting Moiety

In the next step the generated D1D2-Fc fusion protein was used to decorate the surface of lipid nanoparticles (LNPs) in order to produce targeted LNPs with the ability to deliver e.g. siRNA specifically to these leukocytes.

To generate uniformly-sized LNPs that have a high siRNA encapsulation efficiency and minimal batch-to-batch variation, the NanoAssemblr™ microfluidic mixing device¹⁹; and an LNP formulation that was previously reported^{4,5,21} using the ionizable lipid, DLin-MC3-DMA, which aids in siRNA encapsulation²⁰, were utilized. The generated LNPs had mean diameter of ˜40 nm and a zeta potential of ˜−10 mV (Table 2 hereinbelow) and siRNA encapsulation efficiencies were close to 90% (FIG. 2D) with polydispersity index of less than 0.2 (Table 2 hereinbelow). The size and uniformity of the particles was confirmed using transmission electron microscopy (FIG. 2C).

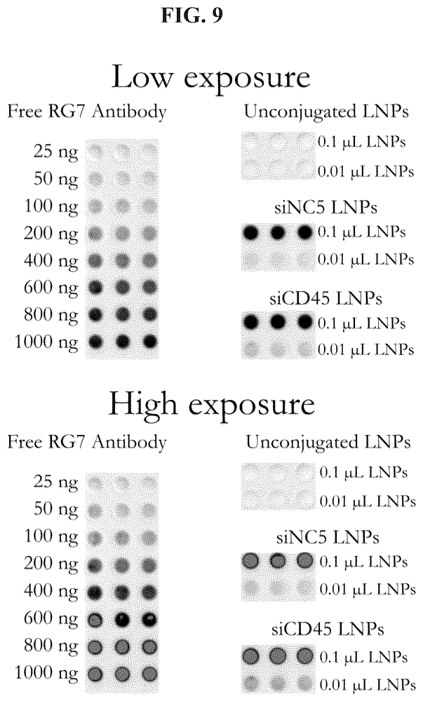

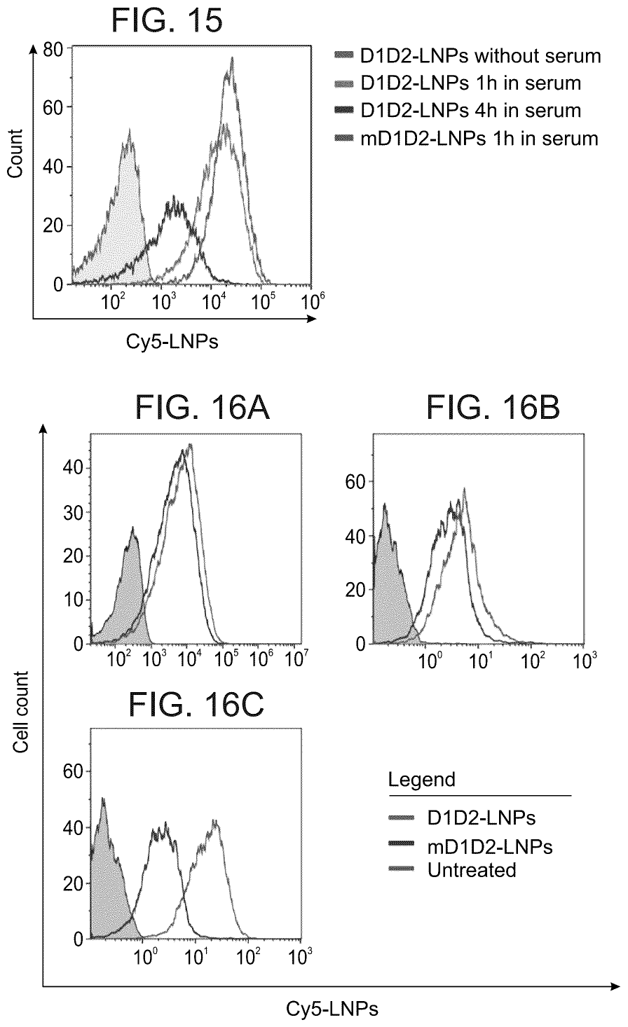

Next, the LNPs were conjugated to RG7, a monoclonal antibody against rat IgG₂ₐ, using maleimide-thiol chemistry. The presence of RG7 on the surface of the LNPs was confirmed by dot blot (FIG. 9). In this way the targeting protein (e.g. D1D2-Fc) can be bound to the LNP surface through affinity by the RG7 linker. Thus, the D1D2-Fc or mD1D2-Fc were subsequently attached to the RG7-LNPs by adding the protein to the LNPs solution to generate D1D2-targeted and mD1D2-targeted LNPs. The optimal concentration of the targeting protein was optimized such that the minimal amount of protein that achieves the highest level of LNP binding to TK-1 cells was added. Further, this linkage was sufficiently stable in freshly isolated mouse blood plasma for at least 1 hour at 37° C., making this approach feasible for in vivo administration (FIG. 15).

In addition, the RG7 conjugation strategy was compared with two other options: direct conjugation to the DSPE-PEG-maleimide lipid using reduced cysteine residues in the D1D2 protein or by using a previously published conjugation strategy that involves ASSET (Anchored Secondary ScFv Enabling Targeting), a lipidated scFv against rat IgG₂ₐ that readily incorporates in the LNPs [Kedmi, R. et al. Nat. Nanotechnol. 13, 214-219 (2018)]. When ASSET is incorporated into the LNPs, it can bind the D1D2-Fc by affinity. As shown in FIGS. 16A-C, the RG7-mediated conjugation was superior over the other two methods.

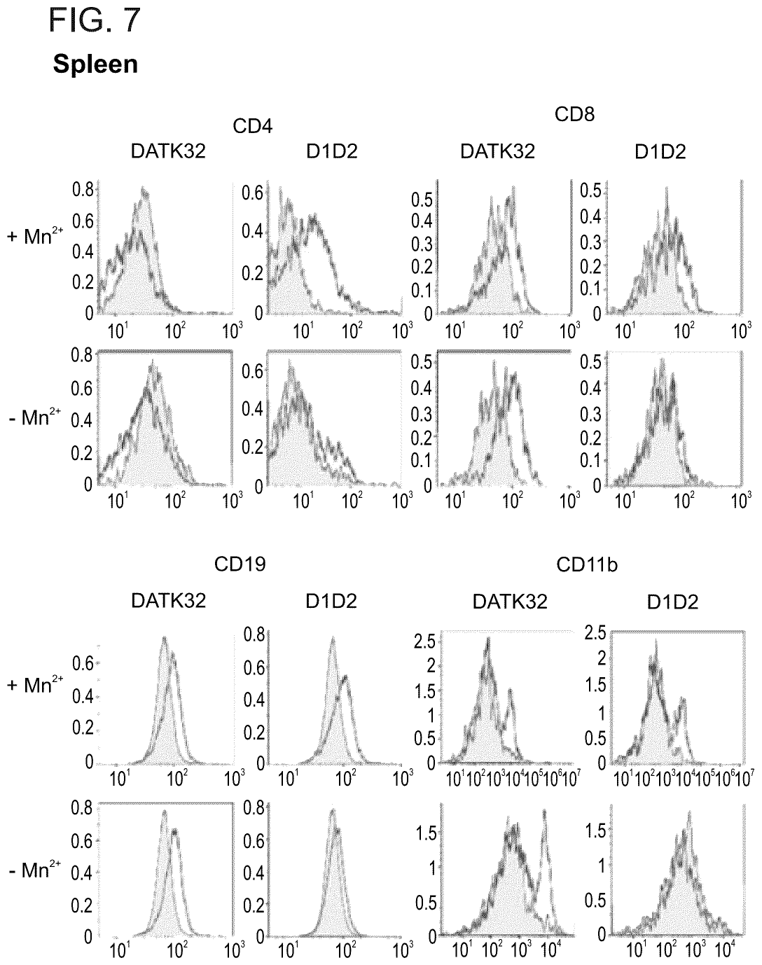

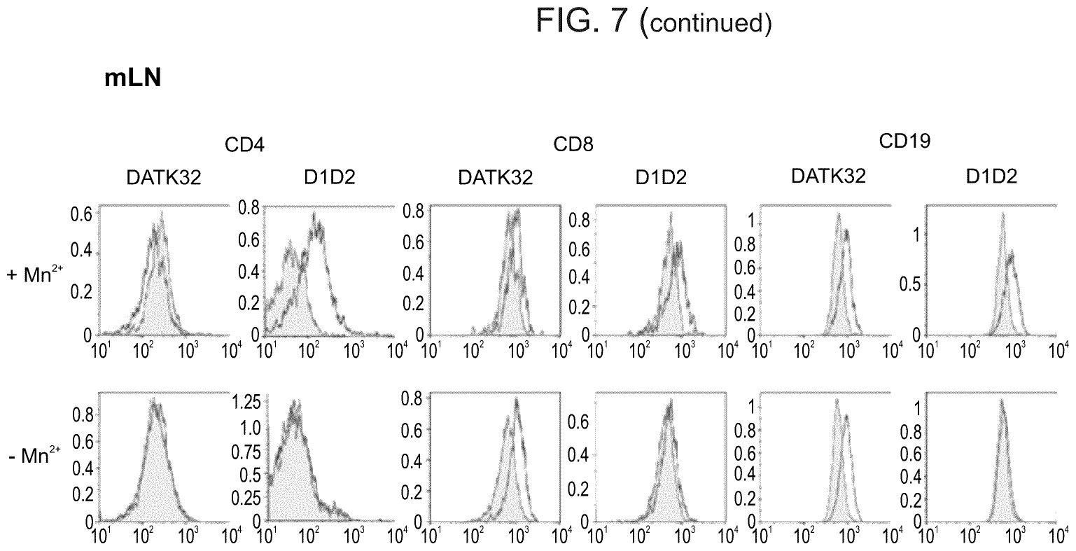

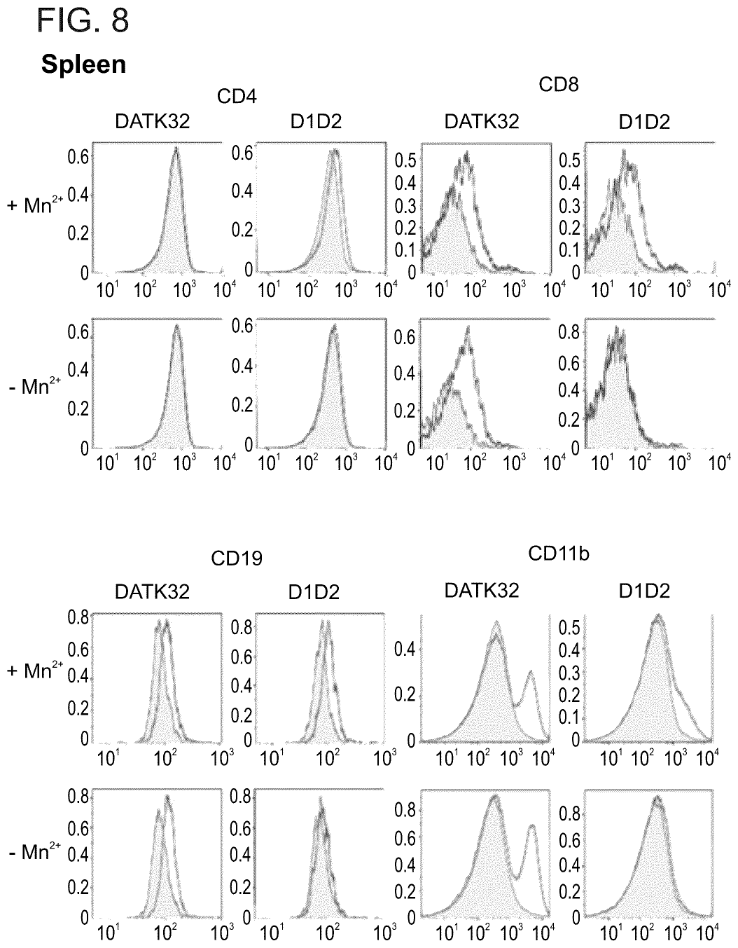

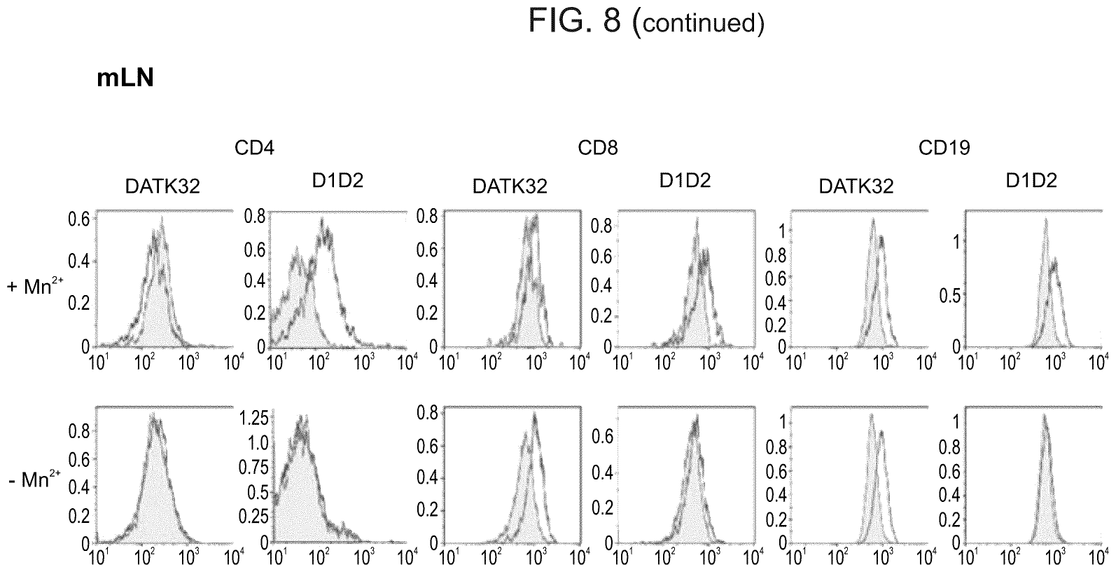

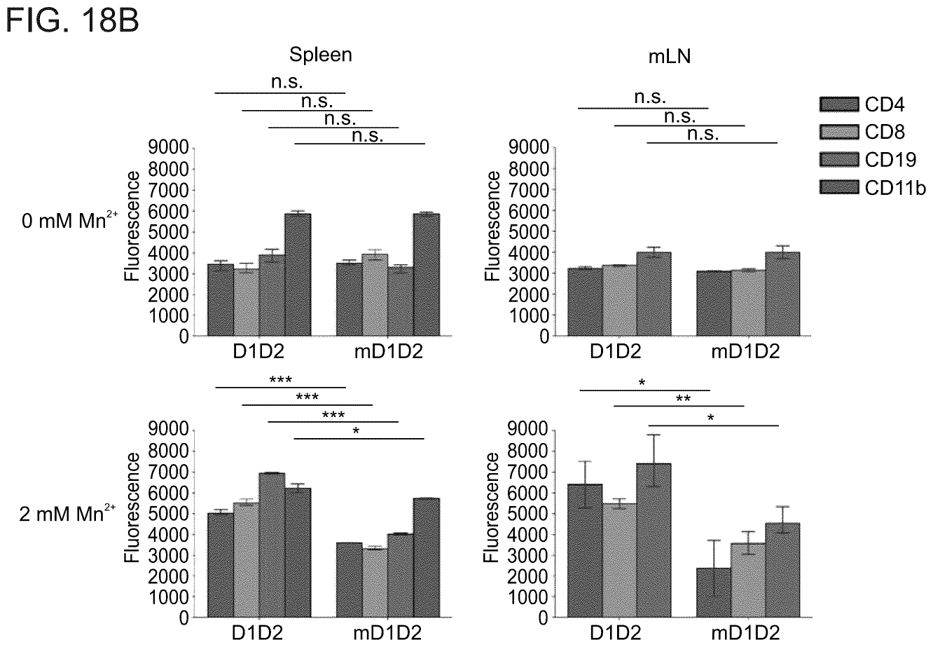

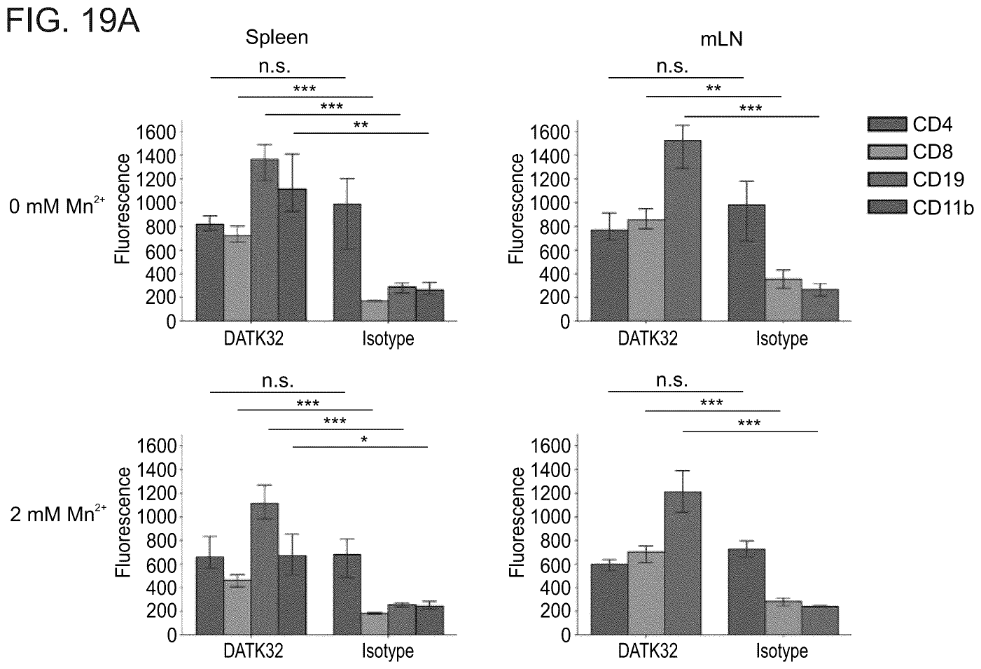

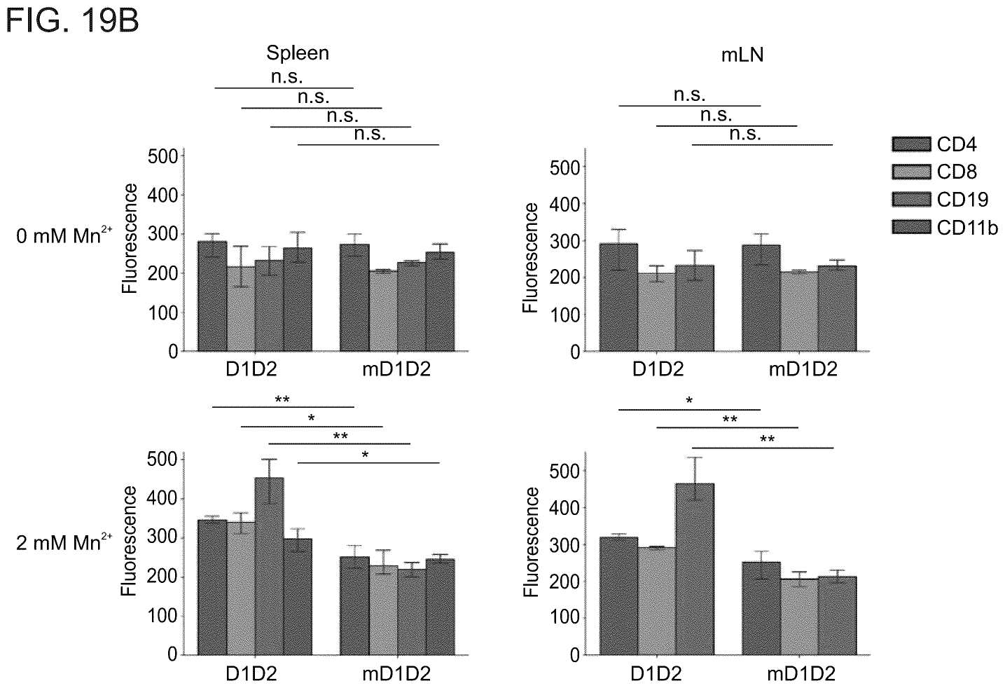

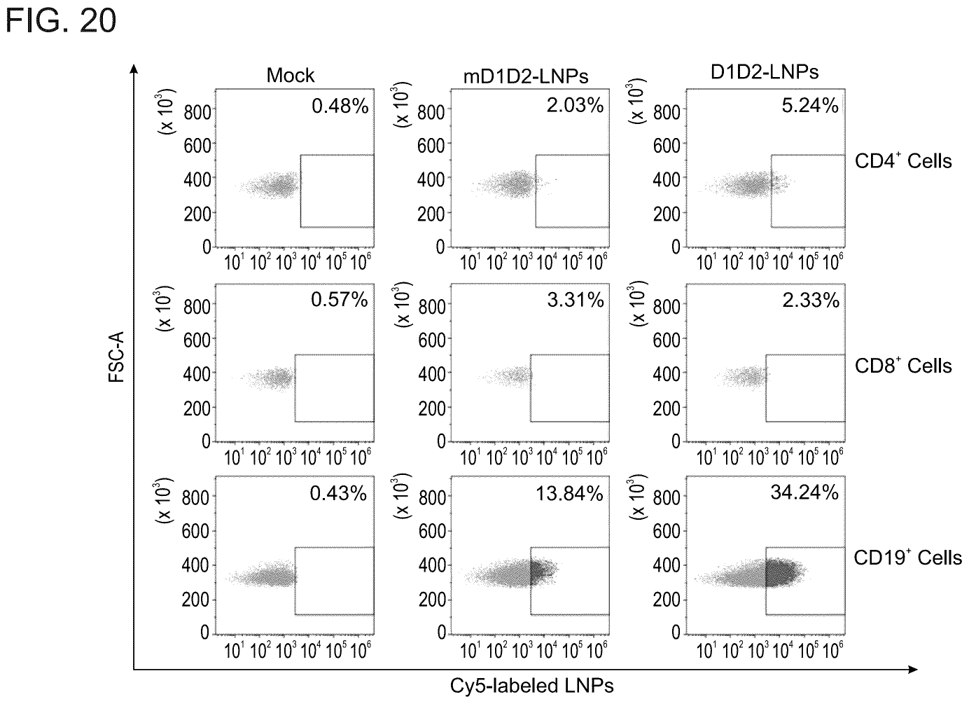

Following preparation, conjugation and purification of the LNPs, the functionality of the D1D2-targeted LNPs was tested. To this end, binding of targeted LNPs encapsulating Cy5-siRNA (SEQ ID NO: 9-10) to cells (Mn²⁺ or Ca²⁺ treated) was tested by flow cytometry. As shown in FIG. 2E, LNPs' binding increased dramatically when targeted by D1D2 as compared to mD1D2. Furthermore, confocal microscopy indicated internalization of the Cy5-labeled, D1D2-targeted LNPs (FIG. 2F). Next, primary leukocytes from spleen and mesenteric lymph nodes (mLN) were extracted from healthy mice or mice with colitis and binding of D1D2-targeted LNPs was compared to both mD1D2-targeted and DATK32-targeted (a conformational insensitive mAb against α₄β₇ integrin) LNPs. As expected, the DATK32 antibody-targeted LNPs were unable to distinguish between HA and LA α₄β₇ integrin (as it displays similar binding levels between the Mn²⁺-treated group and the control group). Surprisingly, the DATK32-targeted LNPs were unable to bind to CD4+ T-cells. D1D2-targeted LNPs, on the other hand, demonstrated a strong binding preference to Mn²⁺-activated cells (see FIGS. 7-8 and 18A-19B). Notably, D1D2-targeted LNPs strongly bound to CD4⁺ T-cells in a conformation-dependent manner. When injected intravenously, the LNPs were also able to bind primary leukocytes. Splenocytes were extracted 1 hour post-injection and Cy5-labeled cells were identified by flow cytometry (FIG. 20).

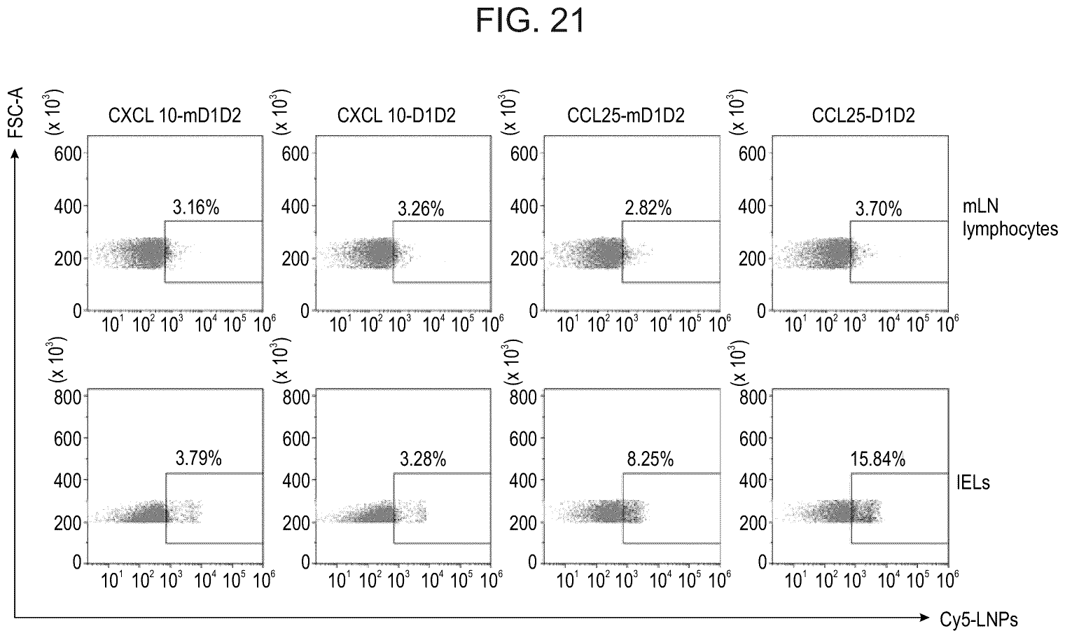

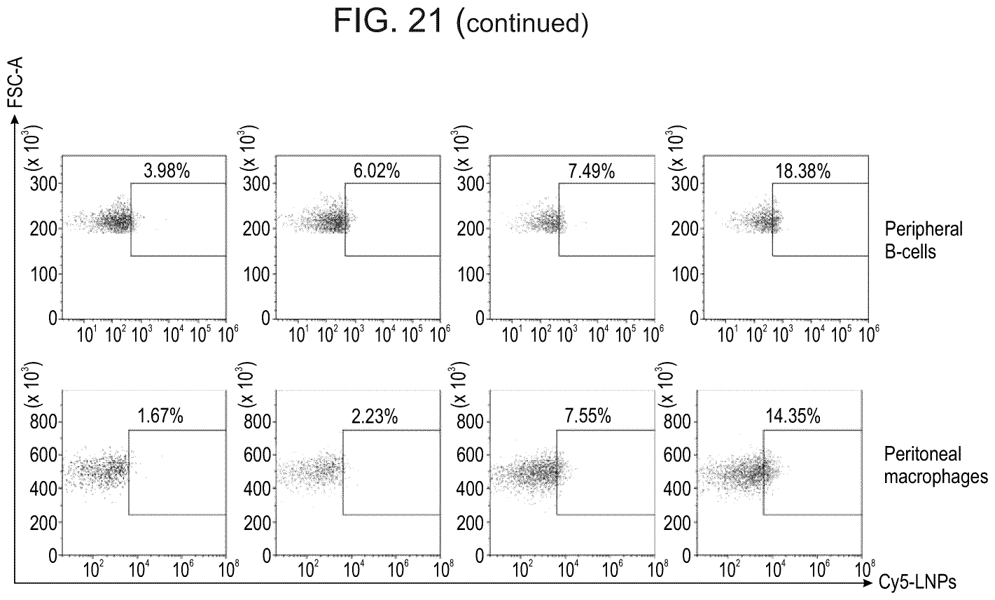

CCL25 specifically increases α₄β₇ integrin's affinity for MAdCAM-1 by binding to the CCR9 receptor. As expected, CCL25 treatment of cells enhances D1D2-targeted LNP binding compared to CXCL10-treated control cells (CXCL10 increases α₄β₇ integrin's affinity for VCAM-1), see FIG. 21.

TABLE 2
Hydrodynamic size, polydispersity index (PDI)
and zeta potential of the produced LNPs.

| | Hydrodynamic | | Zeta potential |
|---|---|---|---|
| LNPs | size (nm) | PD1 | (mV) |
| Unconjugated LNPs | 40.61 ± 2.60 | 0.13 ± 0.01 | −10.91 ± 1.44 |
| RG7-LNPs | 99.71 ± 4.75 | 0.18 ± 0.01 | −6.78 ± 1.47 |

### Example 3

### MAdCAM-1-Fc Fusion Protein Target Specific Delivery of a Payload to Gut-Homing Leukocytes in a Colitis Mouse Model

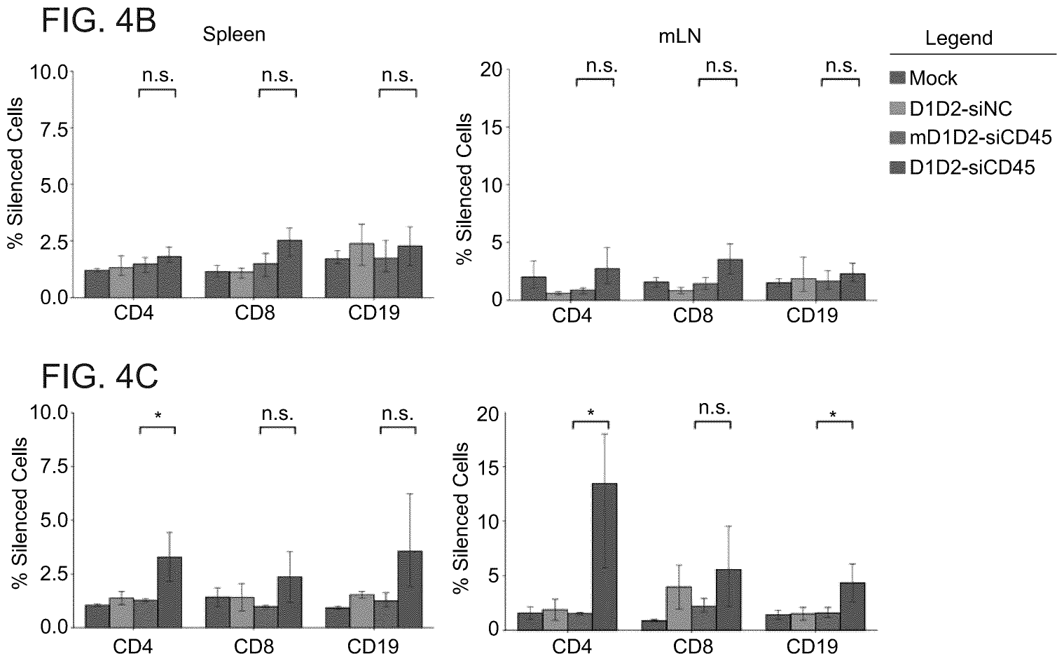

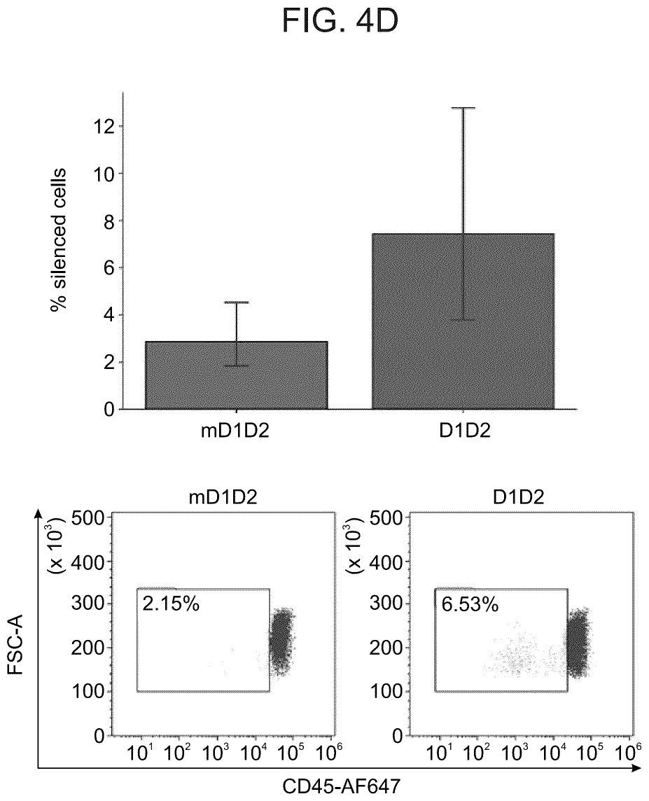

The feasibility of in-vivo gene silencing using the generated D1-D2-targeted LNPs was evaluated. In the first step, the strategy was used to silence a surrogate marker, the pan-leukocyte gene CD45. A piroxicam-accelerated colitis (PAC) in IL-10 knock-out (KO) mice on a C57BL/6 background^{23,24} was used as a model of experimental colitis. Of note, IL-10KO mice with active colitis have strongly increased levels of MAdCAM-1 in the gut (Connor, E. M., et al. doi:10.1002/j1b.65.3.349), pointing to the importance of MAdCAM-1 in IL-10 deficient mice. CD45 silencing in healthy or colitic mock-treated mice was compared to healthy or colitic mice, respectively, that were injected with either of three different LNP formulations: D1D2-LNPs loaded with negative control siRNA (siNC, SEQ ID NO: 18-19); D1D2-LNPs loaded with CD45 siRNA (siCD45, SEQ ID NO: 20, 41) and mD1D2-LNPs loaded with siCD45 (SEQ ID NO: 20, 41). Five days post intravenous injection of the targeted LNPs, cells from the mesenteric lymph nodes (mLNs) were collected as T-cells there are activated by gut-tropic APCs. Cells from the spleen were also collected as a control peripheral lymphoid organ. Cells were analyzed for their CD45 expression using flow cytometry (see FIGS. 13-14 for the gating strategy). As shown in FIG. 4C, following injection of siCD45 D1D2-targeted LNPs, silencing levels in the spleen were lower compared to the CD4 population in the mLN. In the mLNs, silencing levels in CD8⁺ T-cells and B-cells were lower compared to the CD4⁺ T-cell population. The CD4⁺ population was most effectively silenced by the siCD45 D1D2-targeted LNPs. As a negative control, healthy mice were also injected with D1D2-targeted LNPs encapsulating siCD45 yielding no significant results in any cell type from any organ (FIG. 4B). This result could be attributed to the fact that in healthy mice there are less circulating lymphocytes with α₄β₇ integrin configured in the HA conformation. Silencing results in colonic CD4⁺ T-cells were not statistically significant, probably due to the fact that these cells have the integrin reconfigured to the LA state as they use α_{E}β₇ for their retention (FIG. 4D).

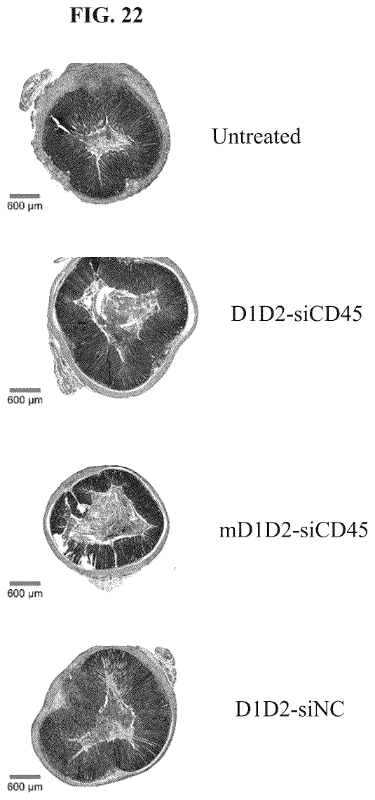

Injection of the different LNPs did not affect the colitis severity in the colon as was concluded from colon histology in mice from the different groups (FIG. 22).

Following, the potential adverse effects of the targeted LNPs was investigated. The presence of liver damage or systemic immune activation upon intravenous injection of the LNPs would provide a barrier for future clinical translation and hence render therapeutic efficacy studies obsolete. Thus, a toxicity study was performed to ensure that D1D2-targeted LNPs are safe upon systemic administration. Specifically, the levels of common liver enzymes secreted in the blood were measured and potential damage to the liver was assessed by histology. Furthermore, the immunogenicity of the targeted LNPs was assessed by performing a whole blood cell count and by measuring the levels of the pro-inflammatory cytokines TNF-α and IL-6 in the spleen. Two therapeutically irrelevant siRNAs were encapsulated to avoid interference with the immune system, namely the siCD45 (SEQ ID NO: 20, 41) and the siNC (SEQ ID NO: 18-19). The toxicity parameters in healthy mock-treated mice were compared to healthy mice that were injected with either of three different LNP formulations: D1D2-LNPs loaded with siNC; D1D2-LNPs loaded with siCD45 and mD1D2-LNPs loaded with siCD45.

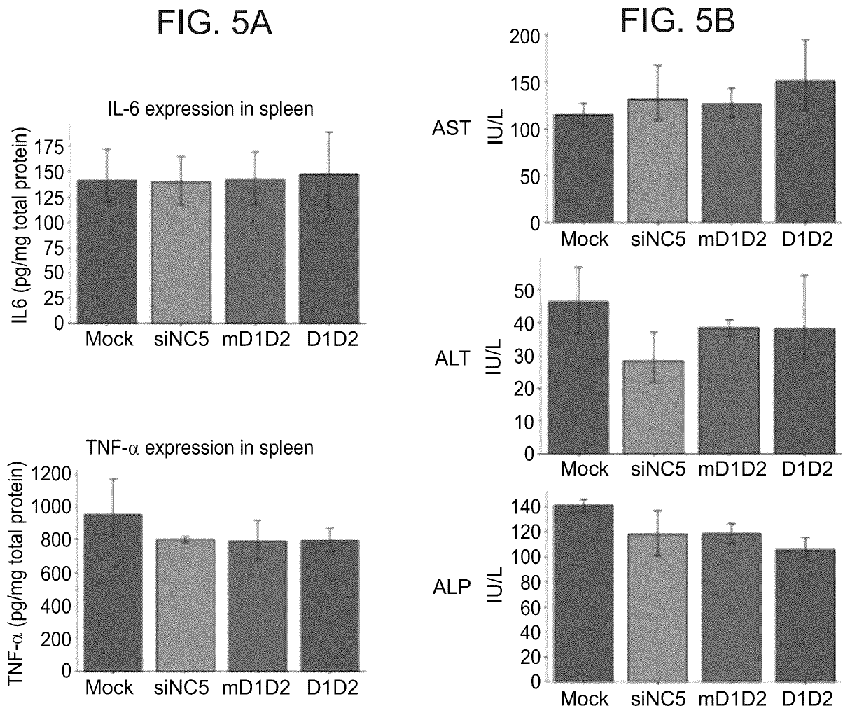

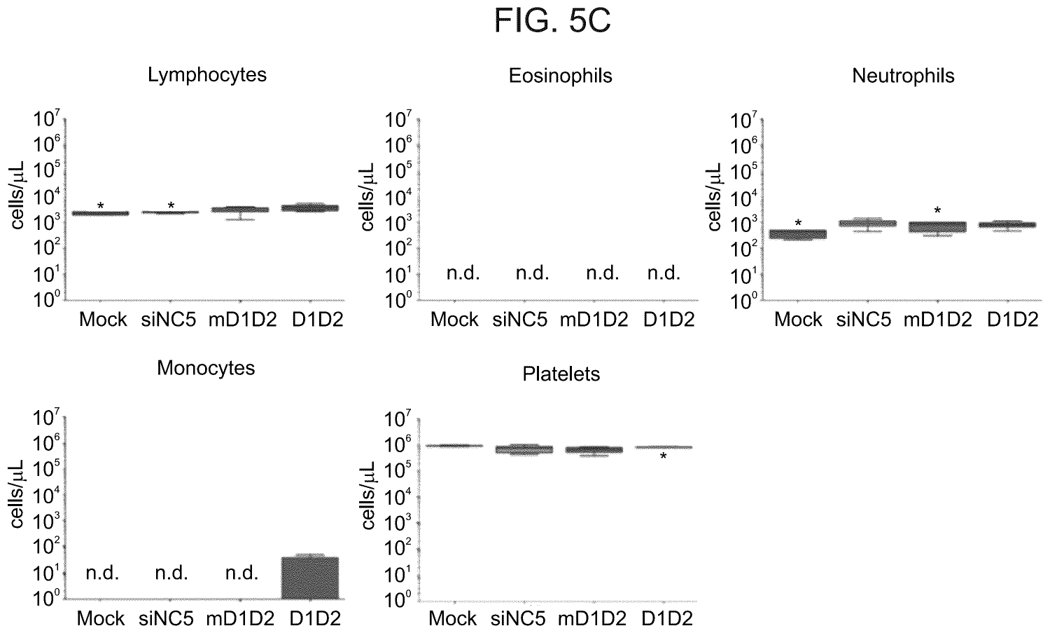

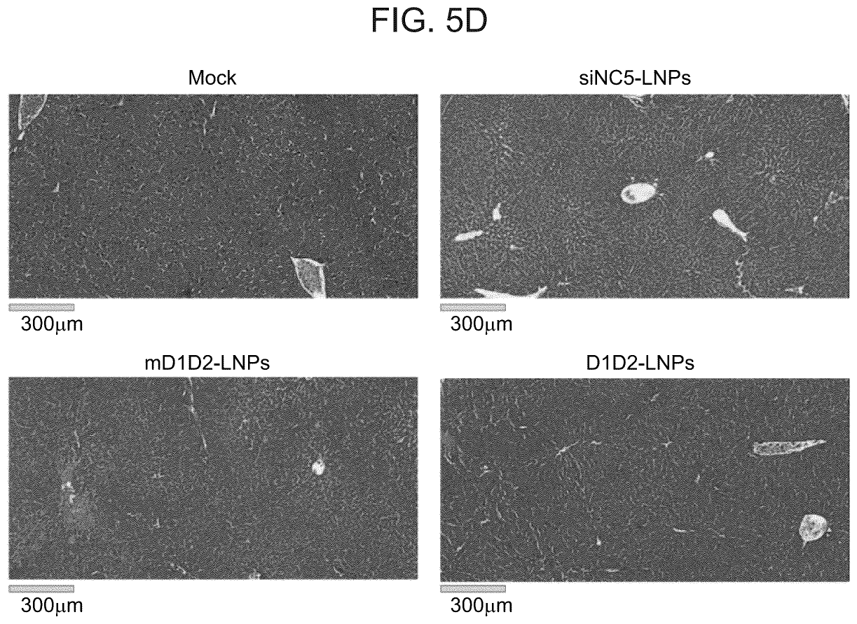

Upon injection of any of the LNP formulations, no elevation of the liver enzymes aspartate aminotransferase (AST), alanine transaminase (ALT) and alkaline phosphatase (ALP) was observed (FIG. 5B). In addition, liver histology did not reveal any excessive bleeding or liver damage (FIG. 5D). Thus, the injection of the LNPs did not cause any observable liver toxicity. Furthermore, none of the LNP formulations induced unwanted immune responses as measured by a change in blood count (FIG. 5C) and splenic TNF-α and IL-6 expression levels (FIG. 5A). There was no significant difference in counts of platelets, neutrophils and lymphocytes between the groups and counts of eosinophils and monocytes in the blood were undetectable in almost all groups.

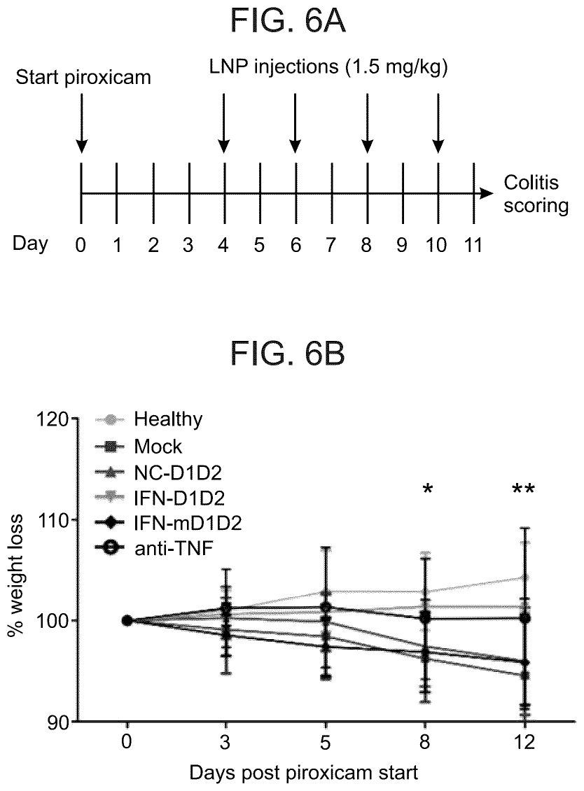

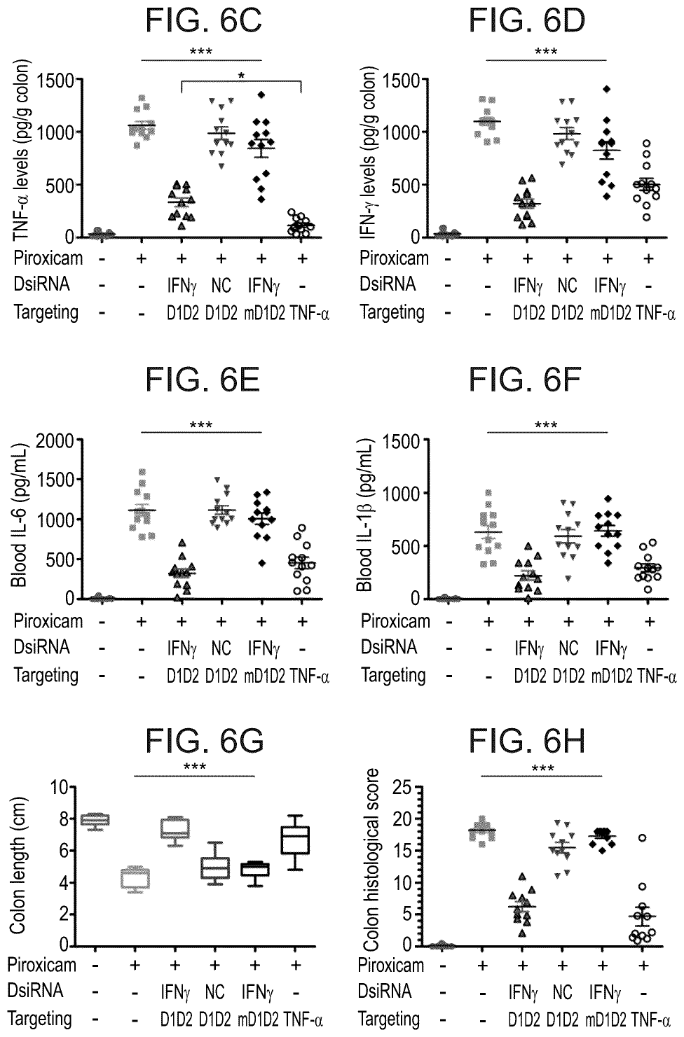

Based on the CD45 silencing results, a therapeutic target gene related to CD4⁺ T-cell biology was explored for therapeutic efficacy studies. Because IFNγ is secreted by inflammatory Th1 cells and as IFNγ is causatively involved in experimental colitis²⁵, this gene was chosen as a therapeutic target gene for an efficacy study using the generated D1D2-Fc as a targeting moiety in PAC IL-10KO mice. An optimized IFNγ siRNA (siIFNγ) sequence was selected and tested in vitro and resulted in over 80% gene knockdown (FIG. 10). To test the therapeutic efficacy, D1D2-targeted LNPs or mD1D2-targeted LNPs loaded with siIFNγ (SEQ ID NO: 9-10) were injected into mice with colitis at days 4, 6, 8 and 10 upon initiation of colitis. A control mouse group injected with D1D2-LNPs loaded with a control siNC (SEQ ID NO: 18-19) was used to correct for possible therapeutic effects unrelated to IFNγ. In addition, mice without piroxicam were used as a healthy control and as positive treatment control in colitic mice a validated mAb against TNF-α was used (administered at days 4, 6, 8 and 10). The antibody against TNF-α was used as a positive control for amelioration of colitis. Blocking of TNF-α with mAbs has been well validated and anti-TNF-α mAbs are currently used in the clinic [e.g. infliximab (Ferreiro, R. & Barreiro-de Acosta, Infliximab: Pharmacology, Uses and Limitations 10, 39-74, Nova Science Publishers, Inc., 2012)]. At day 11, mice were sacrificed and colitis severity was assessed in all groups (FIGS. 6A-H).

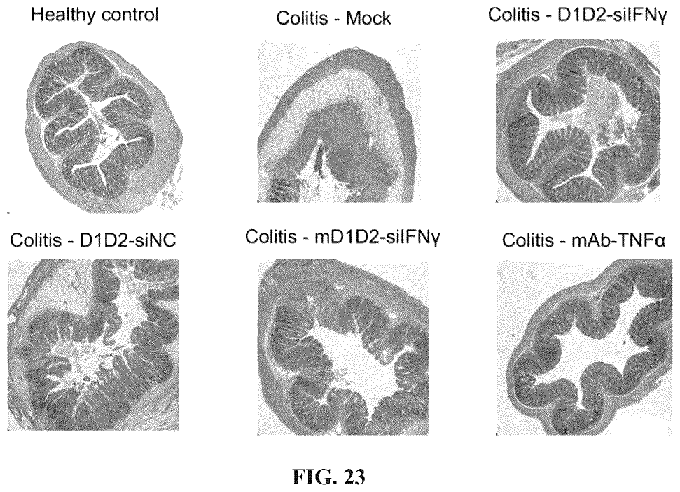

Starting from day 8, there was a significant difference in weight change in the D1D2-silFNγ group as compared to the negative control groups (FIG. 6B). Colonic levels of IFNγ decreased dramatically (˜2.5-fold) in D1D2-targeted LNPs loaded with siIFNγ as compared to the mD1D2-targeted LNPs control (FIG. 6D). Colonic IFNγ levels also decreased moderately in mice treated with anti-TNF-α, likely due to an overall decrease in intestinal inflammation. Because IFNγ affects TNF-α expression [Vila-del Sol, V., et al. J. Immunol. 181, 4461-4470 (2008)] and because these two cytokines have synergistic effects on NF-κB signaling [Wesemann, D. R. & Benveniste, E. N. J. Immunol. 171, 5313-5319 (2003)], an associated reduction of other pro-inflammatory cytokines is expected by silencing IFNγ. Mice treated with D1D2-targeted LNPs loaded with siIFNγ and mice treated with anti-TNF-α mAb showed a strong decrease in colonic TNF-α levels (FIG. 6C). The colonic TNF-α levels in mice treated with the mAb against TNF-α (positive control) was even a bit lower than the D1D2-targeted LNPs loaded with siIFNγ group (p<0.05). Furthermore, blood IL-6 and IL-1β levels decreased dramatically and equally both in mice treated with D1D2-targeted LNPs loaded with siIFNγ and mice treated with anti-TNF-α mAb (FIGS. 6E-F). Colon length (colon shortening is an important marker of colonic inflammation) was significantly increased (p<0.0001) in the D1D2-targeted LNPs loaded with siIFNγ group as compared to the mD1D2-targeted LNPs control (FIG. 6G). A significantly lower colon histological score (p<0.0001) further supported the improved therapeutic outcome in mice treated with D1D2-targeted LNPs loaded with siIFNγ (FIG. 6H). These results were also in accordance with visual examination of photomicrographs of sections of the colon (FIG. 23). Taken together, the data demonstrated a strong therapeutic response in mice treated with D1D2-targeted LNPs loaded with siIFNγ while the mutated control (mD1D2) did not lead to any significant improved therapeutic outcome.

### Example 4

### Generation of Lipid Nanoparticles Comprising a MAdCAM-1 Amino Acid Seuqence as a Targeting Moiety

In order to avoid use of rat IgG Fc regions which could render MadCAM-1-Fc fusion protein immunogenic in a human subject, alternative LNP conjugation strategies are evaluated. A non-limiting example include generating a MadCAM-1-Fc fusion protein using the Fc region of human IgG. Alternatively, a small non-immunogenic peptide is integrated into the MadCAM-1 recombinant protein and a humanized scFv against this tag is either chemically conjugated to the LNP or a lipidated humanized scFv against this tag is used (similarly to the ASSET described hereinabove).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Elinav, E. & Peer, D. Harnessing nanomedicine for mucosal theranostics-a silver bullet at last? ACS Nano 7, 2883-90 (2013).
2. Hanauer, S. B. et al. Incidence and importance of antibody responses to infliximab after maintenance or episodic treatment in Crohn's disease. Clin. Gastroenterol. Hepatol. 2, 542-553 (2004).
3. Baert, F. et al. Influence of Immunogenicity on the Long-Term Efficacy of Infliximab in Crohn's Disease. N. Engl. J. Med. 348, 601-608 (2003).
4. Kedmi, R. et al. A modular platform for targeted RNAi therapeutics. Nat. Nanotechnol. 13, 214-219 (2018).
5. Veiga, N. et al. Cell specific delivery of modified mRNA expressing therapeutic proteins to leukocytes. Nat. Commun. 9, (2018).
6. Whitehead, K. A., Langer, R. & Anderson, D. G. Knocking down barriers: advances in siRNA delivery. Nat. Rev. Drug Discov. 8, 129-138 (2009).
7. Fenske, D. B., Chonn, A. & Cullis, P. R. Liposomal Nanomedicines: An Emerging Field. Toxicol. Pathol. 36, 21-29 (2008).
8. Ledford, H. Gene silencing technology gets first drug approval after 20-year wait. Nature 560, 291-292 (2018).
9. Ramishetti, S. et al. Systemic Gene Silencing in Primary T Lymphocytes Using Targeted Lipid Nanoparticles. (2015).
10. Peer, D., Park, E. J., Morishita, Y., Carman, C. V & Shimaoka, M. Systemic leukocyte-directed siRNA delivery revealing cyclin D1 as an anti-inflammatory target. Science 319, 627-30 (2008).
11. Meenan, J. et al. Altered expression of alpha 4 beta 7, a gut homing integrin, by circulating and mucosal T cells in colonic mucosal inflammation. Gut 40, 241-246 (1997).
12. Yu, Y. et al. Structural specializations of α4β7, an integrin that mediates rolling adhesion. J. Cell Biol. 196, 131-46 (2012).
13. Sun, H. et al. Distinct chemokine signaling regulates integrin ligand specificity to dictate tissue-specific lymphocyte homing. Dev. Cell 30, 61-70 (2014).
14. Green, N. et al. Mutational analysis of MAdCAM-1/alpha4beta7 interactions reveals significant binding determinants in both the first and second immunuglobulin domains. Cell Adhes. Commun. 7, 167-81 (1999).
15. Shyjan, A. M., Bertagnolli, M., Kenney, C. J. & Briskin, M. J. Human mucosal addressin cell adhesion molecule-1 (MAdCAM-1) demonstrates structural and functional similarities to the alpha 4 beta 7-integrin binding domains of murine MAdCAM-1, but extreme divergence of mucin-like sequences. J. Immunol. 156, 2851-7 (1996).
16. Berlin, C. et al. α4β7 integrin mediates lymphocyte binding to the mucosal vascular addressin MAdCAM-1. Cell 74, 185-195 (1993).
17. Dearling, J. L. J. et al. Detection of intestinal inflammation by MicroPET imaging using a 64Cu-labeled antiβ7 integrin antibody. Inflamm. Bowel Dis. 16, 1458-1466 (2010).
18. Dearling, J. L. J., Daka, A., Veiga, N., Peer, D. & Packard, A. B. Colitis ImmunoPET: Defining Target Cell Populations and Optimizing Pharmacokinetics. Inflamm. Bowel Dis. 22, 529-538 (2016).
19. Belliveau, N. M. et al. Microfluidic Synthesis of Highly Potent Limit-size Lipid Nanoparticles for In Vivo Delivery of siRNA. Mol. Ther. Nucleic Acids 1, e37 (2012).

### 20. Cohen, Z. R., Ramishetti, S., Peshes-yaloz, N., Goldsmith, M. & Wohl, A. Localized RNAi Therapeutics of Chemoresistant Grade IV Glioma Using Hyaluronan-Grafted Lipid-Based. 1581-1591 (2015).

21. Veiga, N. et al. Leukocyte-specific siRNA delivery revealing IRF8 as a potential anti-inflammatory target. J. Control. Release 313, 33-41 (2019).

### 22. Chassaing, B., Aitken, J. D., Malleshappa, M. & Vijay-Kumar, M. Dextran Sulfate Sodium (DSS)-Induced Colitis in Mice. in Current Protocols in Immunology 104, 15.25.1-15.25.14 (John Wiley & Sons, Inc., 2014).

23. Berg, D. J. et al. Rapid development of colitis in NSAID-treated IL-10 deficient mice. Gastroenterology 123, 1527-1542 (2002).
24. Holgersen, K., Kvist, P. H., Markholst, H., Kornerup Hansen, A. & Holm, T. L. Characterisation of enterocolitis in the piroxicam-accelerated interleukin-10 knock out mouse—A model mimicking inflammatory bowel disease ⋆. 8, 147-160 (2012).
25. Ito, R. et al. Interferon-gamma is causatively involved in experimental inflammatory bowel disease in mice. Clin. Exp. Immunol. 146, 330-8 (2006).
26. Shimaoka, M. et al. AL-57, a ligand-mimetic antibody to integrin LFA-1, reveals chemokine-induced affinity up-regulation in lymphocytes. Proc. Natl. Acad. Sci. U. S. A. 103, 13991-13996 (2006).
27. Qi, J. et al. Identification, characterization, and epitope mapping of human monoclonal antibody J19 that specifically recognizes activated integrin α4β7. (2012). doi:10.1074/jbc.M112.341263
28. Kinashi, T. Intracellular signalling controlling integrin activation in lymphocytes. Nature Reviews Immunology 5, 546-559 (2005).
29. Eun, J. P. et al. Aberrant activation of integrin α4β7 suppresses lymphocyte migration to the gut. J. Clin. Invest. 117, 2526-2538 (2007).
30. Yang, Y. et al. Construction and Adhesive Properties of a Soluble MAdCAM-1-Fc Chimera Expressed in a Baculovirus System: Phylogenetic Conservation of Receptor-Ligand Interaction. 235-247 (1995).
31. Rungta, R. L. et al. Lipid Nanoparticle Delivery of siRNA to Silence Neuronal Gene

Expression in the Brain. Mol. Ther. Nucleic Acids 2, e136 (2013).

32. McCall, M. J., Diril, H. & Meares, C. F. Simplified Method for Conjugating Macrocyclic Bifunctional Chelating Agents to Antibodies via 2-Iminothiolane. Bioconjugate Chemistry 1, 222-226 (1990).
33. Loening, A. M. & Gambhir, S. S. AMIDE: A Free Software Tool for Multimodality Medical Image Analysis. Mol. Imaging 2, 131-137 (2003).

What is claimed is:

1. A polypeptide comprising an amino acid sequence of MAdCAM-1 capable of binding α4β7 integrin, wherein said polypeptide is in association with a therapeutic moiety, wherein said therapeutic moiety is not a constant region of an antibody.

2. A targeted particle comprising a therapeutic and/or a detectable moiety, said particle is attached to a polypeptide comprising an amino acid sequence of MAdCAM-1.

3. The polypeptide of claim 1, wherein said amino acid sequence of MAdCAM-1 does not comprise a functional Mucin-like domain.

4. The polypeptide of claim 1, wherein said amino acid sequence of MAdCAM-1 is more than 5 amino acids in length.

5. The polypeptide if claim 1, wherein said therapeutic moiety is attached to said polypeptide via a linker.

6. The polypeptide of claim 1, wherein said therapeutic moiety is attached to-, or encapsulated in a particle.

7. The polypeptide of claim 6, wherein said particle is a lipid particle.

8. The polypeptide of claim 1, wherein said therapeutic moiety is a polynucleotide, a small molecule or a polypeptide.

9. The polypeptide of claim 1, wherein said therapeutic moiety is an RNA silencing agent.

10. A method of treating a disease associated with inflammation mediated by MAdCAM-1 in a subject in need thereof, the method comprising administering to the subject a therapeutically effective amount of the polypeptide of claim 1, thereby treating the disease associated with inflammation mediated by MAdCAM-1.

11. The method of claim 10, wherein said disease is inflammatory bowel disease (IBD).

12. A polypeptide comprising an amino acid sequence of MAdCAM-1 capable of binding α4β7 integrin, wherein said polypeptide is in association with a detectable moiety, wherein said amino acid sequence of MAdCAM-1 is more than 5 amino acids long and does not comprise a functional Mucin-like domain.

13. The polypeptide of claim 12, wherein said detectable moiety is attached to said polypeptide via a linker.

14. The polypeptide of claim 1, wherein said MAdCAM-1 is human MAdCAM-1.

15. The polypeptide of claim 1, wherein said amino acid sequence of MAdCAM-1 comprises:
(i) an amino acid sequence of a first Ig-like domain (D1) located N to C in a full length MAdCAM-1;
(ii) an amino acid sequence of a second Ig-like domain (D2) located N to C in a full length MAdCAM-1; or
(iii) an amino acid sequence of a first Ig-like domain (D1) and a second Ig-like domain (D2) located N to C in a full length MAdCAM-1.

16. The polypeptide of claim 1, wherein said amino acid sequence of MAdCAM-1 comprises SEQ ID NO: 21.

17. The polypeptide of claim 1, wherein said amino acid sequence of MAdCAM-1 comprises SEQ ID NO: 22.

18. A method of diagnosing inflammation mediated by MAdCAM-1 in a subject in need, the method comprising:
(a) administering to the subject the polypeptide of claim 12; and
(b) determining an amount of said polypeptide in a suspected inflamed tissue of the subject following said administering,
wherein an amount above a predetermined threshold is indicative of presence of inflammation mediated by MAdCAM-1 in said tissue of said subject.

19. A method of monitoring efficacy of treatment against inflammation mediated by MAdCAM-1 in a subject in need, the method comprising:
(a) administering to the subject the polypeptide of claim 12 following said treatment; and
(b) determining an amount of said polypeptide or said particle in a suspected inflamed tissue of the subject following said administering,
wherein a decrease in the amount following said treatment beyond a predetermined threshold is indicative of reduction in inflammation and efficaciousness of the treatment.

20. The polypeptide of claim 1, wherein said polypeptide comprises an amino acid sequence of a constant region of an antibody.


SEQUENCE LISTING (41 sequences)

SEQ ID NO: 1 - DNA, 45 bp, synthetic construct
  misc_feature 1..45: note = Single strand DNA oligonucleotide
  source 1..45: mol_type = other DNA
gaaaacctgtactttcagggcgctgaaacaacagccccatctgtc

SEQ ID NO: 2 - DNA, 64 bp, synthetic construct
  misc_feature 1..64: note = Single strand DNA oligonucleotide
  source 1..64: mol_type = other DNA
cctctagagtcgactggtaccgatatcagatctatcgatgtcatttaccaggagagtgggagag

SEQ ID NO: 3 - DNA, 51 bp, synthetic construct
  misc_feature 1..51: note = Single strand DNA oligonucleotide
  source 1..51: mol_type = other DNA
ctggtcgctgtgctcaaaggtgtccagtgtgactacaaagacgatgacgac

SEQ ID NO: 4 - DNA, 50 bp, synthetic construct
  misc_feature 1..50: note = Single strand DNA oligonucleotide
  source 1..50: mol_type = other DNA
gatcgaacccttgtcgaggtcgggggatcctcatttaccaggagagtggg

SEQ ID NO: 5 - DNA, 40 bp, synthetic construct
  misc_feature 1..40: note = Single strand DNA oligonucleotide
  source 1..40: mol_type = other DNA
gtgcactggcgtggtctggccaccagcttgggcagtgtac

SEQ ID NO: 6 - DNA, 40 bp, synthetic construct
  misc_feature 1..40: note = Single strand DNA oligonucleotide
  source 1..40: mol_type = other DNA
gtacactgcccaagctggtggccagaccacgccagtgcac

SEQ ID NO: 7 - DNA, 60 bp, synthetic construct
  misc_feature 1..60: note = Single strand DNA oligonucleotide
  source 1..60: mol_type = other DNA
gaaaacctgtactttcagggcggttctggtgtgccaagggaatgcaatccttgtggatgt

SEQ ID NO: 8 - DNA, 48 bp, synthetic construct
  misc_feature 1..48: note = Single strand DNA oligonucleotide
  source 1..48: mol_type = other DNA
accagaaccacctagtactggaatctcctttctatgggtcagcaccag

SEQ ID NO: 9 - RNA, 25 nt, synthetic construct
  ncRNA 1..25: ncRNA_class = siRNA
  modified_base 1: mod_base = OTHER; note = 2'-O-METHYL RIBONUCLEOTIDE
  modified_base 2: mod_base = OTHER; note = 2'-O-METHYL RIBONUCLEOTIDE
  modified_base 4: mod_base = OTHER; note = 2'-O-METHYL RIBONUCLEOTIDE
  modified_base 6: mod_base = OTHER; note = 2'-O-METHYL RIBONUCLEOTIDE
  modified_base 12: mod_base = OTHER; note = 2'-O-METHYL RIBONUCLEOTIDE
  modified_base 14: mod_base = OTHER; note = 2'-O-METHYL RIBONUCLEOTIDE
  modified_base 16: mod_base = OTHER; note = 2'-O-METHYL RIBONUCLEOTIDE
  modified_base 18: mod_base = OTHER; note = 2'-O-METHYL RIBONUCLEOTIDE
  modified_base 24: mod_base = OTHER; note = 2'-O-METHYL RIBONUCLEOTIDE
  misc_feature 25: note = is a DEOXYRIBONUCLEOTIDE INSTEAD OF A RIBONUCLEOTIDE
  source 1..25: mol_type = other RNA
cattcatgagtattgccaagtttga

SEQ ID NO: 10 - RNA, 27 nt, synthetic construct
  misc_feature 1..27: note = IFNg siRNA Anrisense
  modified_base 3: mod_base = OTHER; note = 2'-O-METHYL RIBONUCLEOTIDE
  modified_base 9: mod_base = OTHER; note = 2'-O-METHYL RIBONUCLEOTIDE
  modified_base 11: mod_base = OTHER; note = 2'-O-METHYL RIBONUCLEOTIDE
  modified_base 13: mod_base = OTHER; note = 2'-O-METHYL RIBONUCLEOTIDE
  modified_base 23: mod_base = OTHER; note = 2'-O-METHYL RIBONUCLEOTIDE
  modified_base 25..27: mod_base = OTHER; note = 2'-O-METHYL RIBONUCLEOTIDE
  source 1..27: mol_type = other RNA
  ncRNA 1..27: ncRNA_class = siRNA
tcaaacttggcaatactcatgaatgca

SEQ ID NO: 11 - protein, 94 aa, synthetic construct
  REGION 1..94: note = Murine D1 domain
QSFQVNPPESEVAVAMGTSLQITCSMSCDEGVARVHWRGLDTSLGSVQTLPGSSILSVRGMLSDTGTPVCVGSCGSRSFQHSVKILVYAFPDQL

SEQ ID NO: 12 - protein, 105 aa, synthetic construct
  REGION 1..105: note = Murine D2 domain
VVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPVL

SEQ ID NO: 13 - protein, 225 aa, synthetic construct
  REGION 1..225: note = Fc domain of rat IgG2a (including the hinge, excluding CH1)
VPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 14 - DNA, 1440 bp, synthetic construct
  misc_feature 1..1440: note = NA sequence of open reading frame of the D1D2-Fc construct.
  source 1..1440: mol_type = other DNA
atggagactgggctgcgctggcttctcctggtcgctgtgctcaaaggtgtccagtgtgactacaaagacgatgacgacaagcttgcggccgcgcagtccttccaggtgaacccccctgagtctgaggtagctgtggccatgggcacatccctccagatcacctgcagcatgtcctgtgacgagggtgtagcccgggtgcactggcgtggtctggacaccagcttgggcagtgtacagaccctcccaggcagcagtatcctctctgtacggggcatgctgtcagacacaggcactcctgtgtgtgtgggctcctgcgggagtcgaagcttccagcactccgtgaagatccttgtgtatgccttcccagaccagctggtggtgtccccggagttccttgtacctggacaggaccaggtggtgtcctgcacggcccacaacatctggcctgcagacccgaacagtctctcctttgccctgctactgggagagcagagactggagggtgcccaagccctggaaccagagcaagaagaggagatacaagaggctgagggcacaccactgttccgaatgacacaacgctggcggttaccctccctggggacccctgcccctcctgcccttcactgccaggtcaccatgcagctgcccaaactggtgctgacccatagaaaggagattccagtgctgggcggaggcgggagcggaggcggagggtccggaggcggcgggagcgtgccaagggaatgcaatccttgtggatgtacaggctcagaagtgtcatctgtcttcatcttccccccaaagaccaaagatgtgctcaccatcactctgactcctaaggtcacgtgtgttgtggtagacattagccagaatgatcccgaggtccggttcagctggtttatagatgacgtggaagtccacacagctcagactcatgccccggagaagcagtccaacagcactttacgctcagtcagtgaactccccatcgtgcaccgggactggctcaatggcaagacgttcaaatgcaaagtcaacagtggagcattccctgcccccatcgagaaaagcatctccaaacccgaaggcacaccacgaggtccacaggtatacaccatggcgcctcccaaggaagagatgacccagagtcaagtcagtatcacctgcatggtaaaaggcttctatcccccagacatttatacggagtggaagatgaacgggcagccacaggaaaactacaagaacactccacctacgatggacacagatgggagttacttcctctacagcaagctcaatgtaaagaaagaaacatggcagcagggaaacactttcacgtgttctgtgctgcatgagggcctgcacaaccaccatactgagaagagtctctcccactctcctggtaaaggctccggccatcatcaccaccaccattga

SEQ ID NO: 15 - protein, 479 aa, synthetic construct
  REGION 1..479: note = Protein sequence of the D1D2-Fc construct.
METGLRWLLLVAVLKGVQCDYKDDDDKLAAAQSFQVNPPESEVAVAMGTSLQITCSMSCDEGVARVHWRGLDTSLGSVQTLPGSSILSVRGMLSDTGTPVCVGSCGSRSFQHSVKILVYAFPDQLVVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPVLGGGGSGGGGSGGGGSVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGKGSGHHHHHH

SEQ ID NO: 16 - DNA, 1440 bp, synthetic construct
  misc_feature 1..1440: note = NA sequence of open reading frame of the mD1D2-Fc construct.
  source 1..1440: mol_type = other DNA
atggagactgggctgcgctggcttctcctggtcgctgtgctcaaaggtgtccagtgtgactacaaagacgatgacgacaagcttgcggccgcgcagtccttccaggtgaacccccctgagtctgaggtagctgtggccatgggcacatccctccagatcacctgcagcatgtcctgtgacgagggtgtagcccgggtgcactggcgtggtctggccaccagcttgggcagtgtacagaccctcccaggcagcagtatcctctctgtacggggcatgctgtcagacacaggcactcctgtgtgtgtgggctcctgcgggagtcgaagcttccagcactccgtgaagatccttgtgtatgccttcccagaccagctggtggtgtccccggagttccttgtacctggacaggaccaggtggtgtcctgcacggcccacaacatctggcctgcagacccgaacagtctctcctttgccctgctactgggagagcagagactggagggtgcccaagccctggaaccagagcaagaagaggagatacaagaggctgagggcacaccactgttccgaatgacacaacgctggcggttaccctccctggggacccctgcccctcctgcccttcactgccaggtcaccatgcagctgcccaaactggtgctgacccatagaaaggagattccagtgctgggcggaggcgggagcggaggcggagggtccggaggcggcgggagcgtgccaagggaatgcaatccttgtggatgtacaggctcagaagtgtcatctgtcttcatcttccccccaaagaccaaagatgtgctcaccatcactctgactcctaaggtcacgtgtgttgtggtagacattagccagaatgatcccgaggtccggttcagctggtttatagatgacgtggaagtccacacagctcagactcatgccccggagaagcagtccaacagcactttacgctcagtcagtgaactccccatcgtgcaccgggactggctcaatggcaagacgttcaaatgcaaagtcaacagtggagcattccctgcccccatcgagaaaagcatctccaaacccgaaggcacaccacgaggtccacaggtatacaccatggcgcctcccaaggaagagatgacccagagtcaagtcagtatcacctgcatggtaaaaggcttctatcccccagacatttatacggagtggaagatgaacgggcagccacaggaaaactacaagaacactccacctacgatggacacagatgggagttacttcctctacagcaagctcaatgtaaagaaagaaacatggcagcagggaaacactttcacgtgttctgtgctgcatgagggcctgcacaaccaccatactgagaagagtctctcccactctcctggtaaaggctccggccatcatcaccaccaccattga

SEQ ID NO: 17 - protein, 479 aa, synthetic construct
  REGION 1..479: note = Protein sequence of the mD1D2-Fc construct.
METGLRWLLLVAVLKGVQCDYKDDDDKLAAAQSFQVNPPESEVAVAMGTSLQITCSMSCDEGVARVHWRGLATSLGSVQTLPGSSILSVRGMLSDTGTPVCVGSCGSRSFQHSVKILVYAFPDQLVVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPVLGGGGSGGGGSGGGGSVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGKGSGHHHHHH

SEQ ID NO: 18 - RNA, 25 nt, synthetic construct
  misc_feature 1..25: note = Negative Control siRNA sense
  modified_base 1..2: mod_base = OTHER; note = 2'-O-METHYL RIBONUCLEOTIDE
  modified_base 4: mod_base = OTHER; note = 2'-O-METHYL RIBONUCLEOTIDE
  modified_base 6: mod_base = OTHER; note = 2'-O-METHYL RIBONUCLEOTIDE
  modified_base 12: mod_base = OTHER; note = 2'-O-METHYL RIBONUCLEOTIDE
  modified_base 14: mod_base = OTHER; note = 2'-O-METHYL RIBONUCLEOTIDE
  modified_base 16: mod_base = OTHER; note = 2'-O-METHYL RIBONUCLEOTIDE
  modified_base 18: mod_base = OTHER; note = 2'-O-METHYL RIBONUCLEOTIDE
  modified_base 24: mod_base = OTHER; note = 2'-O-METHYL RIBONUCLEOTIDE
  misc_feature 25: note = IS A DEOXYRIBONUCLEOTIDE INSTEAD OF A RIBONUCLEOTIDE
  source 1..25: mol_type = other RNA
  ncRNA 1..25: ncRNA_class = siRNA
catattgcgcgtatagtcgcgttag

SEQ ID NO: 19 - RNA, 27 nt, synthetic construct
  misc_feature 1..27: note = Negative Control siRNA antisense
  modified_base 3: mod_base = OTHER; note = 2'-O-METHYL RIBONUCLEOTIDE
  modified_base 9: mod_base = OTHER; note = 2'-O-METHYL RIBONUCLEOTIDE
  modified_base 11: mod_base = OTHER; note = 2'-O-METHYL RIBONUCLEOTIDE
  modified_base 13: mod_base = OTHER; note = 2'-O-METHYL RIBONUCLEOTIDE
  modified_base 23: mod_base = OTHER; note = 2'-O-METHYL RIBONUCLEOTIDE
  modified_base 25..27: mod_base = OTHER; note = 2'-O-METHYL RIBONUCLEOTIDE
  source 1..27: mol_type = other RNA
  ncRNA 1..27: ncRNA_class = siRNA
ctaacgcgactatacgcgcaatatggt

SEQ ID NO: 20 - RNA, 21 nt, synthetic construct
  misc_feature 1..21: note = siRNA CD45 Sense
  modified_base 1..2: mod_base = OTHER; note = 2'-O-METHYL RIBONUCLEOTIDE
  modified_base 5..6: mod_base = OTHER; note = 2'-O-METHYL RIBONUCLEOTIDE
  modified_base 10..13: mod_base = OTHER; note = 2'-O-METHYL RIBONUCLEOTIDE
  modified_base 18: mod_base = OTHER; note = 2'-O-METHYL RIBONUCLEOTIDE
  misc_feature 20..21: note = ARE DEOXYRIBONUCLEOTIDES
  modified_base 20^21: mod_base = OTHER; note = linked via a phosphorthioate bond
  source 1..21: mol_type = other RNA
  ncRNA 1..21: ncRNA_class = siRNA
ctggctgaatttcagagcatt

SEQ ID NO: 21 - protein, 4 aa, synthetic construct
  REGION 1..4: note = 4 aa MAdCAM-1 D1 binding sequence
LDTS

SEQ ID NO: 22 - protein, 199 aa, synthetic construct
  REGION 1..199: note = MAdCAM01 D1D2 sequence
QSFQVNPPESEVAVAMGTSLQITCSMSCDEGVARVHWRGLDTSLGSVQTLPGSSILSVRGMLSDTGTPVCVGSCGSRSFQHSVKILVYAFPDQLVVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPVL

SEQ ID NO: 23 - protein, 405 aa, Mus musculus
MESILSLLLSLSLVPYQLSRGQSFQVNPPESEVAVAMGTSLQITCSMSCDEGVARVHWRGRDTSLGSVQTLPGSSILSVRGMLSDTGTPVCVGSCGSRSFQHSVKILVYAFPDQLVVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPVLQSQTSPKPPNTTSAEPYILTSSSTAEAVSTGLNITTLPSAPPYPKLSPRTLSSEGPCRPKIHQDLEAGWELLCEASCGPGVTVRWTLAPGDLATYHKREAGAQAWLSVLPPGPMVEGWFQCRQDPGGEVTNLYVPGQVTPNSSSTVVLWIGSLVLGLLALVFLAYRLWKCYRPGPRPDTSSCTHL

SEQ ID NO: 24 - protein, 261 aa, Mus musculus
MESILALLLALALVPYQLSRGQSFQVNPPESEVAVAMGTSLQITCSMSCDEGVARVHWRGLDTSLGSVQTLPGSSILSVRGMLSDTGTPVCVGSCGSRSFQHSVKILVYAFPDQLVVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPASTVVLWIGSLVLGLLALVFLAYRLWKCYRPGPRPDTSSCTHL

SEQ ID NO: 25 - protein, 405 aa, Mus musculus
MESILALLLALALVPYQLSRGQSFQVNPPESEVAVAMGTSLQITCSMSCDEGVARVHWRGLDTSLGSVQTLPGSSILSVRGMLSDTGTPVCVGSCGSRSFQHSVKILVYAFPDQLVVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPVLQSQTSPKPPNTTSAEPYILTSSSTAEAVSTGLNITTLPSAPPYPKLSPRTLSSEGPCRPKIHQDLEAGWELLCEASCGPGVTVRWTLAPGDLATYHKREAGAQAWLSVLPPGPMVEGWFQCRQDPGGQVTNLYVPGQVTPNSSSTVVLWIGSLVLGLLALVFLAYRLWKCYRPGPRPDTSSCTHL

SEQ ID NO: 26 - protein, 382 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 27 - protein, 295 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPASKPAGDQLPAALWTSSAVLGLLLLALPTYHLWKRCRHLAEDDTHPPASLRLLPQVSAWAGLRGTGQVGISPS

SEQ ID NO: 28 - protein, 344 aa, synthetic construct
  REGION 1..344: note = Extracellular domain of Mouse MAdCAM-1
QSFQVNPPESEVAVAMGTSLQITCSMSCDEGVARVHWRGLDTSLGSVQTLPGSSILSVRGMLSDTGTPVCVGSCGSRSFQHSVKILVYAFPDQLVVSPEFLVPGQDQVVSCTAHNIWPADPNSLSFALLLGEQRLEGAQALEPEQEEEIQEAEGTPLFRMTQRWRLPSLGTPAPPALHCQVTMQLPKLVLTHRKEIPVLQSQTSPKPPNTTSAEPYILTSSSTAEAVSTGLNITTLPSAPPYPKLSPRTLSSEGPCRPKIHQDLEAGWELLCEASCGPGVTVRWTLAPGDLATYHKREAGAQAWLSVLPPGPMVEGWFQCRQDPGGQVTNLYVPGQVTPNSSST

SEQ ID NO: 29 - protein, 321 aa, synthetic construct
  REGION 1..321: note = Extracellular domain of HUman MAdCAM-1
VKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPEPPNTTSPESPDTTSPESPDTTSQEPPDTTSQEPPDTTSQEPPDTTSPEPPDKTSPEPAPQQGSTHTPRSPGSTRTRRPEISQAGPTQGEVIPTGSSKPAGDQLP

SEQ ID NO: 30 - protein, 20 aa, synthetic construct
  REGION 1..20: note = Transmembrane domain of Mouse MAdCAM-1
VVLWIGSLVLGLLALVFLAY

SEQ ID NO: 31 - protein, 20 aa, synthetic construct
  REGION 1..20: note = Transmembrane domain of Human MAdCAM-1
AALWTSSAVLGLLLLALPTY

SEQ ID NO: 32 - protein, 20 aa, synthetic construct
  REGION 1..20: note = Intracellular domain of Mouse MAdCAM-1
RLWKCYRPGPRPDTSSCTHL

SEQ ID NO: 33 - protein, 43 aa, synthetic construct
  REGION 1..43: note = Intracellular domain of Human MAdCAM-1
HLWKRCRHLAEDDTHPPASLRLLPQVSAWAGLRGTGQVGISPS

SEQ ID NO: 34 - protein, 94 aa, synthetic construct; identical to SEQ ID NO: 11
  REGION 1..94: note = D1 domain of mouse MAdCAM-1

SEQ ID NO: 35 - protein, 90 aa, synthetic construct
  REGION 1..90: note = D1 domain of HUMAN MAdCAM-1
VKPLQVEPPEPVVAVALGASRQLTCRLACADRGASVQWRGLDTSLGAVQSDTGRSVLTVRNASLSAAGTRVCVGSCGGRTFQHTVQLLVY

SEQ ID NO: 36 - protein, 105 aa, synthetic construct; identical to SEQ ID NO: 12
  REGION 1..105: note = D2 domain of mouse MAdCAM-1

SEQ ID NO: 37 - protein, 113 aa, synthetic construct
  REGION 1..113: note = D2 domain of HUMAN MAdCAM-1
AFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLH

SEQ ID NO: 38 - protein, 108 aa, synthetic construct
  REGION 1..108: note = Mouse D3 domain
PSAPPYPKLSPRTLSSEGPCRPKIHQDLEAGWELLCEASCGPGVTVRWTLAPGDLATYHKREAGAQAWLSVLPPGPMVEGWFQCRQDPGGQVTNLYVPGQVTPNSSST

SEQ ID NO: 39 - protein, 37 aa, synthetic construct
  REGION 1..37: note = Mucin-like domain of mouse MAdCAM-1
QSQTSPKPPNTTSAEPYILTSSSTAEAVSTGLNITTL

SEQ ID NO: 40 - protein, 118 aa, synthetic construct
  REGION 1..118: note = Mucin-like domain of HUMAN MAdCAM-1
SPTSPEPPDTTSPEPPNTTSPESPDTTSPESPDTTSQEPPDTTSQEPPDTTSQEPPDTTSPEPPDKTSPEPAPQQGSTHTPRSPGSTRTRRPEISQAGPTQGEVIPTGSSKPAGDQLP

SEQ ID NO: 41 - RNA, 21 nt, synthetic construct
  misc_feature 1..21: note = siRNA CD45 antisense
  modified_base 13: mod_base = OTHER; note = 2'-O-METHYL RIBONUCLEOTIDE
  modified_base 17: mod_base = OTHER; note = 2'-O-METHYL RIBONUCLEOTIDE
  misc_feature 20..21: note = ARE DEOXYRIBONUCLEOTIDES INSTEAD OF A RIBONUCLEOTIDES
  modified_base 20^21: mod_base = OTHER; note = linked via a phosphorthioate bond
  source 1..21: mol_type = other RNA
  ncRNA 1..21: ncRNA_class = siRNA
tgctctgaaattcagccagtt